(19) United States
(12) Patent Application Publication
(10) Pub. No.: US 2014/0073651 A1
(43) Pub. Date: Mar. 13, 2014

(54) BENZAMIDE DERIVATIVES AS P2X7 RECEPTOR ANTAGONISTS

(71) Applicants: Kurt Hilpert, Allschwil (CH); Francis Hubler, Allschwil (CH); Mark Murphy, Allschwil (CH); Dorte Renneberg, Allschwil (CH)

(72) Inventors: Kurt Hilpert, Allschwil (CH); Francis Hubler, Allschwil (CH); Mark Murphy, Allschwil (CH); Dorte Renneberg, Allschwil (CH)

(73) Assignee: Actelion Pharmaceuticals Ltd., Allschwil (CH)

(21) Appl. No.: 14/001,052

(22) PCT Filed: Feb. 21, 2012
(86) PCT No.: PCT/IB2012/050780

§ 371 Date: Aug. 22, 2013

(30) Foreign Application Priority Data
Feb. 22, 2011 (IB) PCT/IB2011/050728

(51) Int. Cl. C07D 241/20; C07D 239/47; C07C 233/74; C07D 239/48; C07D 239/42; C07D 237/22; C07D 239/36

(52) U.S. Cl. CPC C07D 241/20; C07D 237/22; C07D 239/47; C07D 239/36; C07D 239/48; C07D 239/42; C07C 233/74
USPC 514/255.06; 514/247; 514/256; 514/269; 514/274; 514/275; 514/619; 544/239; 544/317; 544/319; 544/325; 544/329; 544/336; 544/408; 564/168

(57) ABSTRACT

The invention relates to benzamide derivatives of formula (I),

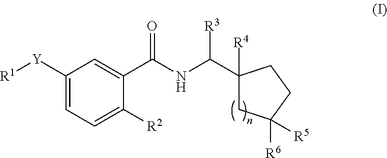

wherein R¹, R², R³, R⁴, R⁵, R⁶, n and Y are as defined in the description, their preparation and their use as pharmaceutically active compounds.

### CROSS REFERENCE TO RELATED APPLICATIONS

This application is a United States application under 35 U.S.C. 371 claiming benefit of PCT Application No. PCT/IB2012/050780, filed on Feb. 21, 2012, which claims the benefit of PCT Application No. PCT/IB2011/050728, filed on Feb. 22, 2011, the contents of each of which are incorporated herein by reference.

The present invention relates to benzamide derivatives of formula (I) and their use as pharmaceuticals. The invention also concerns related aspects including processes for the preparation of the compounds, pharmaceutical compositions containing one or more compounds of formula (I), and especially their use as P2X₇ receptor antagonists.

The P2X7 receptors (P2RX7) belong to the family of P2X ionotropic receptors that are activated by extracellular nucleotides, in particular adenosine triphosphate (ATP). P2RX7 is distinguished from other P2X family members by the high concentrations (mM range) of ATP required to activate it and its ability to form a large pore upon prolonged or repeated stimulation (North, R. A., Physiol. Rev. 2002, 82(4), 1013-67; Surprenant, A., Rassendren, F. et al., Science 1996, 272(5262), 735-8; Virginio, C., MacKenzie, A. et al., J. Physiol., 1999, 519, 335-46). P2RX7 is present on many cell types, especially ones known to be involved in inflammatory and immune processes. This is reflected within both the periphery and the CNS as Lipopolysaccharide S (LPS) priming of monocytes and microglia followed by ATP stimulation has been shown to lead to the local release and processing of IL1β and other family members including IL18 through a P2RX7 mediated mechanism. Indeed mice lacking the P2X7 receptor are unable to release IL1β following LPS priming and ATP stimulation providing further evidence of its role in this pathway (Solle, M., Labasi, J. et al., J. Biol. Chem., 2001, 276(1), 125-32). In addition L-selectin shedding from monocytes, macrophages and lymphocytes, degranulation in mast cells and apoptosis in lymphocytes are all associated with P2RX7 stimulation. P2RX7 is also expressed on epithelial and endothelial cells (Ferrari, D., Chiozzi, P. et al., Neuropharmacology 1997, 36(9), 1295-301; Wiley, J. S., Chen, J. R. et al., Ciba Found Symp. 1996, 198, 149-60 and 160-5; North, R. A., Physiol. Rev. 2002, 82(4), 1013-67). In addition to its role in the periphery it may have an important function in neurotransmission within the CNS through its activation on postsynaptic and/or presynaptic central and peripheral neurons and glia (Deuchars, S. A., Atkinson, L. et al., J. Neurosci. 2001, 21(18), 7143-52; Sperlagh, B., Kofalvi, A. et al., J. Neurochem. 2002, 81(6), 1196-211). Recent data that has emerged using in situ hybridization demonstrated that P2X7 receptor mRNA was widely distributed throughout the rat brain. Specifically, among the areas of high P2×7mRNA expression noted were the piriform cortex, hippocampus, pontine nuclei and the anterior horn of the spinal cord (Yu, Y., Ugawa, S. et al., Brain. Res. 2008, 1194, 45-55). Hence there is therapeutic rationale for the use of P2X7 ion channel blockers in the treatment of a variety of disease states. These include but are not limited to diseases associated with the central nervous system such as stroke or injury and diseases associated with neuro-degeneration and neuroinflammation such as Alzheimer's disease, Huntington's disease, epilepsy, Amyotrophic lateral sclerosis, acute spinal cord injury additionally to meningitis, sleep disorders, mood and anxiety disorders as well as chronic and neuropathic and inflammatory pain. Furthermore, peripheral inflammatory disorders and autoimmune diseases including but not limited to rheumatoid arthritis, osteoarthritis, psoriasis, allergic dermatitis, asthma, chronic obstructive pulmonary disease, airways hyper-responsiveness, septic shock, bronchitis, glomerulonephritis, irritable bowel disease, skin injury, lung emphysema, Limb girdle dystrophy type 2B, fibrosis, Syndrome of synovitis Acne Pustulosis, atherosclerosis, burn injury, spinal cord injury, Hyperostosis Osteitis, Crohn's disease, ulcerative colitis, growth and metastases of malignant cells, myoblastic leukaemia, diabetes, trauma, meningitis, osteoporosis, burn injury, ischemic heart disease, and varicose veins and trauma, are all examples where the involvement of P2X7 channels has been implicated. In addition a recent report suggests a link between P2RX7 and chronic, inflammatory and neuropathic pain (Chessell, I. P., Hatcher, J. P. et al., Pain, 2005, 114(3), 386-96). Overall, these findings indicate a role for the P2X7 receptor in the process of neuronal synaptic transmission and therefore a potential role for P2X7 antagonists as novel therapeutic tools to treat neuropathic pain.

In view of the above observations, there is significant requirement for P2X7 antagonists that can be efficiently used in treating neuropathic pain, chronic inflammatory pain, inflammation, and neurodegenerative conditions.

Different benzamide derivatives, which are also P2X₇ receptor antagonists, have been disclosed in WO 2003/042191, WO 2004/058270, WO 2004/058731, WO 2004/099146 and in WO 2005/019182.

Various embodiments of the invention are presented hereafter:

1) The present invention relates to benzamide derivatives of formula (I),

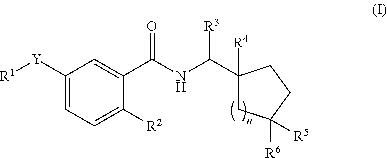

wherein
n represents 1, 2, 3 or 4 (and preferably 2, 3 or 4);
Y represents —C(R⁷R⁸)—, —N(R⁹)—, —O—, —S—, —S(O)—, or —S(O)₂—;
R¹ represents
a 5-membered heteroaryl group which is unsubstituted or mono- or di-substituted with (C₁-C₄)alkyl;
a 6-membered heteroaryl group which is unsubstituted or mono- or di-substituted, wherein the substituents are independently selected from the group consisting of halogen, hydroxy, (C₁-C₄)alkyl, (C₁-C₄)alkoxy, (C₁-C₄)alkylthio, (C₁-C₄)alkyl-sulfonyl, (C₁-C₄)alkyl-amino and di-[(C₁-C₄)alkyl]-amino;
a phenyl group which is unsubstituted or mono- or di-substituted with halogen; or
a heterocyclyl group which is unsubstituted or mono- or di-substituted with (C₁-C₄)alkyl or (C₁-C₂)alkoxy-(C₁-C₄)alkyl;
   R² represents chloro or methyl (and preferably chloro);
   R³ represents hydrogen and R⁴ represents hydroxy, hydroxy-(C₁-C₄)alkyl, —CONH₂ or (C₁-C₄)alkoxy (and preferably hydroxy, hydroxymethyl or methoxy); or
   R³ represents (C₁-C₄)alkyl or hydroxy-(C₁-C₄)alkyl (and preferably methyl or hydroxymethyl) and R⁴ represents hydrogen;
   R⁵ represents hydrogen or fluoro;
   R⁶ represents hydrogen or fluoro;
   R⁷ and R⁸ represent independently from each other hydrogen, fluoro, hydroxy or (C₁-C₄)alkyl, with the proviso that R⁸ is different from fluoro or hydroxy if R⁷ represents hydroxy;
   or R⁷ and R⁸ together represent an oxo-group; and
   R⁹ represents hydrogen, (C₁-C₄)alkyl, (C₁-C₂)alkoxy-(C₁-C₄)alkyl, (C₃-C₆)cycloalkyl-(C₁-C₄)alkyl, phenyl-(C₁-C₄)alkyl, or phenyloxy-(C₁-C₄)alkyl;
   and to the salts (in particular pharmaceutically acceptable salts) of such compounds.

The compounds of formula (I) according to embodiment 1) may contain one or more stereogenic or asymmetric centers, such as one or more asymmetric carbon atoms. Substituents at a double bond may be present in the (Z)- or (E)-configuration unless indicated otherwise. The compounds of formula (I) may thus be present as mixtures of stereoisomers or preferably as pure stereoisomers. Mixtures of stereoisomers may be separated in a manner known to a person skilled in the art.

The following paragraphs provide definitions of the various chemical moieties for the compounds according to the invention and are intended to apply uniformly throughout the specification and claims unless an otherwise expressly set out definition provides a broader or narrower definition.

The term “alkyl”, used alone or in combination, refers to a straight or branched chain alkyl group containing one to four carbon atoms. The term “(Cₓ—C_{y})alkyl” (x and y each being an integer), refers to an alkyl group as defined before containing x to y carbon atoms. For example a (C₁-C₄)alkyl group contains from one to four carbon atoms. Representative examples of alkyl groups include methyl, ethyl, n-propyl, iso-propyl, n-butyl, iso-butyl, sec-butyl and tert-butyl.

In case a (C₁-C₄)alkyl group is a substituent to a 5-membered heteroaryl group, the term “(C₁-C₄)alkyl” means (C₁-C₄)alkyl groups as defined above. Examples of said groups are methyl, ethyl, n-propyl, iso-propyl, n-butyl, iso-butyl, sec-butyl and tert-butyl. Preferred is methyl.

In case a (C₁-C₄)alkyl group is a substituent to a 6-membered heteroaryl group, the term “(C₁-C₄)alkyl” means (C₁-C₄)alkyl groups as defined above. Examples of said groups are methyl, ethyl, n-propyl, iso-propyl, n-butyl, iso-butyl, sec-butyl and tert-butyl. Preferred is methyl.

In case a (C₁-C₄)alkyl group is a substituent to a heterocyclyl group, the term “(C₁-C₄)alkyl” means (C₁-C₄)alkyl groups as defined above. Examples of said groups are methyl, ethyl, n-propyl, iso-propyl, n-butyl, iso-butyl, sec-butyl and tert-butyl. Preferred is methyl.

In case “R³” represents “(C₁-C₄)alkyl” the term means (C₁-C₄)alkyl groups as defined above. Examples of said groups are methyl, ethyl, n-propyl, iso-propyl, n-butyl, iso-butyl, sec-butyl and tert-butyl. Preferred is methyl.

In case “R⁷” or “R⁸” represent “(C₁-C₄)alkyl” the term means (C₁-C₄)alkyl groups as defined above. Examples of said groups are methyl, ethyl, n-propyl, iso-propyl, n-butyl, iso-butyl, sec-butyl and tert-butyl. Preferred is methyl.

In case “R⁹” represents “(C₁-C₄)alkyl” the term means (C₁-C₄)alkyl groups as defined above. Examples of said groups are methyl, ethyl, n-propyl, iso-propyl, n-butyl, iso-butyl, sec-butyl and tert-butyl. Preferred are methyl, ethyl, n-propyl and iso-butyl. More preferred are methyl and ethyl and most preferred is methyl.

The term “alkoxy”, used alone or in combination, refers to an alkyl-O— group wherein the alkyl group is as defined above. The term “(Cₓ—C_{y})alkoxy” (x and y each being an integer) refers to an alkoxy group as defined before containing x to y carbon atoms. For example a (C₁-C₄)alkoxy group contains from one to four carbon atoms. Representative examples of alkoxy groups include methoxy, ethoxy, n-propoxy, iso-propoxy, n-butoxy, iso-butoxy, sec-butoxy and tert-butoxy.

In case a (C₁-C₄)alkoxy group is a substituent to a 6-membered heteroaryl group, the term “(C₁-C₄)alkoxy” means (C₁-C₄)alkoxy groups as defined above. Examples of said groups are methoxy, ethoxy, n-propoxy, iso-propoxy, n-butoxy, iso-butoxy, sec-butoxy and tert-butoxy. Preferred is methoxy.

In case “R⁴” represents “(C₁-C₄)alkoxy” the term means (C₁-C₄)alkoxy groups as defined above. Examples of said groups are methoxy, ethoxy, n-propoxy, iso-propoxy, n-butoxy, iso-butoxy, sec-butoxy and tert-butoxy. Preferred is methoxy.

The term “alkylthio”, used alone or in combination, refers to an alkyl-S— group wherein the alkyl group is as defined above. The term “(Cₓ-C_{y})alkylthio” (x and y each being an integer) refers to an alkylthio group as defined before containing x to y carbon atoms. For example a (C₁-C₄)alkylthio group contains from one to four carbon atoms. Representative examples of alkylthio groups include methylthio, ethylthio, n-propylthio, iso-propylthio, n-butylthio, iso-butylthio, sec-butylthio and tert-butylthio.

In case a (C₁-C₄)alkylthio group is a substituent to a 6-membered heteroaryl group, the term “(C₁-C₄)alkylthio” means (C₁-C₄)alkylthio groups as defined above. Examples of said groups are methylthio, ethylthio, n-propylthio, iso-propylthio, n-butylthio, iso-butylthio, sec-butylthio and tert-butylthio. Preferred is methylthio.

The term “(C₁-C₄)alkyl-amino”, used alone or in combination, refers to an amino group (—NH₂) in which one hydrogen atom has been replaced by a (C₁-C₄)alkyl group as defined above. Representative examples of (C₁-C₄)alkyl-amino groups include methylamino, ethylamino, n-propylamino, iso-propylamino, n-butylamino, iso-butylamino, sec-butylamino and tert-butylamino. Preferred is methylamino.

The term “di-[(C₁-C₄)alkyl]-amino”, used alone or in combination, refers to an amino group (—NH₂) in which each of the two hydrogen atoms has been replaced by a (C₁-C₄)alkyl group as defined above, wherein the two (C₁-C₄)alkyl groups may be the same or different. Representative examples of di-[(C₁-C₄)alkyl]-amino groups include, but are not limited to, dimethylamino, methyl-ethyl-amino and diethylamino. Preferred is dimethylamino.

The term “(C₁-C₄)alkyl-sulfonyl”, used alone or in combination, refers to an (C₁-C₄)alkyl-S(O)₂— group wherein the (C₁-C₄)alkyl group is as defined above. Representative examples of (C₁-C₄)alkyl-sulfonyl groups include methyl-sulfonyl, ethyl-sulfonyl, n-propyl-sulfonyl, iso-propyl-sulfonyl, n-butyl-sulfonyl, iso-butyl-sulfonyl, sec-butyl-sulfonyl and tert-butyl-sulfonyl. Preferred is methyl-sulfonyl.

The term “hydroxy-(C₁-C₄)alkyl” refers to an alkyl group as defined before containing from one to four carbon atoms in which one hydrogen atom has been replaced with hydroxy. Examples of hydroxy-(C₁-C₄)alkyl groups include, but are not limited to, hydroxy-methyl, 1-hydroxy-ethyl, 2-hydroxy-ethyl, 1-hydroxy-propyl, 2-hydroxy-propyl, 3-hydroxy-propyl, 1-hydroxy-1-methyl-ethyl and 2-hydroxy-1-methyl-ethyl.

In case “R³” represents “hydroxy-(C₁-C₄)alkyl” the term means hydroxy-(C₁-C₄)alkyl groups as defined above. Examples of said groups include, but are not limited to, hydroxy-methyl, 1-hydroxy-ethyl, 2-hydroxy-ethyl, 1-hydroxy-propyl, 2-hydroxy-propyl, 3-hydroxy-propyl, 1-hydroxy-1-methyl-ethyl and 2-hydroxy-1-methyl-ethyl. Preferred is hydroxy-methyl.

In case “R⁴” represents “hydroxy-(C₁-C₄)alkyl” the term means hydroxy-(C₁-C₄)alkyl groups as defined above. Examples of said groups include, but are not limited to, hydroxy-methyl, 1-hydroxy-ethyl, 2-hydroxy-ethyl, 1-hydroxy-propyl, 2-hydroxy-propyl, 3-hydroxy-propyl, 1-hydroxy-1-methyl-ethyl and 2-hydroxy-1-methyl-ethyl. Preferred is hydroxy-methyl.

The term “(C₁-C₂)alkoxy-(C₁-C₄)alkyl” refers to an alkyl group as defined before containing from one to four carbon atoms in which one hydrogen atom has been replaced with (C₁-C₂)alkoxy as defined before. Examples of (C₁-C₂)alkoxy-(C₁-C₄)alkyl groups include, but are not limited to, methoxy-methyl, ethoxy-methyl, 1-methoxy-ethyl, 1-ethoxy-ethyl, 2-methoxy-ethyl, 2-ethoxy-ethyl, 1-methoxy-propyl, 1-ethoxy-propyl, 2-methoxy-propyl, 2-ethoxy-propyl, 3-methoxy-propyl, 3-ethoxy-propyl, 1-methoxy-1-methyl-ethyl, 1-ethoxy-1-methyl-ethyl, 2-methoxy-1-methyl-ethyl and 2-ethoxy-1-methyl-ethyl.

In case a (C₁-C₂)alkoxy-(C₁-C₄)alkyl group is a substituent to a heterocyclyl group, the term “(C₁-C₂)alkoxy-(C₁-C₄)alkyl” means (C₁-C₂)alkoxy-(C₁-C₄)alkyl groups as defined above. Examples of said groups include, but are not limited to, methoxy-methyl, ethoxy-methyl, 1-methoxy-ethyl, 1-ethoxy-ethyl, 2-methoxy-ethyl, 2-ethoxy-ethyl, 1-methoxy-propyl, 1-ethoxy-propyl, 2-methoxy-propyl, 2-ethoxy-propyl, 3-methoxy-propyl, 3-ethoxy-propyl, 1-methoxy-1-methyl-ethyl, 1-ethoxy-1-methyl-ethyl, 2-methoxy-1-methyl-ethyl and 2-ethoxy-1-methyl-ethyl. Preferred are 2-methoxy-ethyl and 2-ethoxy-ethyl; and most preferred is 2-methoxy-ethyl.

In case “R⁹” represents “(C₁-C₂)alkoxy-(C₁-C₄)alkyl” the term means (C₁-C₂)alkoxy-(C₁-C₄)alkyl groups as defined above. Examples of said groups include, but are not limited to, methoxy-methyl, ethoxy-methyl, 1-methoxy-ethyl, 1-ethoxy-ethyl, 2-methoxy-ethyl, 2-ethoxy-ethyl, 1-methoxy-propyl, 1-ethoxy-propyl, 2-methoxy-propyl, 2-ethoxy-propyl, 3-methoxy-propyl, 3-ethoxy-propyl, 1-methoxy-1-methyl-ethyl, 1-ethoxy-1-methyl-ethyl, 2-methoxy-1-methyl-ethyl and 2-ethoxy-1-methyl-ethyl. Preferred are 2-methoxy-ethyl and 2-ethoxy-ethyl; and most preferred is 2-methoxy-ethyl.

The term “(C₃-C₆)cycloalkyl”, used alone or in combination, means a cycloalkyl group with 3 to 6 carbon atoms. Examples of (C₃-C₆)cycloalkyl groups are cyclopropyl, cyclobutyl, cyclopentyl and cyclohexyl.

The term “(C₃-C₆)cycloalkyl-(C₁-C₄)alkyl” refers to an alkyl group as defined before containing from one to four carbon atoms in which one hydrogen atom has been replaced with (C₃-C₆)cycloalkyl as defined before. Examples of (C₃-C₆)cycloalkyl-(C₁-C₄)alkyl groups include, but are not limited to, cyclopropyl-methyl, cyclobutyl-methyl, cyclopentyl-methyl, cyclohexyl-methyl, 1-cyclopropyl-ethyl, 1-cyclobutyl-ethyl, 1-cyclopentyl-ethyl, 1-cyclohexyl-ethyl, 2-cyclopropyl-ethyl, 2-cyclobutyl-ethyl, 2-cyclopentyl-ethyl, 2-cyclohexyl-ethyl, 3-cyclopropyl-propyl, 3-cyclobutyl-propyl, 3-cyclopentyl-propyl and 3-cyclohexyl-propyl. Preferred are cyclopropyl-methyl, cyclobutyl-methyl, cyclopentyl-methyl and cyclohexyl-methyl and most preferred is cyclopentyl-methyl.

The term “phenyl-(C₁-C₄)alkyl” refers to an alkyl group as defined before containing from one to four carbon atoms in which one hydrogen atom has been replaced with phenyl. Examples of phenyl-(C₁-C₄)alkyl groups include, but are not limited to, phenyl-methyl (benzyl), 1-phenyl-ethyl, 2-phenyl-ethyl, 1-phenyl-propyl, 2-phenyl-propyl, 3-phenyl-propyl, 1-phenyl-1-methyl-ethyl and 2-phenyl-1-methyl-ethyl. Preferred are benzyl and 2-phenyl-ethyl and most preferred is benzyl.

The term “phenyloxy-(C₁-C₄)alkyl” refers to an alkyl group as defined before containing from one to four carbon atoms in which one hydrogen atom has been replaced with phenyloxy (or in an alternative phrase: phenoxy). Examples of phenyloxy-(C₁-C₄)alkyl groups include, but are not limited to, phenyloxy-methyl, 1-phenyloxy-ethyl, 2-phenyloxy-ethyl, 1-phenyloxy-propyl, 2-phenyloxy-propyl, 3-phenyloxy-propyl, 1-phenyloxy-1-methyl-ethyl and 2-phenyloxy-1-methyl-ethyl. Preferred are phenyloxy-methyl and 2-phenyloxy-ethyl and most preferred is 2-phenyloxy-ethyl.

The term halogen means fluoro, chloro, bromo or iodo, preferably fluoro or chloro and most preferably fluoro.

The term “5-membered heteroaryl”, used alone or in combination, means a 5-membered monocyclic aromatic ring containing 1, 2 or 3 heteroatoms independently selected from oxygen, nitrogen and sulphur (and preferably containing 1 or 2 nitrogen atoms). Examples of such 5-membered heteroaryl groups are furanyl, oxazolyl, isoxazolyl, oxadiazolyl, thienyl, thiazolyl, isothiazolyl, thiadiazolyl, pyrrolyl, imidazolyl, pyrazolyl and triazolyl. Preferred are pyrrolyl, imidazolyl and pyrazolyl and most preferred is pyrazolyl (notably pyrazol-3-yl). The above-mentioned 5-membered heteroaryl groups are unsubstituted or mono- or di-substituted with (C₁-C₄)alkyl (preferably methyl). A preferred example of such unsubstituted or mono- or di-substituted 5-membered heteroaryl groups is 2-methyl-2H-pyrazol-3-yl.

The term “6-membered heteroaryl”, used alone or in combination, means a 6-membered monocyclic aromatic ring containing 1 or 2 nitrogen atoms. Examples of such 6-membered heteroaryl groups are pyridyl, pyrimidyl, pyridazinyl and pyrazinyl. Preferred is pyridyl. The above-mentioned 6-membered heteroaryl groups are unsubstituted or mono- or di-substituted, wherein the substituents are independently selected from the group consisting of halogen, hydroxy, (C₁-C₄)alkyl, (C₁-C₄)alkoxy, (C₁-C₄)alkylthio, (C₁-C₄)alkyl-sulfonyl, (C₁-C₄)alkyl-amino and di-[(C₁-C₄)alkyl]-amino. Preferably the substituents are independently selected from the group consisting of halogen (notably fluoro or chloro), hydroxy and (C₁-C₄)alkoxy (notably methoxy). Examples of such unsubstituted or mono- or di-substituted 6-membered heteroaryl groups are pyridin-2-yl, 4-fluoro-pyridin-2-yl, 6-fluoro-pyridin-2-yl, 6-chloro-pyridin-2-yl, 6-hydroxy-pyridin-2-yl, 6-methoxy-pyridin-2-yl, 6-methylamino-pyridin-2-yl, 6-dimethylamino-pyridin-2-yl, pyridin-3-yl, pyridin-4-yl, 2-chloro-pyridin-4-yl, pyrimidin-2-yl, 4-hydroxy-pyrimidin-2-yl, 4-methoxy-pyrimidin-2-yl, 4-methylthio-pyrimidin-2-yl, pyrimidin-4-yl, 2-chloro-pyrimidin-4-yl, 2,6-dichloro-pyrimidin-4-yl, 2-hydroxy-pyrimidin-4-yl, 6-hydroxy-pyrimidin-4-yl, 6-chloro-2-hydroxy-pyrimidin-4-yl, 2-methoxy-pyrimidin-4-yl, 6-methoxy-pyrimidin-4-yl, 6-chloro-2-methoxy-pyrimidin-4-yl, 2-methylthio-pyrimidin-4-yl, 6-methylthio-pyrimidin-4-yl, 2-methylamino-pyrimidin-4-yl, 2-dimethylamino-pyrimidin-4-yl, 6-methylsulfonyl-pyrimidin-4-yl, pyrimidin-5-yl, pyridazin-3-yl, 6-chloro-pyridazin-3-yl, 6-hydroxy-pyridazin-3-yl, 5-methoxy-pyridazin-3-yl, 6-methoxy-pyridazin-3-yl, pyrazin-2-yl, 3-chloro-pyrazin-2-yl, 5-chloro-pyrazin-2-yl, 6-chloro-pyrazin-2-yl, 6-hydroxy-pyrazin-2-yl, 5-methoxy-pyrazin-2-yl, 6-methoxy-pyrazin-2-yl, 6-methylamino-pyrazin-2-yl and 6-dimethylamino-pyrazin-2-yl. Preferred are pyridin-2-yl, 4-fluoro-pyridin-2-yl, 6-fluoro-pyridin-2-yl, 6-chloro-pyridin-2-yl, 6-hydroxy-pyridin-2-yl, 6-methoxy-pyridin-2-yl, 6-methylamino-pyridin-2-yl, pyridin-3-yl, 2-chloro-pyrimidin-4-yl, 2-hydroxy-pyrimidin-4-yl, 2-methoxy-pyrimidin-4-yl, pyridazin-3-yl, 6-chloro-pyridazin-3-yl, 6-hydroxy-pyridazin-3-yl, 6-methoxy-pyridazin-3-yl, pyrazin-2-yl, 3-chloro-pyrazin-2-yl, 6-methoxy-pyrazin-2-yl, 6-methylamino-pyrazin-2-yl and 6-dimethylamino-pyrazin-2-yl. Most preferred are pyridin-2-yl, 4-fluoro-pyridin-2-yl, 6-fluoro-pyridin-2-yl, 6-hydroxy-pyridin-2-yl, 6-methoxy-pyridin-2-yl and 2-hydroxy-pyrimidin-4-yl.

It is well known in the art that 6-membered heteroaryl groups, as defined above, may be present in different tautomeric forms (e.g. in case said heteroaryl groups are substituted with at least one hydroxy group). Examples of such tautomers are given in the formulas below:

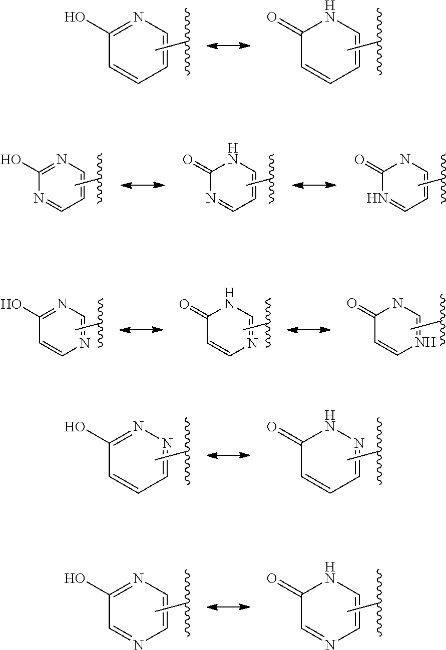

It is to be understood that in any such case all different tautomers are within the scope of the present invention. Even though one tautomer may be described, the present invention includes all tautomers of the present compounds. Especially, any given chemical name does represent not only the specifically named chemical compound but also the different tautomeric forms thereof. In solution, tautomers exist usually as mixtures of different tautomeric forms; in the solid state usually one tautomeric form predominates.

6-membered heteroaryl groups which are substituted with at least one (C₁-C₄)alkylamino group may also be present in different tautomeric forms which are all included in the present invention.

The term “heterocyclyl”, used alone or in combination, means a 6-membered monocyclic ring containing 1 or 2 double bonds (preferably 2 double bonds) and 1 or 2 nitrogen atoms, wherein one or two carbon atoms adjacent to said nitrogen atoms are substituted with an oxo-group. The heterocyclyl group may be attached to the rest of the molecule via a nitrogen atom or a carbon atom. In case Y represents —C(R⁷R⁸)—, a heterocyclyl group representing R¹ is preferably attached to the rest of the molecule via a nitrogen atom. In case Y represents —N(R⁹)—, —O—, —S—, —S(O)—, or —S(O)₂— (and notably —N(R⁹)— or —O—), a heterocyclyl group representing R¹ is preferably attached to the rest of the molecule via a carbon atom. The above-mentioned heterocyclyl groups are unsubstituted or mono- or di-substituted with (C₁-C₄)alkyl or (C₁-C₂)alkoxy-(C₁-C₄)alkyl (and preferably unsubstituted or mono-substituted with (C₁-C₄)alkyl). Preferred heterocyclyl groups are selected from the group of radicals as depicted in groups G1 and/or G2 below:

G1: heterocyclyl groups, attached to the rest of the molecule via a nitrogen atom (as depicted by the arrow):

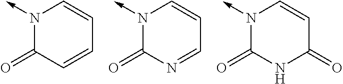

G2: heterocyclyl groups, attached to the rest of the molecule via a carbon atom (as depicted by the arrow):

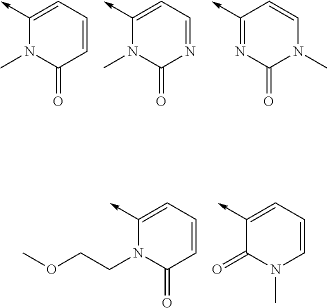

2) A further embodiment of the invention relates to benzamide derivatives according to embodiment 1), wherein
n represents 1, 2, 3 or 4 (and preferably 2, 3 or 4);
Y represents —C(R⁷R⁸)—, —N(R⁹)—, —O—, —S—, —S(O)—, or —S(O)₂—;
R¹ represents
a 5-membered heteroaryl group which is unsubstituted or mono- or di-substituted with (C₁-C₄)alkyl;
a 6-membered heteroaryl group which is unsubstituted or mono- or di-substituted, wherein the substituents are independently selected from the group consisting of halogen, hydroxy, (C₁-C₄)alkyl, (C₁-C₄)alkoxy, (C₁-C₄)alkylthio, (C₁-C₄)alkyl-sulfonyl, (C₁-C₄)alkyl-amino and di-[(C₁-C₄)alkyl]-amino;
a phenyl group which is unsubstituted or mono- or di-substituted with halogen; or
a heterocyclyl group which is unsubstituted or mono- or di-substituted with (C₁-C₄)alkyl or (C₁-C₂)alkoxy-(C₁-C₄)alkyl;
   R² represents chloro or methyl (and preferably chloro);
   R³ represents hydrogen and R⁴ represents hydroxy, hydroxy-(C₁-C₄)alkyl, —CONH₂ or (C₁-C₄)alkoxy (and preferably hydroxy, hydroxymethyl or methoxy); or
   R³ represents (C₁-C₄)alkyl or hydroxy-(C₁-C₄)alkyl (and preferably methyl or hydroxymethyl) and R⁴ represents hydrogen;
   R⁵ represents hydrogen or fluoro;
   R⁶ represents hydrogen or fluoro;
   R⁷ and R⁸ represent independently from each other hydrogen or fluoro; or R⁷ and R⁸ together represent an oxo-group; and
   R⁹ represents hydrogen, (C₁-C₄)alkyl, (C₁-C₂)alkoxy-(C₁-C₄)alkyl, (C₃-C₆)cycloalkyl-(C₁-C₄)alkyl, phenyl-(C₁-C₄)alkyl, or phenyloxy-(C₁-C₄)alkyl;
   and to the salts (in particular pharmaceutically acceptable salts) of such compounds.
   3) A further embodiment of the invention relates to benzamide derivatives according to embodiment 1), wherein
   n represents 2, 3 or 4;
   Y represents —C(R⁷R⁸)—, —N(R⁹)—, —O—, or —S—;
   R¹ represents a 6-membered heteroaryl group which is unsubstituted or mono- or di-substituted, wherein the substituents are independently selected from the group consisting of halogen, hydroxy, (C₁-C₄)alkoxy, (C₁-C₄)alkylthio, (C₁-C₄)alkyl-sulfonyl, (C₁-C₄)alkyl-amino and di-[(C₁-C₄)alkyl]-amino (preferably halogen, hydroxy and (C₁-C₄)alkoxy);
   R² represents chloro;
   R³ represents hydrogen and R⁴ represents hydroxy or hydroxy-(C₁-C₄)alkyl (and preferably hydroxy or hydroxymethyl); or
   R³ represents (C₁-C₄)alkyl or hydroxy-(C₁-C₄)alkyl (and preferably methyl or hydroxymethyl) and R⁴ represents hydrogen;
   R⁵ represents hydrogen or fluoro;
   R⁶ represents hydrogen or fluoro;
   R⁷ and R⁸ represent independently from each other hydrogen, fluoro, hydroxy or (C₁-C₄)alkyl, with the proviso that R⁸ is different from fluoro or hydroxy if R⁷ represents hydroxy;
   or R⁷ and R⁸ together represent an oxo-group; and
   R⁹ represents hydrogen, (C₁-C₄)alkyl, (C₁-C₂)alkoxy-(C₁-C₄)alkyl, (C₃-C₆)cycloalkyl-(C₁-C₄)alkyl, phenyl-(C₁-C₄)alkyl, or phenyloxy-(C₁-C₄)alkyl (and preferably hydrogen or (C₁-C₄)alkyl); and to the salts (in particular pharmaceutically acceptable salts) of such compounds.
   4) A further embodiment of the invention relates to benzamide derivatives according to any one of embodiments 1) or 2), wherein
   n represents 2, 3 or 4;
   Y represents —C(R⁷R⁸)—, —N(R⁹)—, —O—, or —S—;
   R¹ represents a 6-membered heteroaryl group which is unsubstituted or mono- or di-substituted, wherein the substituents are independently selected from the group consisting of halogen, hydroxy, (C₁-C₄)alkoxy, (C₁-C₄)alkylthio, (C₁-C₄)alkyl-sulfonyl, (C₁-C₄)alkyl-amino and di-[(C₁-C₄)alkyl]-amino (preferably halogen, hydroxy and (C₁-C₄)alkoxy);
   R² represents chloro;
   R³ represents hydrogen and R⁴ represents hydroxy or hydroxy-(C₁-C₄)alkyl (and preferably hydroxy or hydroxymethyl); or
   R³ represents (C₁-C₄)alkyl or hydroxy-(C₁-C₄)alkyl (and preferably methyl or hydroxymethyl) and R⁴ represents hydrogen;
   R⁵ represents hydrogen or fluoro;
   R⁶ represents hydrogen or fluoro;
   R⁷ and R⁸ represent independently from each other hydrogen or fluoro; or R⁷ and R⁸ together represent an oxo-group; and
   R⁹ represents hydrogen, (C₁-C₄)alkyl, (C₁-C₂)alkoxy-(C₁-C₄)alkyl, (C₃-C₆)cycloalkyl-(C₁-C₄)alkyl, phenyl-(C₁-C₄)alkyl, or phenyloxy-(C₁-C₄)alkyl (and preferably hydrogen or (C₁-C₄)alkyl);
   and to the salts (in particular pharmaceutically acceptable salts) of such compounds.
   5) A further embodiment of the invention relates to benzamide derivatives according to any one of embodiments 1) or 2), wherein
   n represents 1, 2, 3 or 4 (and preferably 2, 3 or 4);
   Y represents —C(R⁷R⁸)—, —N(R⁹)—, —O—, —S—, —S(O)—, or —S(O)₂—;
   R¹ represents
a 5-membered heteroaryl group which is unsubstituted or mono- or di-substituted (preferably mono-substituted) with (C₁-C₄)alkyl;
a 6-membered heteroaryl group which is unsubstituted or mono- or di-substituted, wherein the substituents are independently selected from the group consisting of halogen, hydroxy, (C₁-C₄)alkyl, (C₁-C₄)alkoxy, (C₁-C₄)alkylthio, (C₁-C₄)alkyl-sulfonyl, (C₁-C₄)alkyl-amino and di-[(C₁-C₄)alkyl]-amino;
a phenyl group which is unsubstituted or mono- or di-substituted with halogen; or
a heterocyclyl group which is unsubstituted or mono- or di-substituted (preferably unsubstituted or mono-substituted) with (C₁-C₄)alkyl or (C₁-C₂)alkoxy-(C₁-C₄)alkyl;
   R² represents chloro or methyl (and preferably chloro);
   R³ represents hydrogen and R⁴ represents hydroxy, hydroxy-(C₁-C₄)alkyl, —CONH₂ or (C₁-C₄)alkoxy (and preferably hydroxy, hydroxymethyl or methoxy);
   R⁵ represents hydrogen or fluoro;
   R⁶ represents hydrogen or fluoro;
   R⁷ and R⁸ represent independently from each other hydrogen or fluoro; or R⁷ and R⁸ together represent an oxo-group; and
   R⁹ represents hydrogen, (C₁-C₄)alkyl, (C₁-C₂)alkoxy-(C₁-C₄)alkyl, (C₃-C₆)cycloalkyl-(C₁-C₄)alkyl, phenyl-(C₁-C₄)alkyl, or phenyloxy-(C₁-C₄)alkyl;
   and to the salts (in particular pharmaceutically acceptable salts) of such compounds.
   6) A further embodiment of the invention relates to benzamide derivatives according to any one of embodiments 1) or 2), wherein
   n represents 2, 3 or 4;
   Y represents —C(R⁷R⁸)—, —N(R⁹)—, —O—, or —S—;
   R¹ represents a 6-membered heteroaryl group which is unsubstituted or mono- or di-substituted (preferably unsubstituted or mono-substituted), wherein the substituents are independently selected from the group consisting of halogen, hydroxy, (C₁-C₄)alkoxy, (C₁-C₄)alkylthio, (C₁-C₄)alkyl-sulfonyl, (C₁-C₄)alkyl-amino and di-[(C₁-C₄)alkyl]-amino (preferably halogen, hydroxy and (C₁-C₄)alkoxy);
   R² represents chloro;
   R³ represents hydrogen and R⁴ represents hydroxy or hydroxy-(C₁-C₄)alkyl (and preferably hydroxy or hydroxymethyl);
   R⁵ represents hydrogen or fluoro;
   R⁶ represents hydrogen or fluoro;
   R⁷ and R⁸ represent independently from each other hydrogen or fluoro; or R⁷ and R⁸ together represent an oxo-group; and
   R⁹ represents hydrogen, (C₁-C₄)alkyl, (C₁-C₂)alkoxy-(C₁-C₄)alkyl, (C₃-C₆)cycloalkyl-(C₁-C₄)alkyl, phenyl-(C₁-C₄)alkyl, or phenyloxy-(C₁-C₄)alkyl (and preferably hydrogen or (C₁-C₄)alkyl);
   and to the salts (in particular pharmaceutically acceptable salts) of such compounds.
   7) A further embodiment of the invention relates to benzamide derivatives according to any one of embodiments 1) or 2), wherein
   n represents 1, 2, 3 or 4 (and preferably 2, 3 or 4);
   Y represents —C(R⁷R⁸)—, —N(R⁹)—, —O—, or —S-(preferably —N(R⁹)—);
   R¹ represents a 6-membered heteroaryl group which is unsubstituted or mono- or di-substituted, wherein the substituents are independently selected from the group consisting of halogen, hydroxy, (C₁-C₄)alkyl, (C₁-C₄)alkoxy, (C₁-C₄)alkylthio, (C₁-C₄)alkyl-sulfonyl, (C₁-C₄)alkyl-amino and di-[(C₁-C₄)alkyl]-amino (preferably halogen, hydroxy and (C₁-C₄)alkylthio);
   R² represents chloro;
   R³ represents (C₁-C₄)alkyl or hydroxy-(C₁-C₄)alkyl (and preferably methyl or hydroxymethyl) and R⁴ represents hydrogen;
   R⁵ represents hydrogen or fluoro (preferably hydrogen);
   R⁶ represents hydrogen or fluoro (preferably hydrogen);
   R⁷ and R⁸ represent independently from each other hydrogen or fluoro; or R⁷ and R⁸ together represent an oxo-group; and
   R⁹ represents hydrogen, (C₁-C₄)alkyl, (C₁-C₂)alkoxy-(C₁-C₄)alkyl, (C₃-C₆)cycloalkyl-(C₁-C₄)alkyl, phenyl-(C₁-C₄)alkyl, or phenyloxy-(C₁-C₄)alkyl (preferably hydrogen or (C₁-C₄)alkyl);
   and to the salts (in particular pharmaceutically acceptable salts) of such compounds.
   8) A further embodiment of the invention relates to benzamide derivatives according to any one of embodiments 1) or 2), wherein
   n represents 2;
   Y represents —N(R⁹)—;
   R¹ represents a 6-membered heteroaryl group which is unsubstituted or mono- or di-substituted (preferably mono-substituted), wherein the substituents are independently selected from the group consisting of halogen, hydroxy and (C₁-C₄)alkylthio (preferably halogen and hydroxy);
   R² represents chloro;
   R³ represents (C₁-C₄)alkyl or hydroxy-(C₁-C₄)alkyl (and preferably methyl or hydroxymethyl) and R⁴ represents hydrogen;
   R⁵ represents hydrogen;
   R⁶ represents hydrogen; and
   R⁹ represents hydrogen or (C₁-C₄)alkyl (and preferably hydrogen or methyl); and to the salts (in particular pharmaceutically acceptable salts) of such compounds.
   9) A further embodiment of the invention relates to benzamide derivatives according to any one of embodiments 1) to 7), wherein
   n represents 2, 3 or 4 (preferably 2 or 3 and most preferably 2);
   and to the salts (in particular pharmaceutically acceptable salts) of such compounds.
   10) A further embodiment of the invention relates to benzamide derivatives according to any one of embodiments 1) to 7), wherein
   n represents 3 or 4;
   and to the salts (in particular pharmaceutically acceptable salts) of such compounds.
   11) A further embodiment of the invention relates to benzamide derivatives according to any one of embodiments 1) to 7), 9) or 10), wherein
   Y represents —C(R⁷R⁸)—, —N(R⁹)—, —O—, or —S—; and to the salts (in particular pharmaceutically acceptable salts) of such compounds.
   12) A further embodiment of the invention relates to benzamide derivatives according to any one of embodiments 1) to 7), 9) or 10), wherein
   Y represents —C(R⁷R⁸)—, —O—, or —S—;
   and to the salts (in particular pharmaceutically acceptable salts) of such compounds.
   13) A further embodiment of the invention relates to benzamide derivatives according to any one of embodiments 1) to 7), 9) or 10), wherein
   Y represents —C(R⁷R⁸)—;
   and to the salts (in particular pharmaceutically acceptable salts) of such compounds.
   14) A further embodiment of the invention relates to benzamide derivatives according to any one of embodiments 1) to 10), wherein
   Y represents —N(R⁹)—;
   and to the salts (in particular pharmaceutically acceptable salts) of such compounds.
   15) A further embodiment of the invention relates to benzamide derivatives according to any one of embodiments 1) to 7), 9) or 10), wherein
   Y represents —O—;
   and to the salts (in particular pharmaceutically acceptable salts) of such compounds.
   16) A further embodiment of the invention relates to benzamide derivatives according to any one of embodiments 1) to 7), 9) or 10), wherein
   Y represents —S—, —S(O)—, or —S(O)₂— (preferably —S—);
   and to the salts (in particular pharmaceutically acceptable salts) of such compounds.
   17) A further embodiment of the invention relates to benzamide derivatives according to any one of embodiments 1), 2), 5) or 9) to 16), wherein
   R¹ represents
a 5-membered heteroaryl group which is unsubstituted or mono- or di-substituted (preferably mono-substituted) with (C₁-C₄)alkyl;
a 6-membered heteroaryl group which is unsubstituted or mono- or di-substituted, wherein the substituents are independently selected from the group consisting of halogen, hydroxy, (C₁-C₄)alkoxy, (C₁-C₄)alkylthio, (C₁-C₄)alkyl-sulfonyl, (C₁-C₄)alkyl-amino and di-[(C₁-C₄)alkyl]-amino (preferably from halogen, hydroxy and (C₁-C₄)alkoxy; and most preferably from halogen and hydroxy);
a phenyl group which is unsubstituted or mono- or di-substituted with halogen; and to the salts (in particular pharmaceutically acceptable salts) of such compounds.
   18) A further embodiment of the invention relates to benzamide derivatives according to any one of embodiments 1), 2), 5) or 9) to 16), wherein
   R¹ represents
a 5-membered heteroaryl group which is unsubstituted or mono- or di-substituted (preferably mono-substituted) with (C₁-C₄)alkyl (preferably methyl);
a 6-membered heteroaryl group which is unsubstituted or mono- or di-substituted, wherein the substituents are independently selected from the group consisting of halogen, hydroxy, (C₁-C₄)alkoxy, (C₁-C₄)alkylthio, (C₁-C₄)alkyl-sulfonyl, (C₁-C₄)alkyl-amino and di-[(C₁-C₄)alkyl]-amino (preferably from halogen, hydroxy and methoxy; and most preferably from halogen and hydroxy); and to the salts (in particular pharmaceutically acceptable salts) of such compounds.
   19) A further embodiment of the invention relates to benzamide derivatives according to any one of embodiments 1), 2), 5) or 9) to 16), wherein
   R¹ represents a 5-membered heteroaryl group which is unsubstituted or mono- or di-substituted (preferably mono-substituted) with (C₁-C₄)alkyl (preferably methyl); and to the salts (in particular pharmaceutically acceptable salts) of such compounds.
   20) A further embodiment of the invention relates to benzamide derivatives according to any one of embodiments 1) to 16), wherein
   R¹ represents a 6-membered heteroaryl group which is unsubstituted or mono- or di-substituted, wherein the substituents are independently selected from the group consisting of halogen, hydroxy, (C₁-C₄)alkyl, (C₁-C₄)alkoxy, (C₁-C₄)alkylthio, (C₁-C₄)alkyl-sulfonyl, (C₁-C₄)alkyl-amino and di-[(C₁-C₄)alkyl]-amino (preferably from halogen, hydroxy and methoxy; and most preferably from halogen and hydroxy);
   and to the salts (in particular pharmaceutically acceptable salts) of such compounds.
   21) A further embodiment of the invention relates to benzamide derivatives according to any one of embodiments 1) to 16), wherein
   R¹ represents a 6-membered heteroaryl group which is unsubstituted or mono-substituted (preferably mono-substituted), wherein the substituents are independently selected from the group consisting of halogen, hydroxy and (C₁-C₄)alkoxy (preferably from fluoro, chloro, hydroxy and methoxy; and most preferably from fluoro, chloro and hydroxy); and to the salts (in particular pharmaceutically acceptable salts) of such compounds.
   22) A further embodiment of the invention relates to benzamide derivatives according to any one of embodiments 1), 2), 5) or 9) to 16), wherein
   R¹ represents a phenyl group which is unsubstituted or mono- or di-substituted with halogen (preferably fluoro);
   and to the salts (in particular pharmaceutically acceptable salts) of such compounds.
   23) A further embodiment of the invention relates to benzamide derivatives according to any one of embodiments 1), 2), 5) or 9) to 16), wherein
   R¹ represents a heterocyclyl group which is unsubstituted or mono- or di-substituted (preferably unsubstituted or mono-substituted) with (C₁-C₄)alkyl or (C₁-C₂)alkoxy-(C₁-C₄)alkyl;
   and to the salts (in particular pharmaceutically acceptable salts) of such compounds.
   24) A further embodiment of the invention relates to benzamide derivatives according to embodiment 23), wherein
   the heterocyclyl group is selected from groups G1 and/or G2;
   and to the salts (in particular pharmaceutically acceptable salts) of such compounds.
   25) A further embodiment of the invention relates to benzamide derivatives according to any one of embodiments 1) to 24), wherein
   R² represents chloro;
   and to the salts (in particular pharmaceutically acceptable salts) of such compounds.
   26) A further embodiment of the invention relates to benzamide derivatives according to any one of embodiments 1), 2), 5) or 9) to 24), wherein
   R² represents methyl;
   and to the salts (in particular pharmaceutically acceptable salts) of such compounds.
   27) A further embodiment of the invention relates to benzamide derivatives according to any one of embodiments 1), 2), 5) or 9) to 26), wherein
   R³ represents hydrogen and R⁴ represents hydroxy, hydroxy-(C₁-C₄)alkyl, —CONH₂ or (C₁-C₄)alkoxy (and preferably hydroxy, hydroxymethyl or methoxy);
   and to the salts (in particular pharmaceutically acceptable salts) of such compounds.
   28) A further embodiment of the invention relates to benzamide derivatives according to any one of embodiments 1) to 6) or 9) to 26), wherein
   R³ represents hydrogen and R⁴ represents hydroxy or hydroxy-(C₁-C₄)alkyl (and preferably hydroxy or hydroxymethyl);
   and to the salts (in particular pharmaceutically acceptable salts) of such compounds.
   29) A further embodiment of the invention relates to benzamide derivatives according to any one of embodiments 1) to 6) or 9) to 26), wherein
   R³ represents hydrogen and R⁴ represents hydroxy;
   and to the salts (in particular pharmaceutically acceptable salts) of such compounds.
   30) A further embodiment of the invention relates to benzamide derivatives according to any one of embodiments 1) to 6) or 9) to 26), wherein
   R³ represents hydrogen and R⁴ represents hydroxy-(C₁-C₄)alkyl (preferably hydroxymethyl);
   and to the salts (in particular pharmaceutically acceptable salts) of such compounds.
   31) A further embodiment of the invention relates to benzamide derivatives according to any one of embodiments 1) to 4) or 7) to 26), wherein
   R³ represents (C₁-C₄)alkyl or hydroxy-(C₁-C₄)alkyl (and preferably methyl or hydroxymethyl) and R⁴ represents hydrogen;
   and to the salts (in particular pharmaceutically acceptable salts) of such compounds.
   32) A further embodiment of the invention relates to benzamide derivatives according to any one of embodiments 1) to 4) or 7) to 26), wherein
   R³ represents (C₁-C₄)alkyl (preferably methyl) and R⁴ represents hydrogen; and to the salts (in particular pharmaceutically acceptable salts) of such compounds.
   33) A further embodiment of the invention relates to benzamide derivatives according to any one of embodiments 1) to 4) or 7) to 26), wherein
   R³ represents hydroxy-(C₁-C₄)alkyl (preferably hydroxymethyl) and R⁴ represents hydrogen;
   and to the salts (in particular pharmaceutically acceptable salts) of such compounds.
   34) A further embodiment of the invention relates to benzamide derivatives according to any one of embodiments 1) to 33), wherein
   R⁵ and R⁶ both represent hydrogen;
   and to the salts (in particular pharmaceutically acceptable salts) of such compounds.
   35) A further embodiment of the invention relates to benzamide derivatives according to any one of embodiments 1) to 7) or 9) to 33), wherein
   R⁵ and R⁶ both represent fluoro;
   and to the salts (in particular pharmaceutically acceptable salts) of such compounds.
   36) A further embodiment of the invention relates to benzamide derivatives according to any one of embodiments 1) to 7) or 9) to 33), wherein
   R⁵ represents hydrogen and R⁶ represents fluoro; and to the salts (in particular pharmaceutically acceptable salts) of such compounds.
   37) A further embodiment of the invention relates to benzamide derivatives according to any one of embodiments 1), 3) or 9) to 36), wherein
   R⁷ and R⁸ represent independently from each other hydrogen, fluoro, hydroxy or (C₁-C₄)alkyl, with the proviso that R⁸ is different from fluoro or hydroxy if R⁷ represents hydroxy;
   and to the salts (in particular pharmaceutically acceptable salts) of such compounds.
   38) A further embodiment of the invention relates to benzamide derivatives according to any one of embodiments 1) to 7) or 9) to 36), wherein
   R⁷ and R⁸ represent independently from each other hydrogen or fluoro;
   and to the salts (in particular pharmaceutically acceptable salts) of such compounds.
   39) A further embodiment of the invention relates to benzamide derivatives according to any one of embodiments 1) to 7) or 9) to 36), wherein
   R⁷ and R⁸ both represent hydrogen;
   and to the salts (in particular pharmaceutically acceptable salts) of such compounds.
   40) A further embodiment of the invention relates to benzamide derivatives according to any one of embodiments 1) to 7) or 9) to 36), wherein
   R⁷ and R⁸ both represent fluoro;
   and to the salts (in particular pharmaceutically acceptable salts) of such compounds.
   41) A further embodiment of the invention relates to benzamide derivatives according to any one of embodiments 1) to 7) or 9) to 36), wherein
   R⁷ represents hydrogen and R⁸ represents fluoro;
   and to the salts (in particular pharmaceutically acceptable salts) of such compounds.
   42) A further embodiment of the invention relates to benzamide derivatives according to any one of embodiments 1), 3) or 9) to 36), wherein
   R⁷ represents hydrogen or (C₁-C₄)alkyl (preferably (C₁-C₄)alkyl) and R⁸ represents hydroxy;
   and to the salts (in particular pharmaceutically acceptable salts) of such compounds.
   43) A further embodiment of the invention relates to benzamide derivatives according to any one of embodiments 1) to 7) or 9) to 36), wherein
   R⁷ and R⁸ together represent an oxo-group;
   and to the salts (in particular pharmaceutically acceptable salts) of such compounds.
   44) A further embodiment of the invention relates to benzamide derivatives according to any one of embodiments 1) to 7) or 9) to 43), wherein
   R⁹ represents hydrogen, methyl, ethyl, n-propyl, iso-propyl, iso-butyl, 2-methoxy-ethyl, cyclopentyl-methyl, benzyl, or 2-phenyloxy-ethyl;
   and to the salts (in particular pharmaceutically acceptable salts) of such compounds.
   45) A further embodiment of the invention relates to benzamide derivatives according to any one of embodiments 1) to 43), wherein
   R⁹ represents hydrogen or (C₁-C₄)alkyl (preferably methyl, ethyl, n-propyl, iso-propyl or iso-butyl);
   and to the salts (in particular pharmaceutically acceptable salts) of such compounds.
   46) A further embodiment of the invention relates to benzamide derivatives according to any one of embodiments 1) to 7), 9) to 38), 41), 42) or 44) to 45), wherein,
   in case R⁷ and R⁹ are different from each other, the carbon atom of the group —C(R⁷R⁹)— has (S)-configuration;
   and to the salts (in particular pharmaceutically acceptable salts) of such compounds.
   47) A further embodiment of the invention relates to benzamide derivatives according to any one of embodiments 1) to 7), 9) to 38), 41), 42) or 44) to 45), wherein,
   in case R⁷ and R⁹ are different from each other, the carbon atom of the group —C(R⁷R⁹)— has (R)-configuration;
   and to the salts (in particular pharmaceutically acceptable salts) of such compounds.
   48) A further embodiment of the invention relates to benzamide derivatives according to any one of embodiments 1) to 4), 7) to 26) or 31) to 47), wherein
   the carbon atom, which is attached to the group R³, has (S)-configuration;
   and to the salts (in particular pharmaceutically acceptable salts) of such compounds.
   49) A further embodiment of the invention relates to benzamide derivatives according to any one of embodiments 1) to 4), 7) to 26) or 31) to 47), wherein the carbon atom, which is attached to the group R³, has (R)-configuration;
   and to the salts (in particular pharmaceutically acceptable salts) of such compounds.
   50) A further embodiment of the invention relates to benzamide derivatives according to any one of embodiments 1) to 7), 9) to 33) or 35) to 49), wherein,
   in case n is different from 2 and at least one of R⁵ and R⁶ is different from hydrogen, the carbon atom, which is attached to the group R⁴, has (S)-configuration;
   and to the salts (in particular pharmaceutically acceptable salts) of such compounds.
   51) A further embodiment of the invention relates to benzamide derivatives according to any one of embodiments 1) to 7), 9) to 33) or 35) to 49), wherein,
   in case n is different from 2 and at least one of R⁵ and R⁶ is different from hydrogen, the carbon atom, which is attached to the group R⁴, has (R)-configuration; and to the salts (in particular pharmaceutically acceptable salts) of such compounds.
   52) A further embodiment of the invention relates to benzamide derivatives according to any one of embodiments 1) to 7), 9) to 33) or 35) to 51), wherein,
   in case n is different from 2 and one of R⁵ or R⁶ represents fluoro, the carbon atom, which
   is attached to the groups R⁵ and R⁶, has (S)-configuration; and to the salts (in particular pharmaceutically acceptable salts) of such compounds.
   53) A further embodiment of the invention relates to benzamide derivatives according to any one of embodiments 1) to 7), 9) to 33) or 35) to 51), wherein,
   in case n is different from 2 and one of R⁵ or R⁶ represents fluoro, the carbon atom, which is attached to the groups R⁵ and R⁶, has (R)-configuration;
   and to the salts (in particular pharmaceutically acceptable salts) of such compounds.
   54) Preferred compounds of formula (I) as defined in embodiment 1) are selected from the group consisting of:
2-Chloro-N-(1-hydroxy-cyclohexylmethyl)-5-phenylamino-benzamide;
2-Chloro-N-((1-hydroxycyclohexyl)methyl)-5-(methyl(phenyl)amino)benzamide;
2-Chloro-N-(1-hydroxy-cyclohexyl methyl)-5-[(2-methoxy-ethyl)-phenyl-amino]-benzamide;
2-Chloro-5-(2-fluoro-phenylamino)-N-(1-hydroxy-cyclohexylmethyl)-benzamide;
2-Chloro-5-[(2-fluoro-phenyl)-methyl-amino]-N-(1-hydroxy-cyclohexylmethyl)-benzamide;
2-Chloro-5-(2,4-difluoro-phenylamino)-N-(1-hydroxy-cyclohexylmethyl)-benzamide;
2-Chloro-5-[(2,4-difluoro-phenyl)-methyl-amino]-N-(1-hydroxy-cyclohexylmethyl)-benzamide;
2-Chloro-N-(1-hydroxy-cyclohexylmethyl)-5-(pyrimidin-4-ylamino)-benzamide;
2-Chloro-N-(1-hydroxy-cyclohexylmethyl)-5-(methyl-pyrimidin-4-yl-amino)-benzamide;
2-Chloro-5-(2-chloro-pyrimidin-4-ylamino)-N-(1-hydroxy-cyclohexylmethyl)-benzamide;
2-Chloro-5-[(2-chloro-pyrimidin-4-yl)-methyl-amino]-N-(1-hydroxy-cyclohexylmethyl)-benzamide;
2-Chloro-N-(1-hydroxy-cyclohexylmethyl)-5-(2-methoxy-pyrimidin-4-ylamino)-benzamide;
2-Chloro-N-(1-hydroxy-cyclohexylmethyl)-5-[(2-methoxy-pyrimidin-4-yl)-methyl-amino]-benzamide;
2-Chloro-N-(1-hydroxy-cyclohexylmethyl)-5-[methyl-(2-methylamino-pyrimidin-4-yl)-amino]-benzamide;
2-Chloro-5-[(2-dimethylamino-pyrimidin-4-yl)-methyl-amino]-N-(1-hydroxy-cyclohexylmethyl)-benzamide;
2-Chloro-N-(1-hydroxy-cyclohexylmethyl)-5-(2-hydroxypyrimidin-4-ylamino)-benzamide;
2-Chloro-N-(1-hydroxy-cyclohexylmethyl)-5-[methyl-(2-oxo-1,2-dihydro-pyrimidin-4-yl)-amino]-benzamide;
2-Chloro-N-(1-hydroxy-cyclohexylmethyl)-5-[methyl-(3-methyl-2-oxo-2,3-dihydro-pyrimidin-4-yl)-amino]-benzamide;
2-Chloro-N-(1-hydroxy-cyclohexylmethyl)-5-[methyl-(1-methyl-2-oxo-1,2-dihydro-pyrimidin-4-yl)-amino]-benzamide;
2-Chloro-5-[(2-chloro-pyrimidin-4-yl)-ethyl-amino]-N-(1-hydroxy-cyclohexylmethyl)-benzamide;
2-Chloro-5-[ethyl-(2-oxo-1,2-dihydro-pyrimidin-4-yl)-amino]-N-(1-hydroxy-cyclohexylmethyl)-benzamide;
2-Chloro-5-[(2-chloro-pyrimidin-4-yl)-propyl-amino]-N-(1-hydroxy-cyclohexylmethyl)-benzamide;
2-Chloro-N-(1-hydroxy-cyclohexylmethyl)-5-[(2-oxo-1,2-dihydro-pyrimidin-4-yl)-propyl-amino]-benzamide;
2-Chloro-5-[(2-chloro-pyrimidin-4-yl)-isopropyl-amino]-N-(1-hydroxy-cyclohexylmethyl)-benzamide;
2-Chloro-N-(1-hydroxy-cyclohexylmethyl)-5-[isopropyl-(2-oxo-1,2-dihydro-pyrimidin-4-yl)-amino]-benzamide;
2-Chloro-5-[(2-chloro-pyrimidin-4-yl)-isobutyl-amino]-N-(1-hydroxy-cyclohexylmethyl)-benzamide;
2-Chloro-N-(1-hydroxy-cyclohexylmethyl)-5-[isobutyl-(2-oxo-1,2-dihydro-pyrimidin-4-yl)-amino]-benzamide;
2-Chloro-N-(1-hydroxy-cyclohexylmethyl)-5-[isobutyl-(2-methoxy-pyrimidin-4-yl)-amino]-benzamide;
5-[Benzyl-(2-chloro-pyrimidin-4-yl)-amino]-2-chloro-N-(1-hydroxy-cyclohexylmethyl)-benzamide;
5-[Benzyl-(2-oxo-1,2-dihydro-pyrimidin-4-yl)-amino]-2-chloro-N-(1-hydroxy-cyclohexylmethyl)-benzamide;
5-[Benzyl-(2-methoxy-pyrimidin-4-yl)-amino]-2-chloro-N-(1-hydroxy-cyclohexylmethyl)-benzamide;
2-Chloro-5-[(2-chloro-pyrimidin-4-yl)-(2-methoxy-ethyl)-amino]-N-(1-hydroxy-cyclohexylmethyl)-benzamide;
2-Chloro-N-(1-hydroxy-cyclohexylmethyl)-5-[(2-methoxy-ethyl)-(2-oxo-1,2-dihydro-pyrimidin-4-yl)-amino]-benzamide;
2-Chloro-N-(1-hydroxy-cyclohexylmethyl)-5-[(2-methoxy-ethyl)-(2-methoxy-pyrimidin-4-yl)-amino]-benzamide;
2-Chloro-5-[(2-chloro-pyrimidin-4-yl)-cyclopentylmethyl-amino]-N-(1-hydroxy-cyclohexylmethyl)-benzamide;
2-Chloro-5-[cyclopentylmethyl-(2-oxo-1,2-dihydro-pyrimidin-4-yl)-amino]-N-(1-hydroxy-cyclohexylmethyl)-benzamide;
2-Chloro-5-[cyclopentylmethyl-(2-methoxy-pyrimidin-4-yl)-amino]-N-(1-hydroxy-cyclohexylmethyl)-benzamide;
2-Chloro-5-[(2-chloro-pyrimidin-4-yl)-(2-phenoxy-ethyl)-amino]-N-(1-hydroxy-cyclohexylmethyl)-benzamide;
2-Chloro-N-(1-hydroxy-cyclohexylmethyl)-5-[(2-oxo-1,2-dihydro-pyrimidin-4-yl)-(2-phenoxy-ethyl)-amino]-benzamide;
2-Chloro-N-(1-hydroxy-cyclohexylmethyl)-5-[(2-methoxy-pyrimidin-4-yl)-(2-phenoxy-ethyl)-amino]-benzamide;
2-Chloro-5-[(2-chloro-pyrimidin-4-yl)-methyl-amino]-N—((R)-1-cyclohexyl-ethyl)-benzamide;
2-Chloro-N—((R)-1-cyclohexyl-ethyl)-5-[methyl-(2-oxo-1,2-dihydro-pyrimidin-4-yl)-amino]-benzamide;
2-Chloro-5-[(2-chloro-pyrimidin-4-yl)-methyl-amino]-N—((S)-1-cyclohexyl-2-hydroxy-ethyl)-benzamide;
2-Chloro-N—((S)-1-cyclohexyl-2-hydroxy-ethyl)-5-[methyl-(2-oxo-1,2-dihydro-pyrimidin-4-yl)-amino]-benzamide;
2-Chloro-5-[(2-chloro-pyrimidin-4-yl)-methyl-amino]-N-(1-hydroxy-cycloheptylmethyl)-benzamide;
2-Chloro-N-(1-hydroxy-cycloheptylmethyl)-5-[methyl-(2-oxo-1,2-dihydro-pyrimidin-4-yl)-amino]-benzamide;
2-Chloro-5-[(2-chloro-pyrimidin-4-yl)-methyl-amino]-N-(1-hydroxy-cyclooctylmethyl)-benzamide;
2-Chloro-N-(1-hydroxy-cyclooctylmethyl)-5-[methyl-(2-oxo-2,3-dihydro-pyrimidin-4-yl)-amino]-benzamide;
2-Chloro-5-[(2-chloro-pyrimidin-4-yl)-methyl-amino]-N-(1-hydroxy-cyclopentylmethyl)-benzamide;
2-Chloro-N-(1-hydroxy-cyclopentylmethyl)-5-[methyl-(2-oxo-2,3-dihydro-pyrimidin-4-yl)-amino]-benzamide;
2-Chloro-5-[(2-chloro-pyrimidin-4-yl)-methyl-amino]-N-(1-methoxy-cyclohexylmethyl)-benzamide;
2-Chloro-N-(1-methoxy-cyclohexylmethyl)-5-[methyl-(2-oxo-2,3-dihydro-pyrimidin-4-yl)-amino]-benzamide;
N-(1-Carbamoyl-cyclohexylmethyl)-2-chloro-5-[(2-chloro-pyrimidin-4-yl)-methyl-amino]-benzamide;
N-(1-Carbamoyl-cyclohexylmethyl)-2-chloro-5-[methyl-(2-oxo-2,3-dihydro-pyrimidin-4-yl)-amino]-benzamide;
2-Chloro-5-[(2-chloro-pyrimidin-4-yl)-methyl-amino]-N-(1-hydroxymethyl-cyclohexylmethyl)-benzamide;
2-Chloro-N-(1-hydroxymethyl-cyclohexylmethyl)-5-[methyl-(2-oxo-2,3-dihydro-pyrimidin-4-yl)-amino]-benzamide;
2-Chloro-5-[(2-chloro-pyrimidin-4-yl)-methyl-amino]-N-(4,4-difluoro-1-hydroxy-cyclohexylmethyl)-benzamide;
2-Chloro-N-(4,4-difluoro-1-hydroxy-cyclohexylmethyl)-5-[methyl-(2-oxo-2,3-dihydro-pyrimidin-4-yl)-amino]-benzamide;
2-Chloro-N—((R)-1-cyclohexyl-ethyl)-5-(2-methylsulfanyl-pyrimidin-4-ylamino)-benzamide;
2-Chloro-N—((S)-1-cyclohexyl-2-hydroxy-ethyl)-5-(2-methylsulfanyl-pyrimidin-4-ylamino)-benzamide;
2-Chloro-N-(1-hydroxy-cycloheptylmethyl)-5-(2-methylsulfanyl-pyrimidin-4-ylamino)-benzamide;
2-Chloro-5-[(2,6-dichloro-pyrimidin-4-yl)-methyl-amino]-N-(1-hydroxy-cyclohexylmethyl)-benzamide;
2-Chloro-5-[(6-chloro-2-methoxy-pyrimidin-4-yl)-methyl-amino]-N-(1-hydroxy-cyclohexylmethyl)-benzamide;
2-Chloro-5-[(6-chloro-2-oxo-1,2-dihydro-pyrimidin-4-yl)-methyl-amino]-N-(1-hydroxy-cyclohexylmethyl)-benzamide;
2-Chloro-N-(1-hydroxy-cyclohexylmethyl)-5-[methyl-(6-methylsulfanyl-pyrimidin-4-yl)-amino]-benzamide;
2-Chloro-N-(1-hydroxy-cyclohexylmethyl)-5-[(6-methanesulfonyl-pyrimidin-4-yl)-methyl-amino]-benzamide;
2-Chloro-N-(1-hydroxy-cyclohexylmethyl)-5-[(6-methoxy-pyrimidin-4-yl)-methyl-amino]-benzamide;
2-Chloro-N-(1-hydroxy-cyclohexylmethyl)-5-[methyl-(6-oxo-1,6-dihydro-pyrimidin-4-yl)-amino]-benzamide;
2-Chloro-N-(1-hydroxy-cyclohexylmethyl)-5-(pyrimidin-2-ylamino)-benzamide;
2-Chloro-N-(1-hydroxy-cyclohexylmethyl)-5-(methyl-pyrimidin-2-yl-amino)-benzamide;
2-Chloro-N-(1-hydroxy-cyclohexylmethyl)-5-[(2-methoxy-ethyl)-pyrimidin-2-yl-amino]-benzamide;
2-Chloro-N-(1-hydroxy-cyclohexylmethyl)-5-(4-methylsulfanyl-pyrimidin-2-ylamino)-benzamide;
2-Chloro-N-(1-hydroxy-cyclohexylmethyl)-5-[methyl-(4-methylsulfanyl-pyrimidin-2-yl)-amino]-benzamide;
2-Chloro-N-(1-hydroxy-cyclohexylmethyl)-5-[(4-methoxy-pyrimidin-2-yl)-methyl-amino]-benzamide;
2-Chloro-N-(1-hydroxy-cyclohexylmethyl)-5-[methyl-(6-oxo-1,6-dihydro-pyrimidin-2-yl)-amino]-benzamide;
2-Chloro-N-(1-hydroxy-cyclohexylmethyl)-5-(pyrimidin-5-ylamino)-benzamide;
2-Chloro-N-(1-hydroxy-cyclohexylmethyl)-5-[(2-methoxy-ethyl)-pyrimidin-5-yl-amino]-benzamide;
2-Chloro-N-(1-hydroxy-cyclohexylmethyl)-5-(pyrazin-2-ylamino)-benzamide;
2-Chloro-N-(1-hydroxy-cyclohexylmethyl)-5-(methyl-pyrazin-2-yl-amino)-benzamide;
2-Chloro-N-(1-hydroxy-cyclohexylmethyl)-5-[(2-methoxy-ethyl)-pyrazin-2-yl-amino]-benzamide;
2-Chloro-5-[(6-chloro-pyrazin-2-yl)-methyl-amino]-N-(1-hydroxy-cyclohexylmethyl)-benzamide;
2-Chloro-N-(1-hydroxy-cyclohexylmethyl)-5-[(6-methoxy-pyrazin-2-yl)-methyl-amino]-benzamide;
2-Chloro-N-(1-hydroxy-cyclohexylmethyl)-5-[methyl-(6-methylamino-pyrazin-2-yl)-amino]-benzamide;
2-Chloro-5-[(6-dimethylamino-pyrazin-2-yl)-methyl-amino]-N-(1-hydroxy-cyclohexylmethyl)-benzamide;
2-Chloro-N-(1-hydroxy-cyclohexylmethyl)-5-[methyl-(6-oxo-1,6-dihydro-pyrazin-2-yl)-amino]-benzamide;
2-Chloro-5-[(3-chloro-pyrazin-2-yl)-methyl-amino]-N-(1-hydroxy-cyclohexylmethyl)-benzamide;
2-Chloro-5-[(5-chloro-pyrazin-2-yl)-methyl-amino]-N-(1-hydroxy-cyclohexylmethyl)-benzamide;
2-Chloro-N-(1-hydroxy-cyclohexylmethyl)-5-[(5-methoxy-pyrazin-2-yl)-methyl-amino]-benzamide;
2-Chloro-5-[(6-chloro-pyridazin-3-yl)-methyl-amino]-N-(1-hydroxy-cyclohexylmethyl)-benzamide;
2-Chloro-N-(1-hydroxy-cyclohexylmethyl)-5-[methyl-(6-oxo-1,6-dihydro-pyridazin-3-yl)-amino]-benzamide;
2-Chloro-N-(1-hydroxy-cyclohexylmethyl)-5-[(6-methoxy-pyridazin-3-yl)-methyl-amino]-benzamide;
2-Chloro-N-(1-hydroxy-cyclohexylmethyl)-5-(pyridin-2-ylamino)-benzamide;
2-Chloro-N-(1-hydroxy-cyclohexylmethyl)-5-(methyl-pyridin-2-yl-amino)-benzamide;
2-Chloro-N-(1-hydroxy-cyclohexylmethyl)-5-(6-oxo-1,6-dihydro-pyridin-2-ylamino)-benzamide;
2-Chloro-N-(1-hydroxy-cyclohexylmethyl)-5-[methyl-(6-oxo-1,6-dihydro-pyridin-2-yl)-amino]-benzamide;
2-Chloro-N-(1-hydroxy-cyclohexylmethyl)-5-(1-methyl-6-oxo-1,6-dihydro-pyridin-2-ylamino)-benzamide;
2-Chloro-N-(1-hydroxy-cyclohexylmethyl)-5-[1-(2-methoxy-ethyl)-6-oxo-1,6-dihydro-pyridin-2-ylamino]-benzamide;
2-Chloro-N-(1-hydroxy-cyclohexylmethyl)-5-{[1-(2-methoxy-ethyl)-6-oxo-1,6-dihydro-pyridin-2-yl]-methyl-amino}-benzamide;
2-Chloro-N-(1-hydroxy-cyclohexylmethyl)-5-(1-methyl-2-oxo-1,2-dihydro-pyridin-3-ylamino)-benzamide;
2-Chloro-N-(1-hydroxy-cyclohexylmethyl)-5-(2-methyl-2H-pyrazol-3-ylamino)-benzamide;
2-Chloro-N-(1-hydroxy-cyclohexylmethyl)-5-[methyl-(2-methyl-2H-pyrazol-3-yl)-amino]-benzamide;
2-Chloro-5-(6-fluoro-pyridin-2-yloxy)-N-(1-hydroxy-cyclohexylmethyl)-benzamide;
2-Chloro-5-(4-fluoro-pyridin-2-yloxy)-N-(1-hydroxy-cyclohexylmethyl)-benzamide;
2-Chloro-5-(2-chloro-pyridin-4-yloxy)-N-(1-hydroxy-cyclohexylmethyl)-benzamide;
2-Chloro-N-(1-hydroxy-cyclohexylmethyl)-5-(pyridin-2-yloxy)-benzamide;
2-Chloro-N-(1-hydroxy-cyclohexylmethyl)-5-(pyridin-4-yloxy)-benzamide;
2-Chloro-N-(1-hydroxy-cyclohexylmethyl)-5-(pyridin-3-yloxy)-benzamide;
2-Chloro-5-(6-chloro-pyridin-2-yloxy)-N-(1-hydroxy-cyclohexylmethyl)-benzamide;
2-Chloro-N-(1-hydroxy-cyclohexylmethyl)-5-(6-methylamino-pyridin-2-yloxy)-benzamide;
2-Chloro-5-(6-dimethylamino-pyridin-2-yloxy)-N-(1-hydroxy-cyclohexylmethyl)-benzamide;
2-Chloro-N-(1-hydroxy-cyclohexylmethyl)-5-(6-methoxy-pyridin-2-yloxy)-benzamide;
2-Chloro-N-(1-hydroxy-cyclohexylmethyl)-5-(6-oxo-1,6-dihydro-pyridin-2-yloxy)-benzamide;
2-Chloro-N-(1-hydroxy-cyclohexylmethyl)-5-(1-methyl-6-oxo-1,6-dihydro-pyridin-2-yloxy)-benzamide;
2-Chloro-N-(1-hydroxy-cyclohexylmethyl)-5-(pyrimidin-4-yloxy)-benzamide;
2-Chloro-5-(2-chloro-pyrimidin-4-yloxy)-N-(1-hydroxy-cyclohexylmethyl)-benzamide;
2-Chloro-N-(1-hydroxy-cyclohexylmethyl)-5-(2-methylamino-pyrimidin-4-yloxy)-benzamide;
2-Chloro-5-(2-dimethylamino-pyrimidin-4-yloxy)-N-(1-hydroxy-cyclohexylmethyl)-benzamide;
2-Chloro-N-(4,4-difluoro-1-hydroxy-cyclohexylmethyl)-5-(2-oxo-1,2-dihydro-pyrimidin-4-yloxy)-benzamide;
2-Chloro-N-(1-hydroxy-cyclohexylmethyl)-5-(2-oxo-2,3-dihydro-pyrimidin-4-yloxy)-benzamide;
2-Chloro-N-(1-hydroxy-cyclohexylmethyl)-5-(2-methoxy-pyrimidin-4-yloxy)-benzamide;
2-Chloro-N-(1-hydroxy-cyclohexylmethyl)-5-(pyrazin-2-yloxy)-benzamide;
2-Chloro-5-(6-chloro-pyrazin-2-yloxy)-N-(1-hydroxy-cyclohexylmethyl)-benzamide;
2-Chloro-N-(1-hydroxy-cyclohexylmethyl)-5-(6-methoxy-pyrazin-2-yloxy)-benzamide;
2-Chloro-N-(1-hydroxy-cyclohexylmethyl)-5-(6-methylamino-pyrazin-2-yloxy)-benzamide;
2-Chloro-5-(6-dimethylamino-pyrazin-2-yloxy)-N-(1-hydroxy-cyclohexylmethyl)-benzamide;
2-Chloro-N-(1-hydroxy-cyclohexylmethyl)-5-(6-oxo-1,6-dihydro-pyrazin-2-yloxy)-benzamide;
2-Chloro-5-(6-chloro-pyridazin-3-yloxy)-N-(1-hydroxy-cyclohexylmethyl)-benzamide;
2-Chloro-N-(1-hydroxy-cyclohexylmethyl)-5-(6-methoxy-pyridazin-3-yloxy)-benzamide;
2-Chloro-N-(1-hydroxy-cyclohexylmethyl)-5-(6-oxo-1,6-dihydro-pyridazin-3-yloxy)-benzamide;
2-Chloro-N-(1-hydroxy-cyclohexylmethyl)-5-(pyridazin-3-yloxy)-benzamide;
2-Chloro-N-(1-hydroxy-cyclohexylmethyl)-5-(5-methoxy-pyridazin-3-yloxy)-benzamide;
2-Chloro-N-(1-hydroxy-cyclohexylmethyl)-5-(pyridin-2-ylsulfanyl)-benzamide;
2-Chloro-N-(1-hydroxy-cyclohexylmethyl)-5-(pyridine-2-sulfinyl)-benzamide;
2-Chloro-N-(1-hydroxy-cyclohexylmethyl)-5-(pyridine-2-sulfonyl)-benzamide;
2-Chloro-5-(2-chloro-pyrimidin-4-ylsulfanyl)-N-(1-hydroxy-cyclohexylmethyl)-benzamide;
2-Chloro-5-(2-chloro-pyrimidine-4-sulfinyl)-N-(1-hydroxy-cyclohexylmethyl)-benzamide;
2-Chloro-N-(1-hydroxy-cyclohexylmethyl)-5-(2-oxo-1,2-dihydro-pyrimidin-4-ylsulfanyl)-benzamide;
2-Chloro-N-(4,4-difluoro-1-hydroxy-cyclohexylmethyl)-5-(2-oxo-1,2-dihydro-pyrimidin-4-ylsulfanyl)-benzamide;
2-Chloro-N-(1-hydroxy-cyclohexylmethyl)-5-(2-methoxy-pyrimidin-4-ylsulfanyl)-benzamide;
2-Chloro-N-(1-hydroxy-cyclohexylmethyl)-5-(2-methoxy-pyrimidine-4-sulfinyl)-benzamide;
2-Chloro-N-(1-hydroxy-cyclohexylmethyl)-5-(2-methoxy-pyrimidine-4-sulfonyl)-benzamide;
2-Chloro-N-(1-hydroxy-cyclohexylmethyl)-5-(2-oxo-2H-pyridin-1-ylmethyl)-benzamide;
2-Chloro-N-(1-hydroxy-cyclohexylmethyl)-5-(2-oxo-2H-pyrimidin-1-ylmethyl)-benzamide;
2-Chloro-5-(2,4-dioxo-3,4-dihydro-2H-pyrimidin-1-ylmethyl)-N-(1-hydroxy-cyclohexylmethyl)-benzamide;
2-Chloro-N-(1-hydroxy-cyclohexylmethyl)-5-pyridin-2-ylmethyl-benzamide;
2-Chloro-N-(1-hydroxy-cyclohexylmethyl)-5-(2-methylsulfanyl-pyrimidin-4-ylmethyl)-benzamide;
2-Chloro-N-(1-hydroxy-cyclohexylmethyl)-5-(2-oxo-1,2-dihydro-pyrimidin-4-ylmethyl)-benzamide;
2-Chloro-N-(1-hydroxy-cyclohexylmethyl)-5-(2-methoxy-pyrimidin-4-ylmethyl)-benzamide;
2-Chloro-N-(4,4-difluoro-1-hydroxy-cyclohexyl methyl)-5-(2-oxo-1,2-di hydro-pyrimidin-4-ylmethyl)-benzamide;
2-Chloro-N-(1-hydroxy-cyclohexylmethyl)-5-(2-oxo-1,2-dihydro-pyrimidine-4-carbonyl)-benzamide;
2-Chloro-5-[difluoro-(2-oxo-1,2-dihydro-pyrimidin-4-yl)-methyl]-N-(1-hydroxy-cyclohexylmethyl)-benzamide;
2-Chloro-5[fluoro-(2-oxo-1,2-dihydro-pyrimidin-4-yl)-methyl]-N-(1-hydroxy-cyclohexylmethyl)-benzamide;
5-[(2-Chloro-pyrimidin-4-yl)-methyl-amino]-N-(1-hydroxy-cyclohexylmethyl)-2-methyl-benzamide;
N-(1-Hydroxy-cyclohexylmethyl)-5-[(2-methoxy-pyrimidin-4-yl)-methyl-amino]-2-methyl-benzamide; and
N-(1-Hydroxy-cyclohexylmethyl)-2-methyl-5-[methyl-(2-oxo-1,2-dihydro-pyrimidin-4-yl)-amino]-benzamide;
   or salts (in particular pharmaceutically acceptable salts) of such compounds;
   it is to be understood for any of the above listed compounds, that a stereogenic center, which is not specifically assigned, may be in absolute (R)— or absolute (S)-configuration.
   55) Further preferred compounds of formula (I) as defined in embodiment 1) are selected from the group consisting of:
2-Chloro-N-(1-hydroxy-cyclohexylmethyl)-5-[1-hydroxy-1-(2-oxo-1,2-dihydro-pyrimidin-4-yl)-ethyl]-benzamide;
2-Chloro-N-(1-hydroxy-cyclohexylmethyl)-5-[(1S)-1-hydroxy-1-(2-oxo-1,2-dihydro-pyrimidin-4-yl)-ethyl]-benzamide;
2-Chloro-N-(1-hydroxy-cyclohexylmethyl)-5-[(1R)-1-hydroxy-1-(2-oxo-1,2-dihydro-pyrimidin-4-yl)-ethyl]-benzamide; and
2-Chloro-N-(1-hydroxy-cyclohexylmethyl)-5-[1-hydroxy-1-(2-methoxy-pyrimidin-4-yl)-ethyl]-benzamide;
   or salts (in particular pharmaceutically acceptable salts) of such compounds;
   it is to be understood for any of the above listed compounds, that a stereogenic center, which is not specifically assigned, may be in absolute (R)— or absolute (S)-configuration.

The present invention also includes isotopically labelled, especially ²H (deuterium) labelled compounds of formula (I), which compounds are identical to the compounds of formula (I) except that one or more atoms have each been replaced by an atom having the same atomic number but an atomic mass different from the atomic mass usually found in nature. Isotopically labelled, especially ²H (deuterium) labelled compounds of formula (I) and salts thereof are within the scope of the present invention. Substitution of hydrogen with the heavier isotope ²H (deuterium) may lead to greater metabolic stability, resulting e.g. in increased in-vivo half-life or reduced dosage requirements, or may lead to reduced inhibition of cytochrome P450 enzymes, resulting e.g. in an improved safety profile. In one embodiment of the invention, the compounds of formula (I) are not isotopically labelled, or they are labelled only with one or more deuterium atoms. In a sub-embodiment, the compounds of formula (I) are not isotopically labelled at all. Isotopically labelled compounds of formula (I) may be prepared in analogy to the methods described hereinafter, but using the appropriate isotopic variation of suitable reagents or starting materials.

The term “pharmaceutically acceptable salts” refers to non-toxic, inorganic or organic acid and/or base addition salts, Lit. e.g. “Salt selection for basic drugs”, Int. J. Pharm. (1986), 33, 201-217.

Where the plural form is used for compounds, salts, pharmaceutical compositions, diseases and the like, this is intended to mean also a single compound, salt, or the like.

The compounds of formula (I) according to any one of embodiments 1) to 55), or pharmaceutically acceptable salts thereof, are suitable for use as medicaments. In particular, compounds of formula (I) modulate the P2X₇ receptor, i.e. they act as P2X₇ receptor antagonists, and are useful for the prevention or treatment of diseases which are associated with the activation of the P2X₇ receptor such as pain; neurodegenerative and neuroinflammatory diseases; bone and joint diseases; obstructive diseases of the airways; cardiovascular diseases; eye diseases; skin diseases; abdominal and gastrointestinal tract diseases; genitourinary diseases; cancer; other auto-immune and allergic disorders; and other disorders with an inflammatory or immunological component.

In particular, the compounds of formula (I) according to any one of embodiments 1) to 55), or pharmaceutically acceptable salts thereof, are suitable for the prevention or treatment of pain. Pain refers to acute pain; chronic pain; pain associated with sprains and strains; chronic articular pain; pain associated with rheumatic fever; musculoskeletal pain; lower back and neck pain; inflammatory pain; neuropathic pain; visceral pain; pain associated with influenza or other viral infections; pain associated with cancer and tumor invasion; joint and bone pain; atypical facial pain; pain associated with migraine, toothache and dysmenorrhea; headache including tension headache and cluster headaches; pain associated with myocardial ischemia; pain associated with functional bowel disorders; sympathetically maintained pain; myositis; pain associated with cancer chemotherapy; and post operative pain.

Neuropathic pain includes especially diabetic neuropathy, sciatica, non-specific lower back pain, trigeminal neuralgia, multiple sclerosis pain, fibromyalgia, HIV-related neuropathy, post-herpetic neuralgia, trigeminal neuralgia, and pain resulting from physical trauma, amputation, phantom limb syndrome, spinal surgery, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Chronic articular pain conditions include especially rheumatoid arthritis, osteoarthritis, rheumatoid spondylitis, gouty arthritis and juvenile arthritis.

Pain associated with functional bowel disorders includes especially non-ulcer dyspepsia, non-cardiac chest pain and irritable bowel syndrome.

Further, the compounds of formula (I) according to any one of embodiments 1) to 55), or pharmaceutically acceptable salts thereof, are suitable for the prevention or treatment of neurodegenerative and neuroinflammatory diseases. Neurodegenerative and neuro-inflammatory diseases include Alzheimer's disease and other dementing disorders including, but not limited to, Creutzfeldt-Jakob disease (CJD) and new variant Creutzfeldt-Jakob disease (nvCJD); Amyotrophic lateral sclerosis, amyloidosis; multiple sclerosis and other demyelinating syndromes; cerebral atherosclerosis and vasculitis; temporal arteritis; myasthenia gravis; Huntington's disease; Lewy Body dementia; and Parkinson's disease.

Further, the compounds of formula (I) according to any one of embodiments 1) to 55), or pharmaceutically acceptable salts thereof, are suitable for the prevention or treatment of bone and joint diseases. Bone and joint diseases include arthritides such as rheumatoid arthritis, osteoarthritis, gout or crystal arthropathy; intervertebral disc degeneration; temporomandibular joint degeneration; bone remodelling disease such as osteoporosis, Paget's disease or osteonecrosis; polychondritis; scleroderma; mixed connective tissue disorder; spondyloarthropathies; periodontal disease such as periodontitis; arthritides associated with or including osteoarthritis/osteoarthrosis, both primary and secondary to, for example, congenital hip dysplasia; cervical and lumbar spondylitis; Still's disease; seronegative spondyloarthropathies including ankylosing spondylitis, psoriatic arthritis, reactive arthritis and undifferentiated spondyloarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; Familial Mediterranean fever, Muckle-Wells syndrome, and Familial Hibernian Fever, Kikuchi disease; and drug-induced arthalgias, tendonitis, and myopathies including dystrophies and other inflammatory myopathies.

Further, the compounds of formula (I) according to any one of embodiments 1) to 55), or pharmaceutically acceptable salts thereof, are suitable for the prevention or treatment of obstructive diseases of the airways. Obstructive diseases of the airways include asthma, including bronchial, allergic, intrinsic, and extrinsic asthma, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; and acute viral infection including the common cold, and infection due to respiratory syncytial virus, influenza, coronavirus (including SARS) and adenovirus.

Further, the compounds of formula (I) according to any one of embodiments 1) to 55), or pharmaceutically acceptable salts thereof, are suitable for the prevention or treatment of cardiovascular diseases. Cardiovascular diseases include atherosclerosis, affecting the coronary and peripheral circulation; pericarditis; myocarditis; inflammatory and auto-immune cardiomyopathies including myocardial sarcoid; ischaemic reperfusion injuries; endocarditis, valvulitis, and aortitis including infective (for example syphilitic); vasculitides; and disorders of the proximal and peripheral veins including phlebitis and thrombosis, including deep vein thrombosis and complications of varicose veins.

Further, the compounds of formula (I) according to any one of embodiments 1) to 55), or pharmaceutically acceptable salts thereof, are suitable for the prevention or treatment of eye diseases. Eye diseases include blepharitis; conjunctivitis, including perennial and vernal allergic conjunctivitis; iritis; anterior and posterior uveitis; choroiditis; autoimmune, degenerative or inflammatory disorders affecting the retina; ophthalmitis including sympathetic ophthalmitis; sarcoidosis; and infections of the eyes including viral, fungal, and bacterial infections.

Further, the compounds of formula (I) according to any one of embodiments 1) to 55), or pharmaceutically acceptable salts thereof, are suitable for the prevention or treatment of skin diseases. Skin diseases include psoriasis, skin burn, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; and drug-induced disorders including fixed drug eruptions.

Further, the compounds of formula (I) according to any one of embodiments 1) to 55), or pharmaceutically acceptable salts thereof, are suitable for the prevention or treatment of abdominal and gastrointestinal tract diseases. Abdominal and gastrointestinal tract diseases include hepatitis, including autoimmune, alcoholic and viral hepatitis; fibrosis and cirrhosis of the liver; cholecystitis; pancreatitis, both acute and chronic; non-inflammatory diarrhea; glossitis, gingivitis, periodontitis; oesophagitis, including reflux; eosinophilic gastro-enteritis, mastocytosis, Crohn's disease, colitis including ulcerative colitis, proctitis, pruritis ani; Coeliac disease, irritable bowel disease/syndrome, and food-related allergies which may have effects remote from the gut, for example migraine, rhinitis or eczema; allograft rejection including acute and chronic allograft rejection following, for example, transplantation of kidney, heart, liver, lung, bone marrow, skin or cornea or following blood transfusion; and chronic graft versus host disease;

Further, the compounds of formula (I) according to any one of embodiments 1) to 55), or pharmaceutically acceptable salts thereof, are suitable for the prevention or treatment of genitourinary diseases. Genitourinary diseases include nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvovaginitis; Peyronie's disease; and erectile dysfunction, both male and female.

Further, the compounds of formula (I) according to any one of embodiments 1) to 55), or pharmaceutically acceptable salts thereof, are suitable for the prevention or treatment of cancer. The treatment of cancer includes the treatment of brain tumors, prostate, lung, breast, ovarian, bowel and colon, stomach, pancreatic, skin and bone marrow (including leukaemias) and lymphoproliferative systems, such as non-Hodgkin's and Hodgkin's lymphoma; including the prevention and treatment of metastatic disease and tumor recurrences, and paraneoplastic syndromes.

Further, the compounds of formula (I) according to any one of embodiments 1) to 55), or pharmaceutically acceptable salts thereof, are suitable for the prevention or treatment of other auto-immune and allergic disorders. Other auto-immune and allergic disorders include Hashimoto's thyroiditis, Graves' disease, Addison's disease, diabetes mellitus, idiopathic thrombocytopaenic purpura, eosinophilic fasciitis, hyper-IgE syndrome, and antiphospholipid syndrome.

Further, the compounds of formula (I) according to any one of embodiments 1) to 55), or pharmaceutically acceptable salts thereof, are suitable for the prevention or treatment of other disorders with an inflammatory or immunological component. Other disorders with an inflammatory or immunological component include acquired immune deficiency syndrome (AIDS), leprosy, Sezary syndrome, and paraneoplastic syndromes.

Further, the compounds of formula (I) according to any one of embodiments 1) to 55), or pharmaceutically acceptable salts thereof, are suitable for the prevention or treatment of mood, sleep and anxiety disorders.

Further, the compounds of formula (I) according to any one of embodiments 1) to 55), or pharmaceutically acceptable salts thereof, are suitable for the prevention or treatment of injury induced trauma and spinal cord injury.

Especially, compounds of formula (I) according to any one of embodiments 1) to 55), or pharmaceutically acceptable salts thereof, are suitable for the prevention or treatment of diseases selected from one, several or all of the following groups of diseases and disorders:
1) Pain, wherein pain refers to acute pain; chronic pain; pain associated with sprains and strains; chronic articular pain; pain associated with rheumatic fever; musculoskeletal pain; lower back and neck pain; inflammatory pain; neuropathic pain; visceral pain; pain associated with influenza or other viral infections; pain associated with cancer and tumor invasion; joint and bone pain; atypical facial pain; pain associated with migraine, toothache and dysmenorrhea; headache including tension headache and cluster headaches; pain associated with myocardial ischemia; pain associated with functional bowel disorders; sympathetically maintained pain; myositis; pain associated with cancer chemotherapy; and post operative pain;
Neuropathic pain includes especially diabetic neuropathy, sciatica, non-specific lower back pain, trigeminal neuralgia, multiple sclerosis pain, fibromyalgia, HIV-related neuropathy, post-herpetic neuralgia, trigeminal neuralgia, and pain resulting from physical trauma, amputation, phantom limb syndrome, spinal surgery, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia);
Chronic articular pain conditions include especially rheumatoid arthritis, osteoarthritis, rheumatoid spondylitis, gouty arthritis and juvenile arthritis; Pain associated with functional bowel disorders includes especially non-ulcer dyspepsia, non-cardiac chest pain and irritable bowel syndrome;
2) Neurodegenerative and neuro-inflammatory diseases such as Alzheimer's disease and other dementing disorders including, but not limited to, Creutzfeldt-Jakob disease (CJD) and new variant Creutzfeldt-Jakob disease (nvCJD); amyloidosis; Amyotrophic lateral sclerosis, multiple sclerosis and other demyelinating syndromes; cerebral atherosclerosis and vasculitis; temporal arteritis; myasthenia gravis; Huntington's disease; Lewy Body dementia; and Parkinson's disease;
3) Bone and joint diseases such as arthritides such as rheumatoid arthritis, osteoarthritis, gout or crystal arthropathy; intervertebral disc degeneration; temporomandibular joint degeneration; bone remodelling disease such as osteoporosis, Paget's disease or osteonecrosis; polychondritis; scleroderma; mixed connective tissue disorder; spondyloarthropathies; periodontal disease such as periodontitis; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; Muckle-Wells syndrome, and Familial Hibernian Fever, Kikuchi disease; and drug-induced arthalgias, tendonitis, and myopathies;
4) Obstructive diseases of the airways such as chronic obstructive pulmonary disease (COPD); cystic fibrosis; lung emphysema; sarcoidosis; farmer's lung and related diseases; lung fibrosis, including fibrosis complicating tuberculosis; and chronic cough associated with inflammatory and secretory conditions of the airways;
5) Cardiovascular diseases such as inflammatory and auto-immune cardio-myopathies;
6) Eye diseases such as degenerative or inflammatory disorders affecting the retina;
7) Skin diseases such as psoriasis, skin burn, atopic dermatitis, contact dermatitis or other eczematous dermatoses; and discoid lupus erythematosus;
8) Abdominal and gastrointestinal tract diseases such as fibrosis and cirrhosis of the liver; cholecystitis; pancreatitis, both acute and chronic; Crohn's disease; colitis including ulcerative colitis; and irritable bowel disease/syndrome;
9) Genitourinary diseases such as nephritis including interstitial and glomerulonephritis; nephrotic syndrome; and cystitis including acute and chronic (interstitial) cystitis; and
10) Other auto-immune and allergic disorders such as Hashimoto's thyroiditis, Graves' disease, Addison's disease, diabetes mellitus, idiopathic thrombocytopaenic purpura, eosinophilic fasciitis, hyper-IgE syndrome, and antiphospholipid syndrome.

Most preferably, compounds of formula (I) according to any one of embodiments 1) to 55), or pharmaceutically acceptable salts thereof, are suitable for the prevention or treatment of diseases selected from one, several or all of the following groups of diseases and disorders:
1) Pain, wherein pain refers to acute pain; chronic pain; pain associated with sprains and strains; chronic articular pain; pain associated with rheumatic fever; musculoskeletal pain (preferred); lower back and neck pain; inflammatory pain; neuropathic pain (preferred); visceral pain; pain associated with influenza or other viral infections; pain associated with cancer and tumor invasion; joint and bone pain; atypical facial pain; pain associated with migraine, toothache and dysmenorrhea; headache including tension headache and cluster headaches; pain associated with myocardial ischemia; pain associated with functional bowel disorders; sympathetically maintained pain; myositis; pain associated with cancer chemotherapy; and post operative pain;
   Neuropathic pain includes especially diabetic neuropathy, sciatica, non-specific lower back pain, trigeminal neuralgia, multiple sclerosis pain, fibromyalgia, HIV-related neuropathy, post-herpetic neuralgia, trigeminal neuralgia, and pain resulting from physical trauma, amputation, phantom limb syndrome, spinal surgery, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia);
   Chronic articular pain conditions include especially rheumatoid arthritis, osteoarthritis, rheumatoid spondylitis, gouty arthritis and juvenile arthritis;
   Pain associated with functional bowel disorders includes especially non-ulcer dyspepsia, non-cardiac chest pain and irritable bowel syndrome;
2) Rheumatoid arthritis and osteoarthritis;
3) Chronic obstructive pulmonary disease (COPD); and
4) Crohn's disease.

The invention also relates to the use of a compound of formula (I) according to any one of embodiments 1) to 55) for the preparation of pharmaceutical compositions for the treatment and/or prophylaxis of the above-mentioned diseases.

The present invention also relates to pharmaceutically acceptable salts and to pharmaceutical compositions and formulations of compounds of formula (I) according to any one of embodiments 1) to 55).

A pharmaceutical composition according to the present invention contains at least one compound of formula (I) according to any one of embodiments 1) to 55) (or a pharmaceutically acceptable salt thereof) as the active agent and optionally carriers and/or diluents and/or adjuvants.

The compounds of formula (I) according to any one of embodiments 1) to 55) and their pharmaceutically acceptable salts can be used as medicaments, e.g. in the form of pharmaceutical compositions for enteral (such especially oral) or parenteral administration (including topical application or inhalation).

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula (I) or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

The present invention also relates to a method for the prevention or treatment of a disease or disorder mentioned herein comprising administering to a subject a pharmaceutically active amount of a compound of formula (I) according to any one of embodiments 1) to 55), or a pharmaceutically acceptable salt thereof.

Any reference to a compound of formula (I) in this text is to be understood as referring also to the salts (and especially the pharmaceutically acceptable salts) of such compounds, as appropriate and expedient. The preferences indicated for the compounds of formula (I) of course apply mutatis mutandis to the salts and pharmaceutically acceptable salts of the compounds of formula (I). The same applies to these compounds as medicaments, to pharmaceutical compositions containing these compounds as active principles or to the uses of these compounds for the manufacture of a medicament for the treatment of the diseases according to this invention.

Unless used regarding temperatures, the term “about” (or alternatively “around”) placed before a numerical value “X” refers in the current application to an interval extending from X minus 10% of X to X plus 10% of X, and preferably to an interval extending from X minus 5% of X to X plus 5% of X. In the particular case of temperatures, the term “about” (or alternatively “around”) placed before a temperature “Y” refers in the current application to an interval extending from the temperature Y minus 10° C. to Y plus 10° C., and preferably to an interval extending from Y minus 5° C. to Y plus 5° C. Besides, the term “room temperature” (rt) as used herein refers to a temperature of about 25° C. Whenever the word “between” is used to describe a numerical range, it is to be understood that the end points of the indicated range are explicitly included in the range. For example: if a temperature range is described to be between 40° C. and 80° C., this means that the end points 40° C. and 80° C. are included in the range; or if a variable is defined as being an integer between 1 and 4, this means that the variable is the integer 1, 2, 3, or 4.

The compounds of Formula (I) can be manufactured by the methods given below, by the methods given in the Examples or by analogous methods. Optimum reaction conditions may vary with the particular reactants or solvents used, but such conditions can be determined by a person skilled in the art by routine optimisation procedures.

If not indicated otherwise, the generic groups R¹, R², R³, R⁴, R⁵, R⁶, R⁷, R⁸, R⁹, n and Y are as defined for formula (I). Other abbreviations used are defined in the experimental section.

In some instances the generic groups R¹, R², R³, R⁴, R⁵, R⁶, R⁷, R⁸, R⁹, n and Y might be incompatible with the assembly illustrated in the schemes below and will therefore require the use of protecting groups (PG). The use of protecting groups is well known in the art (see for example “Protective Groups in Organic Synthesis”, T. W. Greene, P. G. M. Wuts, Wiley-Interscience, 1999). For the purposes of this discussion, it will be assumed that such protecting groups are as necessary in place.

### A. Synthesis of Final Products

Compounds of formula Ia and Ib wherein Y represents NR⁹ can be prepared following the procedures outlined in Scheme 1 below.

The compounds of formula V can be prepared (Scheme 1) by a Buchwald-Hartwig type of reaction, using a commercially available iodide of formula VII (or a commercially available aniline of formula VIII, respectively) and an aniline of formula R¹—NH₂ (or a halide, preferably an iodide of formula R¹—X, respectively), in the presence of a suitable palladium catalyst such as palladium(II) acetate or tris(dibenzylideneacetone) dipalladium, in the presence of a suitable ligand such as 2,2′-bis(diphenylphosphino)-1,1′-binaphthalene or 4,5-bis(diphenylphosphino)-9,9-dimethylxanthene, in the presence of a suitable base such as Cs₂CO₃ or sodium phenoxide and heating in a suitable solvent such as dioxane at a temperature between 90° C. and 120° C.

The compounds of formula VI can be prepared (Scheme 1) by alkylation of a compound of formula V using an appropriate alkyl iodide or bromide of formula R⁹—X, in the presence of a suitable base such as Cs₂CO₃ or potassium carbonate and in the presence of a suitable organic solvent such as DMF or THF, preferably at a temperature between RT and 60° C.

The compounds of formula III (or IV, respectively) can be prepared (Scheme 1) by hydrolysis of a compound of formula V (or VI, respectively) using standard conditions such as NaOH or LiOH in a mixture of water and a suitable organic solvent such as THF, MeOH or EtOH, preferably at a temperature between RT and 45° C.

The compounds of formula Ia (or Ib, respectively) can be prepared (Scheme 1) by coupling an acid of formula III (or IV, respectively) with an amine of formula II using standard amide coupling reagents such as TBTU, EDC.HCl/HOBT, HATU, PyBOP or PyCloP in the presence of a suitable base such as NEt₃ or DIPEA and in a suitable solvent such as DCM, THF or DMF, preferably at a temperature between RT and 45° C.

Alternatively, the compounds of formula Ib can be prepared (Scheme 1) by alkylation of a compound of formula Ia with an appropriate alkyl iodide or bromide of formula R⁹—X using standard conditions such as a suitable base (preferably Cs₂CO₃) and a suitable organic solvent such as DMF or THF, preferably at a temperature between RT and 45° C.

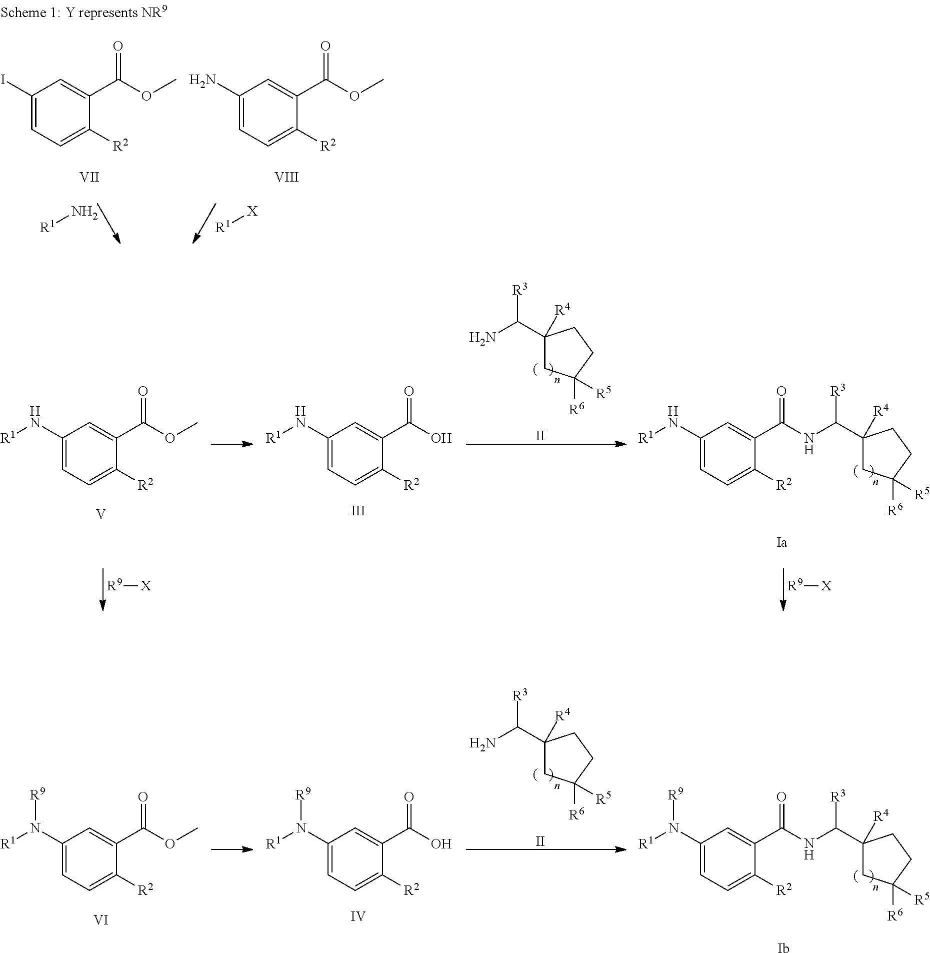

Compounds of formula Ic, wherein Y represents O or S, Id and le can be prepared following the procedures outlined in Scheme 2 below.

The compounds of formula IX can be prepared (Scheme 2) by an aromatic nucleophilic substitution of a commercially available phenol or thiophenol, respectively of formula XI with a halide (preferably a bromide or iodide) of formula R¹—X, in the presence of a suitable base such as Cs₂CO₃ and heating in a suitable sovent such as DMSO at a temperature between 60° C. and 110° C.

The compounds of formula Ic can be prepared (Scheme 2) by coupling an acid of formula IX with an amine of formula II using standard amide coupling conditions such as those already described for the synthesis of the compounds of formula Ia and Ib (Scheme 1).

The compounds of formula Id can be prepared (Scheme 2) by oxidation of a compound of formula Ic, wherein Y represents S, using a suitable oxidating reagent such as 3-chloroperbenzoic acid in a suitable solvent such as DCM at a temperature around 0° C.

The compounds of formula Ie can be prepared (Scheme 2) by oxidation of a compound of formula Ic, wherein Y represents S, using a suitable oxidating reagent such as 3-chloroperbenzoic acid in a suitable solvent such as DCM at a temperature around RT.

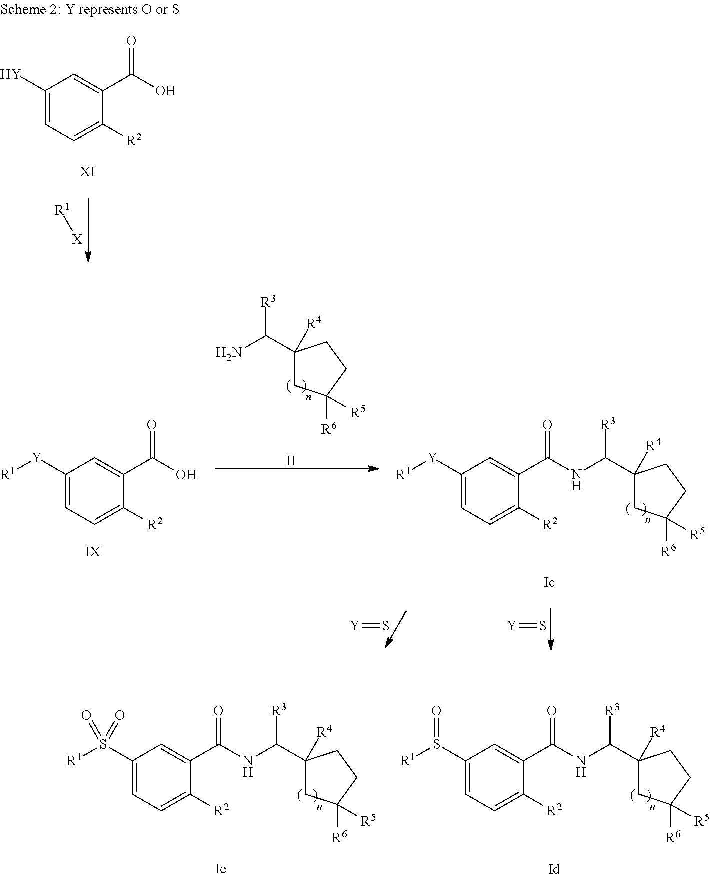

Compounds of formula If and Ig, wherein Y represents C(R⁷R⁸), can be prepared following the procedures outlined in Scheme 3 below.

The compounds of formula XX can be prepared (Scheme 3) by bromination of a commercially available compound of formula XXI, wherein R² represents chloro, using a suitable brominating reagent such as N-bromosuccinimide in the presence of a radical initiator such as 2,2′-azobis(2-methylpropionitrile) and heating in a suitable solvent such as chlorobenzene at a temperature between 90 and 120° C.

The compounds of formula XVIII can be prepared (Scheme 3) by coupling an acid of formula XX with an amine of formula II using standard amide coupling conditions such as EDC.HCl/HOBT, PyBOP in the presence of a suitable base such as DIPEA and in a suitable solvent such as DCM, preferably at a temperature between RT and 45° C. In such conditions, the consecutive substitution of the bromide atom in compound of formula XX with the 1-oxy-benzotriazole group (—OBt) from HOBT is observed.

The compounds of formula If can be prepared (Scheme 3) by nucleophilic substitution of a heterocycle of formula XIX (wherein the letter codes a, b, c and d are selected as follows a=b=c=d=CH; or a=N and b=c=d=CH; or a=N, b=COH and c=d=CH) with a 1-oxy-benzotriazole activated compound of formula XVIII, in the presence of a suitable base such as potassium carbonate or Cs₂CO₃ and heating in a suitable solvent such as DMF or 1,2-dimethoxyethane at a temperature around 90° C.

The compounds of formula XVI can be prepared (Scheme 3) by esterification of compounds of formula XX using standard conditions such as heating in a suitable solvent such as MeOH in the presence of a suitable acid such as sulphuric acid at a temperature around 70° C.

The compounds of formula XVII, wherein Z represents —CH₂CN, can be prepared (Scheme 3) by nucleophilic substitution of a compound of formula XVI with a cyanide precursor such as trimethylsilylcyanide in the presence of a suitable base such as potassium carbonate or Cs₂CO₃ and heating in a suitable solvent such as CH₃CN at a temperature between RT and 60° C.

The compounds of formula XVII, wherein Z represents —CHO, can be prepared (Scheme 3) by an oxidative cleavage of a compound of formula XVI with an oxidative reagent such as 4-methylmorpholine-N-oxide and heating in a suitable solvent such as dioxane at a temperature around 100° C.

The compounds of formula XIII can be prepared (Scheme 3) by a Negishi type reaction.

The compound of formula XVI can be transformed into an organozinc reagent in the presence of preactivated zinc dust in a suitable solvent such as THF. The cross coupling reaction proceeds by the reaction of the organozinc reagent with a halide of formula R¹—X, in the presence of a suitable palladium catalyst such as tetrakis(triphenylphosphine)palladium (0) or tris(dibenzylideneacetone)dipalladium, in the optional presence of a suitable ligand such as tri-2-furylphosphine and in a suitable solvent such as THF at a temperature around RT.

The compounds of formula XIV can be prepared (Scheme 3) by deprotonation of a cyanomethyl derivative of formula XVII, wherein Z represents —CH₂CN, at the α-carbon atom with a base such as NaH in a suitable solvent such as DMF at a temperature around RT and subsequent reaction of the obtained anion with a halide of formula R¹—X wherein R¹ is a 2-chloro-pyrimidin-4-yl or 2-(2-(trimethylsilyl)ethoxy)-pyrimidin-4-yl group. The consecutive oxidative cleavage of the carbon-cyanide bond proceeds in the presence of atmospheric air and additional amounts of a suitable base such as NaH in a suitable solvent such as DMF at a temperature around RT.

Alternatively, the compounds of formula XIV can be prepared (Scheme 3) by an aroylation of a halide of formula R¹—X wherein R¹ is a 2-methoxy-pyrimidin-4-yl group by an aromatic adehyde of formula XVII, wherein Z represents —CHO, in the presence of an azolium salt such as 1,3-dimethylimidazolium iodide and a base such as NaH in a suitable solvent such as dioxane at a temperature around 100° C.

The compounds of formula XV, wherein R⁷ and/or R⁸ represent fluoro, can be prepared (Scheme 3) by the fluorination of a ketone of formula XIV, in the presence of a fluorinating reagent such as bis(2-methoxyethyl)aminosulfur trifluoride at a temperature around 90° C.

Alternatively, the compounds of formula XV, wherein one of R⁷ or R⁸ represents hydrogen and the other represents fluoro, can be prepared (Scheme 3) by a two-step procedure. The ketone of formula XIV can be reduced using standard conditions such as NaBH₄ in the presence of a suitable solvent such as MeOH at a temperature around RT. The resulting alcohol can be fluorinated using a fluorinating reagent such as bis(2-methoxyethyl)aminosulfur trifluoride in the presence of a suitable solvent such as DCM at a temperature around RT.

Alternatively, the compounds of formula XV, wherein one of R⁷ or R⁸ represents (C₁-C₄)alkyl and the other represents hydroxy, can be prepared (Scheme 3) by the addition of a Grignard reagent of formula R⁷—MgX or R⁸—MgX (wherein X represents a bromine or a chlorine atom) to the ketone of formula XIV in the presence of a suitable solvent such as THF or Et₂O at a temperature between −15° C. and RT.

The compounds of formula XII can be prepared (Scheme 3) by hydrolysis of a compound of formula XIII, XIV or XV using standard conditions such as those already described for the synthesis of the compounds of formula III and IV (Scheme 1).

The compounds of formula Ig can be prepared (Scheme 3) by coupling an acid of formula XII with an amine of formula II using standard amide coupling conditions such as those already described for the synthesis of the compounds of formula Ia and Ib (Scheme 1).

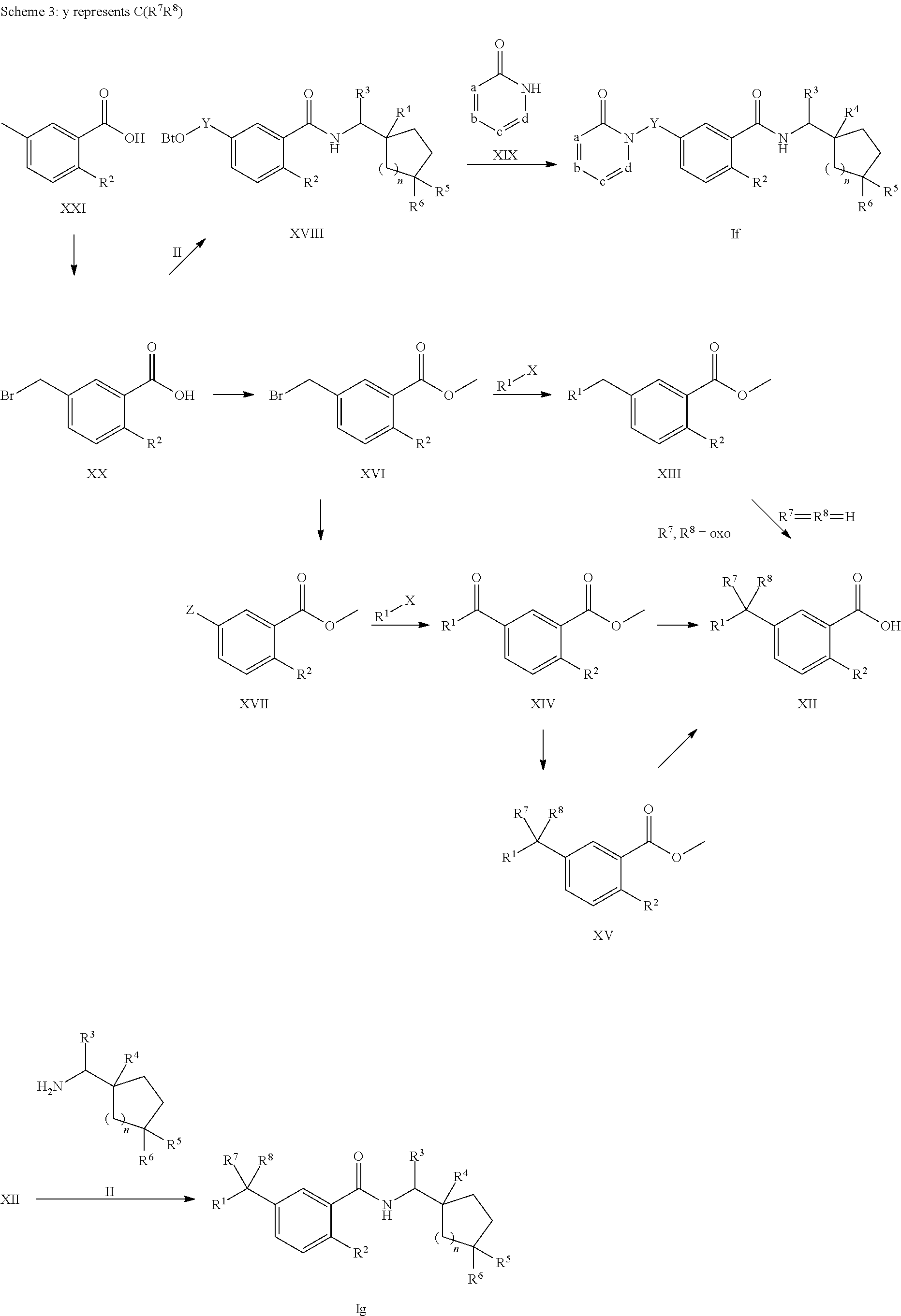

Alternatively, compounds of formula Ii, Ij, Ik, Im, In and Io can be prepared following the procedures outlined in Scheme 4 below. In all the structures of the scheme, one of the letter code, a, b, c or d represents a carbon atom, which is attached to the rest of the molecule via the substituent Y, and the other remaining letter codes are selected from N, CH and CR¹⁰, wherein R¹⁰ represents chloro, to form a 6-membered heteroaryl or a heterocyclyl group R¹ as defined above.

The compounds of formula Ih and Ii, wherein X represents chloro, can be prepared (Scheme 4) as previously described in Schemes 1 (compounds of formula Ia and Ib), 2 (compounds of formula Ic) and 3 (compounds of formula Ig).

The compounds of formula Ii wherein X represents SO₂CH₃ can be prepared (Scheme 4) by oxidation of a compound of formula Ih in the presence of a suitable oxidating reagent such as 3-chloroperbenzoic acid and in the presence of a suitable solvent such as DCM at a temperature between 0° C. and RT.

The compounds of formula Ij can be prepared (Scheme 4) by an aromatic nucleophilic substitution of a compound of formula Ii with a commercially available or freshly prepared solution of sodium alkoxide of formula NaOR¹¹, wherein R¹¹ represents (C₁-C₄)alkyl, in the corresponding alcohol of formula HOR¹¹, the reaction being carried out in the presence of a solvent such as the corresponding alcohol of formula HOR¹¹ or THF at a temperature between 0° C. and 90° C.

The compounds of formula Ik can be prepared (Scheme 4) by an aromatic nucleophilic substitution of a compound of formula Ii with an amine of formula HNR¹¹R¹², wherein R¹¹ represents (C₁-C₄)alkyl and R¹² represents hydrogen or (C₁-C₄)alkyl, optionally in the presence of a suitable base such as NEt₃ or DIPEA and heating in a suitable solvent such as H₂O, THF, CH₃CN or DMF at a temperature between RT and 120° C.

The compound of formula Im can be prepared (Scheme 4) by aromatic nucleophilic substitution of a compound of formula Ii with an aqueous sodium hydroxide solution and heating in a suitable solvent such as dioxane or THF at a temperature between RT and 100° C.

The compound of formula In (or Io, respectively) can be prepared (Scheme 4) by alkylation of a compound of formula Im wherein Y represents NH (or Im wherein Y is different from NH, respectively) using a suitable alkylating reagent such as an alkyl iodide or bromide of formula R¹³—X, wherein R¹³ represents (C₁-C₄)alkyl, in the presence of a suitable base such as Cs₂CO₃ or potassium carbonate and carrying out the reaction in a suitable solvent such as DMF or THF at a temperature between RT and 45° C.

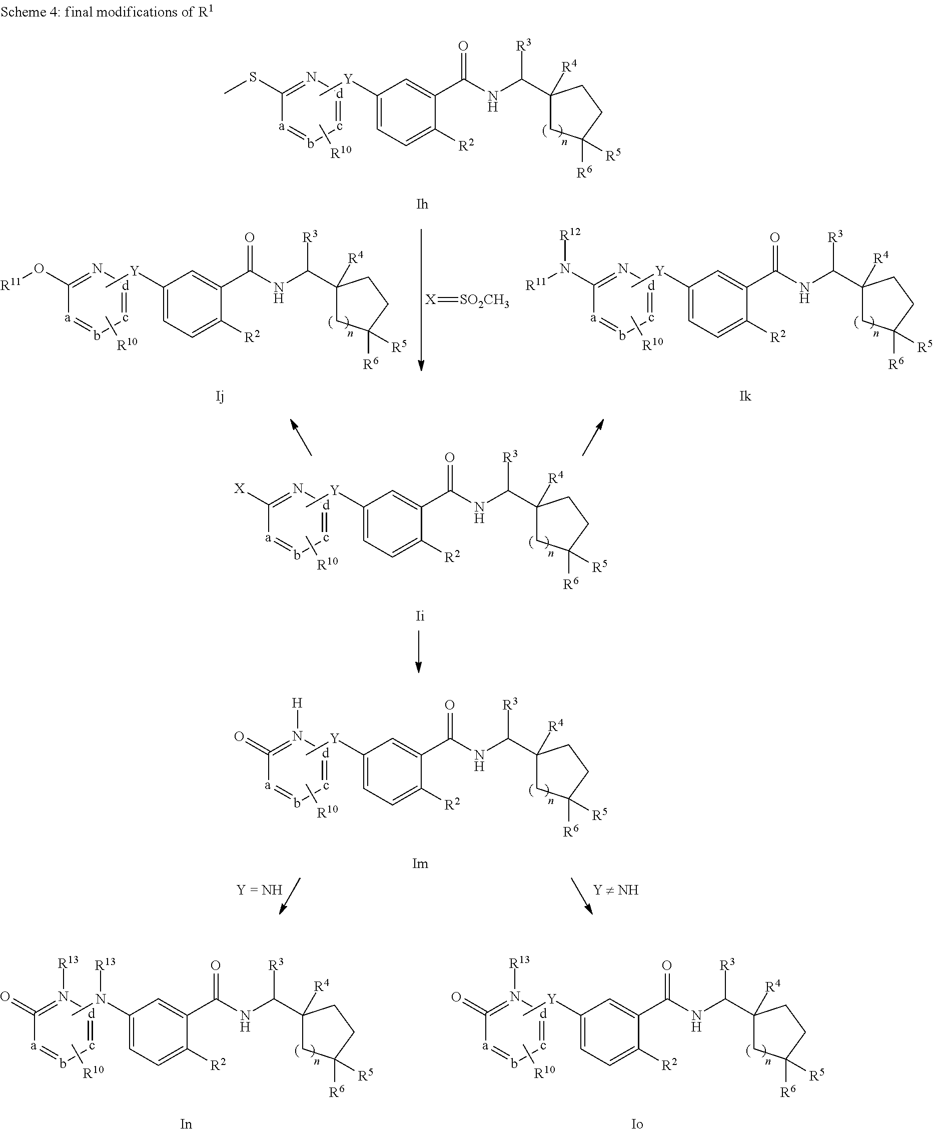

Alternatively compounds of formula Im can be prepared following the procedures outlined in Scheme 5 below. In all the structures of the scheme, one of the letter code, a, b, c or d represents a carbon atom, which is attached to the rest of the molecule via the substituent Y, and the other remaining letter codes are selected from N, CH and CR¹⁰, wherein R¹⁰ represents chloro, to form a 6-membered heteroaryl or a heterocyclyl group R¹ as defined above.

The intermediates of formula XXII and XXIII wherein PG represents a suitable protecting group can be prepared as previously described in Schemes 1 (compounds Ia and Ib), 2 (compounds Ic) and 3 (compounds Ig).

The compounds of formula Im can be prepared (Scheme 5) by cleavage of the protecting group (PG) in intermediates XXII or intermediates XXIII wherein PG represents a trimethylsilylethoxymethyl group by treatment with a tetrabutylammonium fluoride solution in THF and carrying out the reaction in a suitable solvent such as THF at a temperature between RT and 70° C.

Alternatively, the compounds of formula Im can be prepared (Scheme 5) by cleavage of the protecting group (PG) in intermediates XXIII wherein PG represents a trimethylsilylethyl group by treatment with a suitable acid such as TFA and carrying out the reaction in a suitable solvent such as DCM at a temperature around RT.

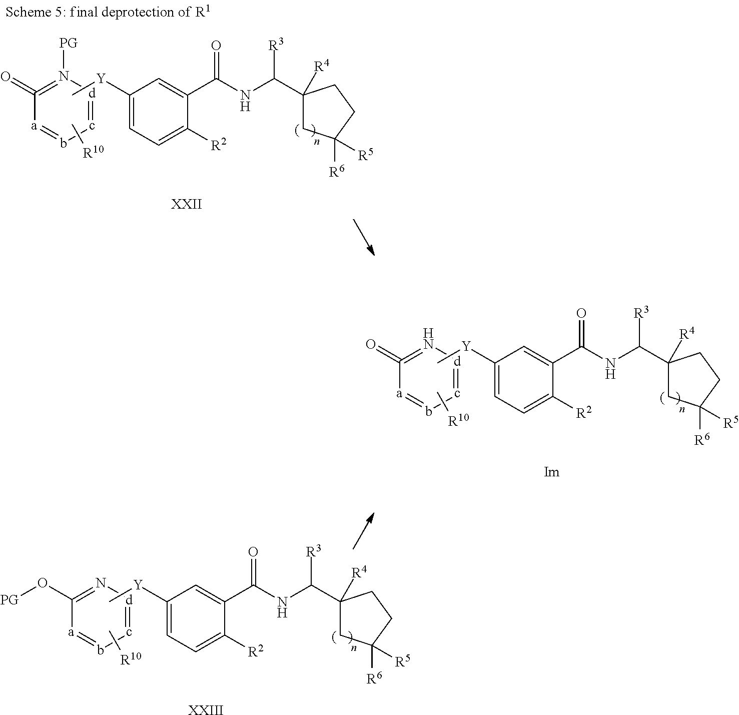

Alternatively compounds of formula Ir and lq can be prepared following the procedures outlined in Scheme 6 below.

The compounds of formula XXIV can be prepared as previously described in Schemes 1 (compounds Ia and Ib), 2 (compound Ic) and 3 (compounds If and Ig) wherein R³ represents H and R⁴ represents COOMe.

The compounds of formula XXV can be prepared (Scheme 6) by hydrolysis of a compound of formula XXIV using standard conditions such as those already described for the synthesis of the compounds of formula III and IV (Scheme 1).

The compounds of formula Iq can be prepared (Scheme 6) by coupling an acid of formula XXV with ammonia as an ethanolic solution using standard amide coupling conditions such as an activation with isobutylchloroformate in the presence of a base such as 4-methylmorpholine and a suitable solvent such as DCM at a temperature between −70° C. to RT.

The compounds of formula Ir can be prepared (Scheme 6) by reduction of a compound of formula XXIV using a suitable reducing reagent such as diisobutylaluminum hydride, in the presence of a suitable solvent such as THF at a temperature around RT.

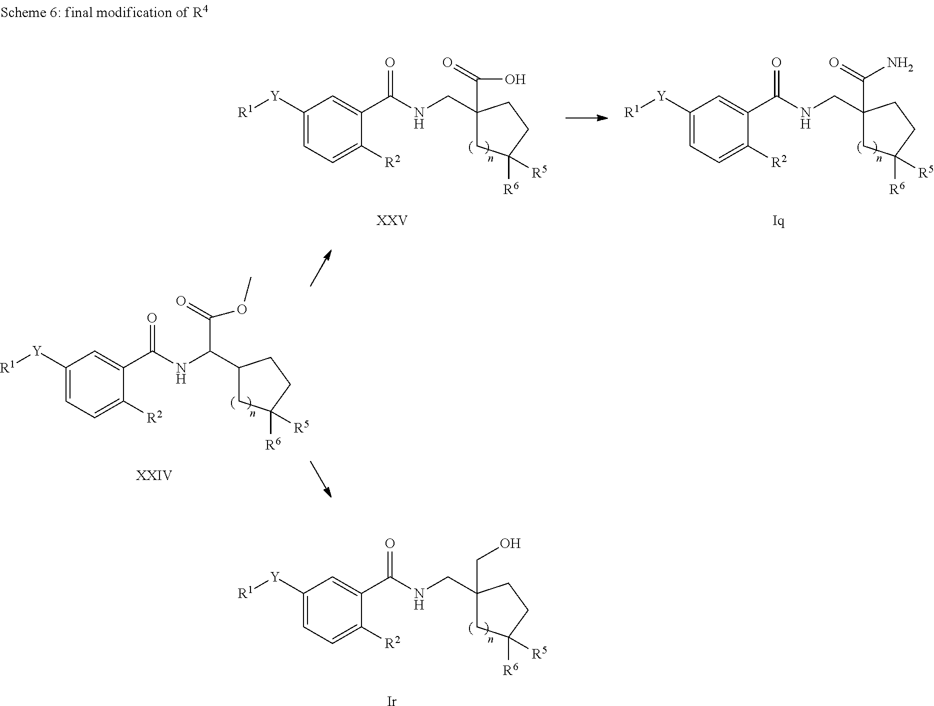

If not commercially available, intermediates of formula R¹—X can be prepared following the procedures outlined in Scheme 7 below. In all the structures of the scheme, one of the letter code, a, b, c or d represents a carbon atom bearing the halogen atom X, and the other remaining letter codes are selected from N, CH and CR¹⁰, wherein R¹⁰ represents chloro, to form a 6-membered heteroaryl or a heterocyclyl group R¹ as defined above.

The compounds of formula XXVII can be prepared (Scheme 7) by alkylation of a compound of formula XXVI using a suitable alkyl halide of formula R¹⁴—X wherein R¹⁴ represents a (C₁-C₄)alkyl or a (C₁-C₂)alkoxy-(C₁-C₄)alkyl group (or of formula PG-X wherein PG represents a trimethylsilylethoxymethyl protecting group, respectively), in the presence of a suitable base such as potassium carbonate and carrying out the reaction in a suitable solvent such as acetone, CH₃CN or DMF at a temperature between RT and 45° C. When PG-X (wherein PG represents a trimethylsilylethoxymethyl protecting group) is used as alkylating reagent, the O-alkylated by-product of formula XXVIII can also be isolated.

Alternatively, the compounds of formula XXVIII, wherein PG represents a trimethylsilylethyl protecting group, can be prepared (Scheme 7) by an aromatic nucleophilic substitution of a compound of formula XXIX with a freshly prepared lithium 2-(trimethylsilyl)ethanolate (by treatment of 2-(trimethylsilyl)ethanol with n-butyllithium in THF at a temperature between −70° C. and −30° C.), the reaction being carried out in the presence of a suitable solvent such as THF at a temperature between −70° C. and RT.

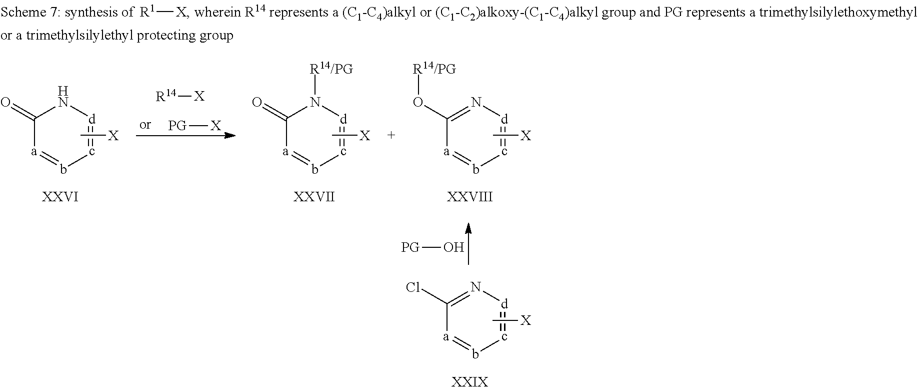

If not commercially available, intermediates of formula II, wherein R³ represents hydrogen and R⁴ represents hydroxy or (C₁-C₄)alkoxy, can be prepared following the procedures outlined in Scheme 8 below.

The compounds of formula XXXII wherein R¹⁵ represents trimethylsilyl or hydrogen can be prepared (Scheme 8) by cyanosilylation of a ketone of formula XXX using a suitable cyanation reagent such as trimethylsilylcyanide, in the presence of a lewis acid such as gold (III) chloride and carrying out the reaction in a suitable solvent such as DCM at a temperature around RT (Synthesis, 2008, 4, 507-510).

The compounds of formula XXXII wherein R¹⁵ represents (C₁-C₄)alkyl can be prepared (Scheme 8) by cyanation of a ketal of formula XXXI with a suitable cyanation reagent such as tert-butyl isocyanide in the presence of a suitable lewis acid such as titanium tetrachloride and carrying out the reaction in a suitable solvent such as DCM at a temperature between −70° C. and RT (Chemistry Lett., 1984, 937-940).

The compounds of formula II, wherein R³ represents hydrogen and R⁴ represents hydroxy or (C₁-C₄)alkoxy, can be prepared (Scheme 8) by reduction of a compound of formula XXXII using a suitable reducing reagent such as lithium aluminum hydride, in the presence of a suitable solvent such as Et₂O or THF at a temperature between 0° C. and RT. In those conditions, the consecutive hydrolysis of the possible trimethylsilyl group R¹⁵ is observed.

The compounds of formula II wherein, R³ represents hydrogen and R⁴ represents hydroxy or (C₁-C₄)alkoxy, can be transformed into their corresponding hydrochloride salt using standard methods.

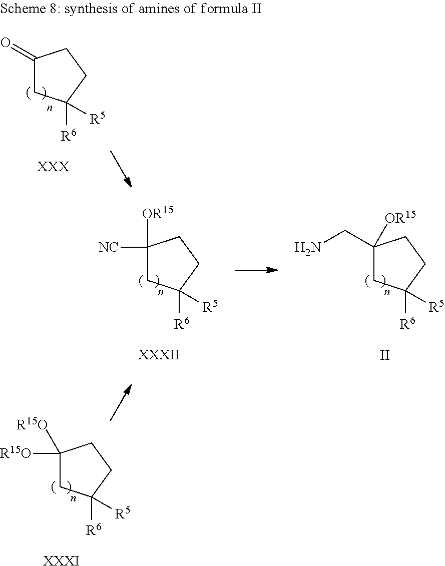

### EXPERIMENTAL PART

### Abbreviations

### As Used Herein and in the Description Above

Ac acetyl
anh. anhydrous
CC column chromatography
DCM dichloromethane
DIPEA diisopropylethylamine
DMF dimethylformamide
DMSO dimethylsulfoxide
Et ethyl
EDC.HCl N-(3-dimethylaminopropyl)-N′-ethylcarbodiimide hydrochloride
eq equivalent
h hour(s)
Hept heptanes
HATU 2-(7-aza-1H-benzotriazole-1-yl)-1,1,3,3-tetramethyluronium hexafluorophosphate
HOBT 1-hydroxybenzotriazole hydrate
HV high vacuum
LC-MS liquid chromatography-mass spectrometry
M molar(ity)
Me methyl
Min minute(s)
NMR nuclear magnetic resonance
ON overnight
PG protecting group
PyBOP benzotriazole-1-yl-oxy-tris-pyrrolidino-phosphonium hexafluorophosphate
PyCloP chlorotripyrrolidinophosphonium hexafluorophosphate
RT room temperature
sat. saturated
TFA trifluoroacetic acid
THF tetrahydrofuran
TBTU O-(benzotriazol-1-yl)-N,N,N′,N′-tetramethyluronium tetrafluoroborate
t_{R} retention time
UV ultra-violet
V is visible

### EXAMPLES

### Characterization Methods Used

NMR: Brucker Avance 400, 400 MHz; chemical shifts are given in ppm relative to the solvent used; multiplicities: s=singlet, d=doublet, t=triplet, q=quadruplet, m=multiplet, br=broad, coupling constants are given in Hz.

LC-MS: Thermo Finnigan MSQ Surveyor MS with Agilent 1100 Binary Pump and DAD. Eluents (acidic conditions): A: H₂O+0.04% TFA; B: CH₃CN; gradient: 5% B→95% B;

runtime: 1.5 min; flow: 4.5 mL/min; detection: UV/Vis+MS, t_{R} is given in min. LC-MS (A): column Zorbax SB-AQ, 5 μm, 4.6×50 mm

LC-MS (B): column Waters XBridge C18, 2.5 μm, 4.6×30 mm

LC-MS (C): column Waters Atlantis T3, 5 μm, 4.6×30 mm;

Eluents (basic conditions): A: H₂O+13 mmol/L NH₄OH; B: CH₃CN; gradient: 5% B→95% B; runtime: 1.5 min; flow: 4.5 mL/min:

LC-MS (D): column Waters XBridge C18, 2.5 μm, 4.6×50 Mm.

### Purification Methods Used

Preparative LC-MS: flow: 75 mL/min. Detection: UV/Vis and/or MS.

Additional information for the purification are summerized in the table below using following explanations:

XBridge: column Waters XBridge C18, 10 μm, 30×75 mm
Atlantis: column Waters Atlantis T3, 10 μm, 30×75 mm
Acidic: eluant: A=H₂O with 0.5% formic acid, B=CH₃CN
Basic: eluant: A=H₂O with 0.125% NH₄OH, B=CH₃CN
Normal gradient: 20% B→95% B over 4 min then 95% B over 2 min
Polar gradient: 10% B→95% B over 4 min then 95% B over 2 min
Very polar gradient: 5% B→50% B over 3 min then 50% B→95% B over 1 min and finally 95% B over 2 min

| | |
|---|---|
| XBridge | Atlantis |

| | acidic | basic | acidic |
|---|---|---|---|
| Normal gradient | Method VI | Method VII | |
| Polar gradient | Method III | Method IV | Method II |
| Very polar | Method I | Method V | |
| gradient | | | |

Column chromatography (CC) was performed using silica gel 60 Merck (0.063-0.200 mm) or using prepacked cartridges (SNAP KP-SIL™, SNAP KP-NH™, Isolute™ Silica II, Isolute™ NH₂ or Isolute™ C¹⁸) from Biotage.

The following examples illustrate the invention but do not at all limit the scope thereof.

### Example 1

### 2-Chloro-N-(1-hydroxy-cyclohexylmethyl)-5-phenylamino-benzamide

### 1.1 2-Chloro-5-phenylamino-benzoic acid methyl ester

Cs₂CO₃ (2.79 g), palladium(II) acetate (82 mg), 2,2′-bis(diphenylphosphino)-1,1′-binaphthalene (228 mg), iodobenzene (0.69 mL) and methyl-5-amino-2-chlorobenzoate (1.13 g) were placed in a flask and flushed with argon. Dioxane (18 mL) was added and the reaction mixture was heated to 100° C. for 24 h. After cooling to RT, it was diluted with Et₂O, filtered over a pad of celite and the filtrate was concentrated in vacuo. The crude material was purified by CC (Hept/EtOAc 1/0 to 8/2) to give 1.18 g of the titled compound as a light yellow solid.

LC-MS (A): t_{R}=1.05 min; [M+H]+: 262.55.

### 1.2 2-Chloro-5-phenylamino-benzoic acid

A solution of intermediate 1.1 (300 mg) in THF (3.44 mL) was treated with a solution of lithium hydroxide hydrate (144 mg) in H₂O (1.15 mL). The reaction mixture was stirred ON at RT, diluted with H₂O and extracted twice with EtOAc. The aqueous phase was acidified with a 1M solution of HCl and extracted three times with EtOAc. The organic phases were dried over MgSO₄ and concentrated in vacuo to give 266 mg of the titled compound as a light yellow solid.

LC-MS (A): t_{R}=0.94 min; [M+CH₃CN+H]+: 289.51.

### 1.3 2-Chloro-N-(1-hydroxy-cyclohexylmethyl)-5-phenylamino-benzamide

To a solution of intermediate 1.2 (200 mg) and DIPEA (0.55 mL) in DCM (1.6 mL) was added HOBT (131 mg) and EDC.HCl (186 mg) at RT. The solution was stirred for 10 min at RT and 1-aminomethyl-cyclohexanol hydrochloride (147 mg) was added. The reaction mixture was further stirred for 18 h at RT and diluted with EtOAc. The organic phase was washed with a 5% solution of KHSO₄, a sat. solution of NaHCO₃ and brine, dried over MgSO₄ and concentrated in vacuo. The crude material was purified by CC (Hept/EtOAc 1/0 to 1/4) to give 244 mg of the titled compound as a light yellow solid.

LC-MS (A): t_{R}=0.94 min; [M+H]+: 359.01.

### Example 2

### 2-Chloro-N-(1-hydroxy-cyclohexyl methyl)-5-(methyl-phenyl-amino)-benzamide

### 2.1 Methyl 2-chloro-5-(methyl(phenyl)amino)benzoate

To a solution of intermediate 1.1 (150 mg) in anh. DMF (1.1 mL) was added Cs₂CO₃ (467 mg) and methyl iodide (0.054 mL). The reaction mixture was stirred for 48 h at 40° C., quenched with H₂O and extracted with EtOAc. The organic phase was dried over MgSO₄ and concentrated in vacuo to give 153 mg of the crude titled compound as a yellow oil.

LC-MS (B): t_{R}=0.96 min; [M+H]+: 276.28.

### 2.2 2-Chloro-5-(methyl(phenyl)amino)benzoic acid

This compound was prepared using a method analogous to that of Example 1 (intermediate 1.2), intermediate 2.1 replacing intermediate 1.1.

LC-MS (B): t_{R}=0.81 min; [M+H]+: 262.21.

### 2.3 2-Chloro-N-((1-hydroxycyclohexyl)methyl)-5-(methyl(phenyl)amino)benzamide

This compound was prepared using a method analogous to that of Example 1 (intermediate 1.3), intermediate 2.2 replacing intermediate 1.2.

LC-MS (B): t_{R}=0.84 min; [M+H]+: 373.20.

### Example 3

### 2-Chloro-N-(1-hydroxy-cyclohexylmethyl)-5-[(2-methoxy-ethyl)-phenyl-amino]-benzamide

This compound was prepared using a method analogous to that of Example 2 (intermediate 2.1), intermediate 1.3 replacing intermediate 1.1 and 2-bromoethyl methyl ether replacing methyl iodide except that the reaction mixture was stirred for 3 days at RT and for 3 days at 70° C. and that the crude was purified by CC (Hept/EtOAc 9/1 to 3/7).

LC-MS (B): t_{R}=0.84 min; [M+H]+: 417.17.

### Example 4

### 2-Chloro-5-(2-fluoro-phenylamino)-N-(1-hydroxy-cyclohexylmethyl)-benzamide

### 4.1 2-Chloro-5-(2-fluoro-phenylamino)-benzoic acid methyl ester

This compound was prepared using a method analogous to that of Example 1 (intermediate 1.1), 1-bromo-2-fluorobenzene replacing iodobenzene.

LC-MS (A): t_{R}=1.01 min; [M+H]+: 279.93

### 4.2 2-Chloro-5-(2-fluoro-phenylamino)-benzoic acid

This compound was prepared using a method analogous to that of Example 1 (intermediate 1.2), intermediate 4.1 replacing intermediate 1.1.

LC-MS (B): t_{R}=0.74 min; [M+CH₃CN+H]+: 307.13

### 4.3 2-Chloro-5-(2-fluoro-phenylamino)-N-(1-hydroxy-cyclohexylmethyl)-benzamide

This compound was prepared using a method analogous to that of Example 1 (intermediate 1.3), intermediate 4.2 replacing intermediate 1.2.

LC-MS (B): t_{R}=0.79 min; [M+H]+: 377.19

### Example 5

### 2-Chloro-5-[(2-fluoro-phenyl)-methyl-amino]-N-(1-hydroxy-cyclohexylmethyl)-benzamide

### 5.1 2-Chloro-5-[(2-fluoro-phenyl)-methyl-amino]-benzoic acid methyl ester

This compound was prepared using a method analogous to that of Example 2 (intermediate 2.1), intermediate 4.1 replacing intermediate 1.1.

LC-MS (B): t_{R}=0.93 min; [M+H]+: 294.18

### 5.2 2-Chloro-5-[(2-fluoro-phenyl)-methyl-amino]-benzoic acid

This compound was prepared using a method analogous to that of Example 1 (intermediate 1.2), intermediate 5.1 replacing intermediate 1.1 except that a 1M solution of NaOH was used instead of lithium hydroxide in H₂O.

LC-MS (B): t_{R}=0.79 min; [M+H]+: 280.22

### 5.3 2-Chloro-5-[(2-fluoro-phenyl)-methyl-amino]-N-(1-hydroxy-cyclohexylmethyl)-benzamide

This compound was prepared using a method analogous to that of Example 1 (intermediate 1.3), intermediate 5.2 replacing intermediate 1.2.

LC-MS (B): t_{R}=0.83 min; [M+H]+: 391.21

### Example 6

### 2-Chloro-5-(2,4-difluoro-phenylamino)-N-(1-hydroxy-cyclohexylmethyl)-benzamide

### 6.1 2-Chloro-5-(2,4-difluoro-phenylamino)-benzoic acid methyl ester

This compound was prepared using a method analogous to that of Example 1 (intermediate 1.1), 2,4-difluoroiodobenzene replacing iodobenzene.

LC-MS (B): t_{R}=0.88 min; [M+CH₃CN+H]+: 339.14

### 6.2 2-Chloro-5-(2,4-difluoro-phenylamino)-benzoic acid

This compound was prepared using a method analogous to that of Example 1 (intermediate 1.2), intermediate 6.1 replacing intermediate 1.1.

LC-MS (B): t_{R}=0.75 min; [M+CH₃CN+H]+: 325.08

### 6.3 2-Chloro-5-(2,4-difluoro-phenylamino)-N-(1-hydroxy-cyclohexylmethyl)-benzamide

This compound was prepared using a method analogous to that of Example 1 (intermediate 1.3), intermediate 6.2 replacing intermediate 1.2.

LC-MS (B): t_{R}=0.79 min; [M+H]+: 395.15

### Example 7

### 2-Chloro-5-[(2,4-difluoro-phenyl)-methyl-amino]-N-(1-hydroxy-cyclohexylmethyl)-benzamide

### 7.1 2-Chloro-5-[(2,4-difluoro-phenyl)-methyl-amino]-benzoic acid methyl ester

This compound was prepared using a method analogous to that of Example 2 (intermediate 2.1), intermediate 6.1 replacing intermediate 1.1.

LC-MS (B): t_{R}=0.94 min; [M+H]+: 312.16

### 7.2 2-Chloro-5-[(2,4-difluoro-phenyl)-methyl-amino]-benzoic acid

This compound was prepared using a method analogous to that of Example 1 (intermediate 1.2), intermediate 7.1 replacing intermediate 1.1.

LC-MS (B): t_{R}=0.81 min; [M+H]+: 298.12

### 7.3 2-Chloro-5-[(2,4-difluoro-phenyl)-methyl-amino]-N-(1-hydroxy-cyclohexylmethyl)-benzamide

This compound was prepared using a method analogous to that of Example 1 (intermediate 1.3), intermediate 7.2 replacing intermediate 1.2.

LC-MS (B): t_{R}=0.85 min; [M+H]+: 409.16

### Example 8

### 2-Chloro-N-(1-hydroxy-cyclohexylmethyl)-5-(pyrimidin-4-ylamino)-benzamide

### 8.1 2-Chloro-5-(pyrimidin-4-ylamino)-benzoic acid methyl ester

Sodium phenoxide (810 mg), tris(dibenzylideneacetone)dipalladium (0) (102 mg), Xantphos (161 mg) and methyl-2-chloro-5-iodobenzoate (1379 mg) were placed in a flask and flushed with argon. 4-aminopyrimidine (486 mg) and dioxane (27.5 mL) were added and the reaction mixture was heated to 120° C. for 18 h. After cooling to RT, it was diluted with EtOAc and washed with a 1M solution of NaOH. The organic phase was dried over MgSO₄ and concentrated in vacuo. The crude material was purified by CC (Hept/EtOAc 1/1 to 0/1) to give 718 mg of the titled compound as a yellowish powder.

LC-MS (B): t_{R}=0.48 min; [M+H]+: 264.21.

### 8.2 2-Chloro-5-(pyrimidin-4-ylamino)-benzoic acid

This compound was prepared using a method analogous to that of Example 1 (intermediate 1.2), intermediate 8.1 replacing intermediate 1.1.

LC-MS (B): t_{R}=0.36 min; [M+H]+: 249.95.

### 8.3 2-Chloro-N-(1-hydroxy-cyclohexylmethyl)-5-(pyrimidin-4-ylamino)-benzamide

This compound was prepared using a method analogous to that of Example 1 (intermediate 1.3), intermediate 8.2 replacing intermediate 1.2, except that the crude was purified by preparative LC-MS using conditions I.

LC-MS (B): t_{R}=0.49 min; [M+H]+: 361.24.

### Example 9

### 2-Chloro-N-(1-hydroxy-cyclohexyl methyl)-5-(methyl-pyrimidin-4-yl-amino)-benzamide

### 9.1 2-Chloro-5-(methyl-pyrimidin-4-yl-amino)-benzoic acid methyl ester

This compound was prepared using a method analogous to that of Example 2 (intermediate 2.1), intermediate 8.1 replacing intermediate 1.1 except that the crude was purified by CC (Hept/EtOAc 1/0 to 0/1).

LC-MS (B): t_{R}=0.48 min; [M+H]+: 278.22

### 9.2 2-Chloro-5-(methyl-pyrimidin-4-yl-amino)-benzoic acid

This compound was prepared using a method analogous to that of Example 1 (intermediate 1.2), intermediate 9.1 replacing intermediate 1.1 except that a 1M solution of NaOH was used instead of lithium hydroxide in H₂O.

LC-MS (B): t_{R}=0.37 min; [M+H]+: 264.24

### 9.3 2-Chloro-N-(1-hydroxy-cyclohexylmethyl)-5-(methyl-pyrimidin-4-yl-amino)-benzamide

This compound was prepared using a method analogous to that of Example 1 (intermediate 1.3), intermediate 9.2 replacing intermediate 1.2.

LC-MS (B): t_{R}=0.49 min; [M+H]+: 375.10

### Example 10

### 2-Chloro-5-(2-chloro-pyrimidin-4-ylamino)-N-(1-hydroxy-cyclohexylmethyl)-benzamide

### 10.1 2-Chloro-5-(2-chloro-pyrimidin-4-ylamino)-benzoic acid methyl ester

This compound was prepared using a method analogous to that of Example 1, intermediate 1.1, 2,4-dichloropyrimidine replacing iodobenzene.

LC-MS (A): t_{R}=0.97 min; [M+H]+: 300.06

### 10.2 2-Chloro-5-(2-chloro-pyrimidin-4-ylamino)-benzoic acid

This compound was prepared using a method analogous to that of Example 1 (intermediate 1.2), intermediate 10.1 replacing intermediate 1.1.

LC-MS (B): t_{R}=0.86 min; [M+CH₃CN+H]+: 325.20.

### 10.3 2-Chloro-5-(2-chloro-pyrimidin-4-ylamino)-N-(1-hydroxy-cyclohexylmethyl)-benzamide

To a solution of intermediate 10.2 (179 mg) and DIPEA (0.431 mL) in DCM (3.2 mL) was added PyCloP (345 mg) and 1-aminomethyl-cyclohexanol hydrochloride (125 mg) at 0° C. The reaction mixture was stirred for 18 h at RT and diluted with DCM. The organic phase was washed with H₂O, sat.-NaHCO₃ and brine, dried over MgSO₄ and concentrated in vacuo. The crude material was purified by CC (EtOAc) to give 122 mg of the titled compound as a white foam.

LC-MS (A): t_{R}=0.89 min; [M+H]+: 394.47.

### Example 11

### 2-Chloro-5-[(2-chloro-pyrimidin-4-yl)-methyl-amino]-N-(1-hydroxy-cyclohexylmethyl)-benzamide

### 11.1 2-Chloro-5-[(2-chloro-pyrimidin-4-yl)-methyl-amino]-benzoic acid methyl ester

To a solution of intermediate 10.1 (1523 mg) in anh. DMF (10.2 mL) was added Cs₂CO₃ (3329 mg) and methyl iodide (0.350 mL). The reaction mixture was stirred for 1 h at RT, quenched with ice H₂O and extracted with EtOAc. The organic phase was dried over MgSO₄ and concentrated in vacuo. The crude material was purified by CC (Hept/EtOAc 1/0 to 1/1) to give 1301 mg of the titled compound as a yellow waxy solid.

LC-MS (A): t_{R}=1.00 min; [M+H]+: 312.12.

### 11.2 2-Chloro-5-[(2-chloro-pyrimidin-4-yl)-methyl-amino]-benzoic acid

This compound was prepared using a method analogous to that of Example 1 (intermediate 1.2), intermediate 11.1 replacing intermediate 1.1.

LC-MS (A): t_{R}=0.87 min; [M+H]+: 297.78.

### 11.3 2-Chloro-5-[(2-chloro-pyrimidin-4-yl)-methyl-amino]-N-(1-hydroxy-cyclohexylmethyl)-benzamide

This compound was prepared using a method analogous to that of Example 10 (intermediate 10.3), intermediate 11.2 replacing intermediate 10.2.

LC-MS (A): t_{R}=0.91 min; [M+H]+: 408.98.

### Example 12

### 2-Chloro-N-(1-hydroxy-cyclohexylmethyl)-5-(2-methoxy-pyrimidin-4-ylamino)-benzamide

Intermediate 10.3 (118 mg) was mixed with a 5.4 M solution of NaOMe in MeOH (0.553 mL) and the reaction mixture was heated to 90° C. for 2 h. It was quenched with H₂O and diluted with EtOAc. The organic phase was washed with a 5% solution of KHSO₄, a sat. solution of NaHCO₃ and brine, dried over MgSO₄ and concentrated in vacuo. The crude material was purified by CC (Hept/EtOAc 1/0 to 65/35) to give 62 mg of the titled compound as a white solid.

LC-MS (A): t_{R}=0.73 min; [M+H]+: 391.15.

### Example 13

### 2-Chloro-N-(1-hydroxy-cyclohexylmethyl)-5-[(2-methoxy-pyrimidin-4-yl)-methyl-amino]-benzamide

This compound was prepared using a method analogous to that of Example 11 (intermediate 11.1), example 12 replacing intermediate 10.1 and the reaction mixture was stirred ON at 40° C.

LC-MS (A): t_{R}=0.74 min; [M+H]+: 405.11.

### Example 14

### 2-Chloro-N-(1-hydroxy-cyclohexylmethyl)-5-[methyl-(2-methylamino-pyrimidin-4-yl)-amino]-benzamide

Intermediate 11.3 (80 mg) was mixed with a 41% solution of methylamine in H₂O (0.165 mL) and the reaction mixture was heated to 80° C. for 2 h. It was quenched with a 1M solution of NaOH and extracted with DCM. The organic phase was dried over MgSO₄ and concentrated in vacuo. The crude material was purified by CC (EtOAc/MeOH 1/0 to 9/1) to give 49 mg of the titled compound as a yellow foam.

LC-MS (A): t_{R}=0.76 min; [M+H]+: 404.11.

### Example 15

### 2-Chloro-5-[(2-dimethylamino-pyrimidin-4-yl)-methyl-amino]-N-(1-hydroxy-cyclohexylmethyl)-benzamide

A solution of intermediate 11.3 (80 mg) in 2-methoxy-ethanol (0.7 mL) was treated with a 40% solution of dimethylamine in H₂O (0.247 mL) and the reaction mixture was heated to 110° C. for 2 h. It was quenched with a 1M solution of NaOH and extracted with DCM. The organic phase was dried over MgSO₄ and concentrated in vacuo. The crude material was purified by CC (EtOAc/MeOH 1/0 to 9/1) to give 63 mg of the titled compound as a white foam.

LC-MS (A): t_{R}=0.78 min; [M+H]+: 417.94.

### Example 16

### 2-Chloro-N-(1-hydroxy-cyclohexylmethyl)-5-(2-hydroxypyrimidin-4-ylamino)-benzamide

### 16.1 2-Chloro-5-((2-hydroxypyrimidin-4-yl)amino)benzoic acid

To a solution of intermediate 10.2 (89 mg) in dioxane (2 mL) was added a 2M solution of NaOH (3.2 mL) at RT and the reaction mixture was stirred for 18 h at 95° C. It was concentrated to dryness and purified by preparative LC-MS using method II.

LC-MS (A): t_{R}=0.58 min; [M+H]+: 265.90.

### 16.2 2-Chloro-N-(1-hydroxy-cyclohexylmethyl)-5-(2-hydroxypyrimidin-4-ylamino)-benzamide

This compound was prepared using a method analogous to that of Example 10 (intermediate 10.3), intermediate 16.1 replacing intermediate 10.2 except that it was purified by preparative LC-MS using method III.

LC-MS (A): t_{R}=0.68 min; [M+H]+: 377.00.

### Example 17

### 2-Chloro-N-(1-hydroxy-cyclohexylmethyl)-5-[methyl-(2-oxo-1,2-dihydro-pyrimidin-4-yl)-amino]-benzamide

This compound was prepared using a method analogous to that of Example 16 (intermediate 16.1), intermediate 11.3 replacing intermediate 10.2 except that it was purified by preparative LC-MS using method III.

LC-MS (A): t_{R}=0.69 min; [M+H]+: 391.60.

### Example 18

### 2-Chloro-N-(1-hydroxy-cyclohexylmethyl)-5-[methyl-(3-methyl-2-oxo-2,3-dihydro-pyrimidin-4-yl)-amino]-benzamide

To a solution of intermediate 16.2 (17 mg) in anh. DMF (0.1 mL) was added Cs₂CO₃ (29 mg) and methyl iodide (0.003 mL). The reaction mixture was stirred for 18 h at RT, quenched with ice H₂O and extracted with EtOAc. The organic phase was dried over MgSO₄ and concentrated in vacuo. The crude was purified by preparative LC-MS using method IV to give 7 mg of titled compound as a white powder.

LC-MS (A): t_{R}=0.72 min; [M+H]+: 405.18.

### Example 19

### 2-Chloro-N-(1-hydroxy-cyclohexylmethyl)-5-[methyl-(1-methyl-2-oxo-1,2-dihydro-pyrimidin-4-yl)-amino]-benzamide

This compound was prepared using a method analogous to that of Example 18 and was isolated as second regioisomer.

LC-MS (A): t_{R}=0.68 min; [M+H]+: 405.04.

### Example 20

### 2-Chloro-5-[(2-chloro-pyrimidin-4-yl)-ethyl-amino]-N-(1-hydroxy-cyclohexylmethyl)-benzamide

### 20.1 2-Chloro-5-[(2-chloro-pyrimidin-4-yl)-ethyl-amino]-benzoic acid methyl ester

This compound was prepared using a method analogous to that of Example 11 (intermediate 11.1), ethyl iodide replacing methyl iodide except that the reaction mixture was stirred for 18 h at RT.

LC-MS (B): t_{R}=0.83 min; [M+H]+: 326.08

### 20.2 2-Chloro-5-[(2-chloro-pyrimidin-4-yl)-ethyl-amino]-benzoic acid

This compound was prepared using a method analogous to that of Example 1 (intermediate 1.2), intermediate 20.1 replacing intermediate 1.1.

LC-MS (B): t_{R}=0.68 min; [M+H]+: 312.09

### 20.3 2-Chloro-5-[(2-chloro-pyrimidin-4-yl)-ethyl-amino]-N-(1-hydroxy-cyclohexylmethyl)-benzamide

This compound was prepared using a method analogous to that of Example 10 (intermediate 10.3), intermediate 20.2 replacing intermediate 10.2.

LC-MS (B): t_{R}=0.73 min; [M+H]+: 423.14

### Example 21

### 2-Chloro-5-[ethyl-(2-oxo-1,2-dihydro-pyrimidin-4-yl)-amino]-N-(1-hydroxy-cyclohexylmethyl)-benzamide

This compound was prepared using a method analogous to that of Example 16 (intermediate 16.1), intermediate 20.3 replacing intermediate 10.2 except that it was purified by preparative LC-MS using method V.

LC-MS (B): t_{R}=0.50 min; [M+H]+: 405.28.

### Example 22

### 2-Chloro-5-[(2-chloro-pyrimidin-4-yl)-propyl-amino]-N-(1-hydroxy-cyclohexylmethyl)-benzamide

### 22.1 2-Chloro-5-[(2-chloro-pyrimidin-4-yl)-propyl-amino]-benzoic acid methyl ester

This compound was prepared using a method analogous to that of Example 11 (intermediate 11.1), propyl iodide replacing methyl iodide except that the reaction mixture was stirred for 18 h at RT.

LC-MS (B): t_{R}=0.89 min; [M+H]+: 340.12

### 22.2 2-Chloro-5-[(2-chloro-pyrimidin-4-yl)-propyl-amino]-benzoic acid

This compound was prepared using a method analogous to that of Example 1 (intermediate 1.2), intermediate 22.1 replacing intermediate 1.1.

LC-MS (B): t_{R}=0.74 min; [M+H]+: 326.09

### 22.3 2-Chloro-5-[(2-chloro-pyrimidin-4-yl)-propyl-amino]-N-(1-hydroxy-cyclohexylmethyl)-benzamide

This compound was prepared using a method analogous to that of Example 10 (intermediate 10.3), intermediate 22.2 replacing intermediate 10.2.

LC-MS (B): t_{R}=0.79 min; [M+H]+: 437.18.

### Example 23

### 2-Chloro-N-(1-hydroxy-cyclohexylmethyl)-5-[(2-oxo-1,2-dihydro-pyrimidin-4-yl)-propyl-amino]-benzamide

This compound was prepared using a method analogous to that of Example 16 (intermediate 16.1), intermediate 22.3 replacing intermediate 10.2 except that it was purified by preparative LC-MS using method V.

LC-MS (B): t_{R}=0.55 min; [M+H]+: 419.29.

### Example 24

### 2-Chloro-5-[(2-chloro-pyrimidin-4-yl)-isopropyl-amino]-N-(1-hydroxy-cyclohexylmethyl)-benzamide

### 24.1 2-Chloro-5-[(2-chloro-pyrimidin-4-yl)-isopropyl-amino]-benzoic acid methyl ester

This compound was prepared using a method analogous to that of Example 11 (intermediate 11.1), isopropyl iodide replacing methyl iodide except that the reaction mixture was stirred for 18 h at RT.

LC-MS (B): t_{R}=0.87 min; [M+H]+: 340.21

### 24.2 2-Chloro-5-[(2-chloro-pyrimidin-4-yl)-isopropyl-amino]-benzoic acid

This compound was prepared using a method analogous to that of Example 1 (intermediate 1.2), intermediate 24.1 replacing intermediate 1.1.

LC-MS (B): t_{R}=0.73 min; [M+H]+: 326.09

### 24.3 2-Chloro-5-[(2-chloro-pyrimidin-4-yl)-isoprooyl-amino]-N-(1-hydroxy-cyclohexylmethyl)-benzamide

This compound was prepared using a method analogous to that of Example 10 (intermediate 10.3), intermediate 24.2 replacing intermediate 10.2.

LC-MS (B): t_{R}=0.78 min; [M+H]+: 437.24.

### Example 25

### 2-Chloro-N-(1-hydroxy-cyclohexylmethyl)-5-[isopropyl-(2-oxo-1,2-dihydro-pyrimidin-4-yl)-amino]-benzamide

This compound was prepared using a method analogous to that of Example 16 (intermediate 16.1), intermediate 24.3 replacing intermediate 10.2 except that it was purified by preparative LC-MS using method V.

LC-MS (B): t_{R}=0.53 min; [M+H]+: 419.29.

### Example 26

### 2-Chloro-5-[(2-chloro-pyrimidin-4-yl)-isobutyl-amino]-N-(1-hydroxy-cyclohexylmethyl)-benzamide

### 26.1 2-Chloro-5-[(2-chloro-pyrimidin-4-yl)-isobutyl-amino]-benzoic acid methyl ester

This compound was prepared using a method analogous to that of Example 11 (intermediate 11.1), 1-iodo-2-methylpropane replacing methyl iodide except that the reaction mixture was stirred for 18 h at RT and for 18 h at 40° C.

LC-MS (B): t_{R}=0.93 min; [M+H]+: 353.98

### 26.2 2-Chloro-5-[(2-chloro-pyrimidin-4-yl)-isobutyl-amino]-benzoic acid

This compound was prepared using a method analogous to that of Example 1 (intermediate 1.2), intermediate 26.1 replacing intermediate 1.1 except that a 2M solution of NaOH was used instead of lithium hydroxide in H₂O.

LC-MS (B): t_{R}=0.79 min; [M+H]+: 340.13

### 26.3 2-Chloro-5-[(2-chloro-pyrimidin-4-yl)-isobutyl-amino]-N-(1-hydroxy-cyclohexylmethyl)-benzamide

This compound was prepared using a method analogous to that of Example 10 (intermediate 10.3), intermediate 26.2 replacing intermediate 10.2.

LC-MS (B): t_{R}=0.83 min; [M+H]+: 451.27.

### Example 27

### 2-Chloro-N-(1-hydroxy-cyclohexylmethyl)-5-[isobutyl-(2-oxo-1,2-dihydro-pyrimidin-4-yl)-amino]-benzamide

This compound was prepared using a method analogous to that of Example 16 (intermediate 16.1), intermediate 26.3 replacing intermediate 10.2 except that it was purified by CC (EtOAc/MeOH 1/0 to 8/2).

LC-MS (B): t_{R}=0.58 min; [M+H]+: 433.18.

### Example 28

### 2-Chloro-N-(1-hydroxy-cyclohexylmethyl)-5-[isobutyl-(2-methoxy-pyrimidin-4-yl)-amino]-benzamide

This compound was prepared using a method analogous to that of Example 12, intermediate 26.3 replacing intermediate 10.3 except that the reaction mixture was stirred for 45 min at 50° C.

LC-MS (B): t_{R}=0.63 min; [M+H]+: 447.35.

### Example 29

### 5-[Benzyl-(2-chloro-pyrimidin-4-yl)-amino]-2-chloro-N-(1-hydroxy-cyclohexylmethyl)-benzamide

### 29.1 5-[Benzyl-(2-chloro-pyrimidin-4-yl)-amino]-2-chloro-benzoic acid methyl ester

This compound was prepared using a method analogous to that of Example 11 (intermediate 11.1), benzyl bromide replacing methyl iodide except that the reaction mixture was stirred for 18 h at RT.

LC-MS (B): t_{R}=0.91 min; [M+H]+: 388.20

### 29.2 5-[Benzyl-(2-chloro-pyrimidin-4-yl)-amino]-2-chloro-benzoic acid

This compound was prepared using a method analogous to that of Example 1 (intermediate 1.2), intermediate 29.1 replacing intermediate 1.1 except that a 2M solution of NaOH was used instead of lithium hydroxide in H₂O.

LC-MS (B): t_{R}=0.79 min; [M+H]+: 374.03

### 29.3 5-[Benzyl-(2-chloro-pyrimidin-4-yl)-amino]-2-chloro-N-(1-hydroxy-cyclohexylmethyl)-benzamide

This compound was prepared using a method analogous to that of Example 10 (intermediate 10.3), intermediate 29.2 replacing intermediate 10.2.

LC-MS (B): t_{R}=0.83 min; [M+H]+: 485.26.

### Example 30

### 5-[Benzyl-(2-oxo-1,2-dihydro-pyrimidin-4-yl)-amino]-2-chloro-N-(1-hydroxy-cyclohexylmethyl)-benzamide

This compound was prepared using a method analogous to that of Example 16 (intermediate 16.1), intermediate 29.3 replacing intermediate 10.2 except that it was purified by CC (EtOAc/MeOH 1/0 to 8/2).

LC-MS (B): t_{R}=0.62 min; [M+H]+: 467.33.

### Example 31

### 5-[Benzyl-(2-methoxy-pyrimidin-4-yl)-amino]-2-chloro-N-(1-hydroxy-cyclohexylmethyl)-benzamide

This compound was prepared using a method analogous to that of Example 12, intermediate 29.3 replacing intermediate 10.3 except that the reaction mixture was stirred for 45 min at 50° C.

LC-MS (B): t_{R}=0.64 min; [M+H]+: 481.15.

### Example 32

### 2-Chloro-5-[(2-chloro-pyrimidin-4-yl)-(2-methoxy-ethyl)-amino]-N-(1-hydroxy-cyclohexylmethyl)-benzamide

### 32.1 2-Chloro-5-[(2-chloro-pyrimidin-4-yl)-(2-methoxy-ethyl)-amino]-benzoic acid methyl ester

This compound was prepared using a method analogous to that of Example 11 (intermediate 11.1), 2-bromoethyl methyl ether replacing methyl iodide except that the reaction mixture was stirred for 18 h at 40° C.

LC-MS (B): t_{R}=0.79 min; [M+H]+: 356.22

### 32.2 2-Chloro-5-[(2-chloro-pyrimidin-4-yl)-(2-methoxy-ethyl)-amino]-benzoic acid

This compound was prepared using a method analogous to that of Example 1 (intermediate 1.2), intermediate 32.1 replacing intermediate 1.1 except that a 2M solution of NaOH was used instead of lithium hydroxide in H₂O.

LC-MS (B): t_{R}=0.65 min; [M+H]+: 342.12

### 32.3 2-Chloro-5-[(2-chloro-pyrimidin-4-yl)-(2-methoxy-ethyl)-amino]-N-(1-hydroxy-cyclohexylmethyl)-benzamide

This compound was prepared using a method analogous to that of Example 10 (intermediate 10.3), intermediate 32.2 replacing intermediate 10.2.

LC-MS (B): t_{R}=0.71 min; [M+H]+: 453.24

### Example 33

### 2-Chloro-N-(1-hydroxy-cyclohexylmethyl)-5-[(2-methoxy-ethyl)-(2-oxo-1,2-dihydro-pyrimidin-4-yl)-amino]-benzamide

This compound was prepared using a method analogous to that of Example 16 (intermediate 16.1), intermediate 32.3 replacing intermediate 10.2 except that it was purified by CC (EtOAc/MeOH 1/0 to 8/2).

LC-MS (B): t_{R}=0.52 min; [M+H]+: 435.27.

### Example 34

### 2-Chloro-N-(1-hydroxy-cyclohexylmethyl)-5-[(2-methoxy-ethyl)-(2-methoxy-pyrimidin-4-yl)-amino]-benzamide

This compound was prepared using a method analogous to that of Example 12, intermediate 32.3 replacing intermediate 10.3 except that the reaction mixture was stirred for 18 h at 30° C.

LC-MS (B): t_{R}=0.54 min; [M+H]+: 449.11.

### Example 35

### 2-Chloro-5-[(2-chloro-pyrimidin-4-yl)-cyclopentylmethyl-amino]-N-(1-hydroxy-cyclohexylmethyl)-benzamide

### 35.1 2-Chloro-5-[(2-chloro-pyrimidin-4-yl)-cyclopentylmethyl-amino]-benzoic acid methyl ester

This compound was prepared using a method analogous to that of Example 11 (intermediate 11.1), iodomethylcyclopentane replacing methyl iodide except that the reaction mixture was stirred for 4 days at 40° C.

LC-MS (B): t_{R}=1.00 min; [M+H]+: 380.07

### 35.2 2-Chloro-5-[(2-chloro-pyrimidin-4-yl)-cyclopentylmethyl-amino]-benzoic acid

This compound was prepared using a method analogous to that of Example 1 (intermediate 1.2), intermediate 35.1 replacing intermediate 1.1 except that a 2M solution of NaOH was used instead of lithium hydroxide in H₂O.

LC-MS (B): t_{R}=0.86 min; [M+H]+: 366.08

### 35.3 2-Chloro-5-[(2-chloro-pyrimidin-4-yl)-cyclopentylmethyl-amino]-N-(1-hydroxy-cyclohexylmethyl)-benzamide

This compound was prepared using a method analogous to that of Example 10 (intermediate 10.3), intermediate 35.2 replacing intermediate 10.2.

LC-MS (B): t_{R}=0.90 min; [M+H]+: 477.29

### Example 36

### 2-Chloro-5-[cyclopentylmethyl-(2-oxo-1,2-dihydro-pyrimidin-4-yl)-amino]-N-(1-hydroxy-cyclohexylmethyl)-benzamide

This compound was prepared using a method analogous to that of Example 16 (intermediate 16.1), intermediate 35.3 replacing intermediate 10.2 except that it was purified by CC (EtOAc/MeOH 1/0 to 8/2).

LC-MS (B): t_{R}=0.63 min; [M+H]+: 459.37.

### Example 37

### 2-Chloro-5-[cyclopentylmethyl-(2-methoxy-pyrimidin-4-yl)-amino]-N-(1-hydroxy-cyclohexylmethyl)-benzamide

This compound was prepared using a method analogous to that of Example 12, intermediate 35.3 replacing intermediate 10.3 except that the reaction mixture was stirred for 2 h at 50° C.

LC-MS (B): t_{R}=0.68 min; [M+H]+: 473.36.

### Example 38

### 2-Chloro-5-[(2-chloro-pyrimidin-4-yl)-(2-phenoxy-ethyl)-amino]-N-(1-hydroxy-cyclohexylmethyl)-benzamide

### 38.1 2-Chloro-5-[(2-chloro-pyrimidin-4-yl)-(2-phenoxy-ethyl)-amino]-benzoic acid methyl ester

This compound was prepared using a method analogous to that of Example 11 (intermediate 11.1), 2-bromoethyl phenyl ether replacing methyl iodide except that the reaction mixture was stirred for 3 days at RT.

LC-MS (B): t_{R}=0.94 min; [M+H]+: 417.86

### 38.2 2-Chloro-5-[(2-chloro-pyrimidin-4-yl)-(2-phenoxy-ethyl)-amino]-benzoic acid

This compound was prepared using a method analogous to that of Example 1 (intermediate 1.2), intermediate 38.1 replacing intermediate 1.1 except that a 2M solution of NaOH was used instead of lithium hydroxide in H₂O.

LC-MS (B): t_{R}=0.82 min; [M+H]+: 404.04

### 38.3 2-Chloro-5-[(2-chloro-pyrimidin-4-yl)-(2-phenoxy-ethyl)-amino]-N-(1-hydroxy-cyclohexylmethyl)-benzamide

This compound was prepared using a method analogous to that of Example 10 (intermediate 10.3), intermediate 38.2 replacing intermediate 10.2.

LC-MS (B): t_{R}=0.86 min; [M+H]+: 515.28

### Example 39

### 2-Chloro-N-(1-hydroxy-cyclohexylmethyl)-5-[(2-oxo-1,2-dihydro-pyrimidin-4-yl)-(2-phenoxy-ethyl)-amino]-benzamide

This compound was prepared using a method analogous to that of Example 16 (intermediate 16.1), intermediate 38.3 replacing intermediate 10.2 except that it was purified by CC (EtOAc/MeOH 1/0 to 8/2).

LC-MS (B): t_{R}=0.65 min; [M+H]+: 497.15.

### Example 40

### 2-Chloro-N-(1-hydroxy-cyclohexyl methyl)-5-[(2-methoxy-pyrimidin-4-yl)-(2-phenoxy-ethyl)-amino]-benzamide

This compound was prepared using a method analogous to that of Example 12, intermediate 38.3 replacing intermediate 10.3 except that the reaction mixture was stirred for 18 h at 30° C.

LC-MS (B): t_{R}=0.67 min; [M+H]+: 511.31.

### Example 41

### 2-Chloro-5-[(2-chloro-pyrimidin-4-yl)-methyl-amino]-N—((R)-1-cyclohexyl-ethyl)-benzamide

This compound was prepared using a method analogous to that of Example 10 (intermediate 10.3), intermediate 11.2 replacing intermediate 10.2 and (R)-(−)-1-cyclohexylethylamine replacing 1-aminomethyl-cyclohexanol hydrochloride.

LC-MS (B): t_{R}=0.88 min; [M+H]+: 407.18.

### Example 42

### 2-Chloro-N—((R)-1-cyclohexyl-ethyl)-5-[methyl-(2-oxo-1,2-dihydro-pyrimidin-4-yl)-amino]-benzamide

This compound was prepared using a method analogous to that of Example 16 (intermediate 16.1), example 41 replacing intermediate 10.2 except that it was purified by preparative LC-MS using method V.

LC-MS (B): t_{R}=0.64 min; [M+H]+: 389.22.

### Example 43

### 2-Chloro-5-[(2-chloro-pyrimidin-4-yl)-methyl-amino]-N—((S)-1-cyclohexyl-2-hydroxy-ethyl)-benzamide

This compound was prepared using a method analogous to that of Example 10 (intermediate 10.3), intermediate 11.2 replacing intermediate 10.2 and L-cyclohexylglycinol replacing 1-aminomethyl-cyclohexanol hydrochloride.

LC-MS (B): t_{R}=0.73 min; [M+H]+: 423.18.

### Example 44

### 2-Chloro-N—((S)-1-cyclohexyl-2-hydroxy-ethyl)-5-[methyl-(2-oxo-1,2-dihydro-pyrimidin-4-yl)-amino]-benzamide

This compound was prepared using a method analogous to that of Example 16 (intermediate 16.1), example 43 replacing intermediate 10.2 except that it was purified by preparative LC-MS using method I.

LC-MS (D): t_{R}=0.69 min; [M+H]+: 405.19.

### Example 45

### 2-Chloro-5-[(2-chloro-pyrimidin-4-yl)-methyl-amino]-N-(1-hydroxy-cycloheptylmethyl)-benzamide

### 45.1 1-Trimethylsilanyloxy-cycloheptanecarbonitrile

To a solution of cycloheptanone (5 g) in anh. DCM (106 mL) were added trimethylsilyl cyanide (5.14 g) and gold (III) chloride (130 mg) at RT. The reaction mixture was stirred for 1 h at RT, quenched with a 10% solution of Na₂CO₃ and extracted with DCM. The organic phases was dried over MgSO₄ and concentrated in vacuo. The crude was purified by CC (Hept/EtOAc 1/0 to 65/35) to give 4.93 g of titled compound as a colorless oil.

¹H NMR (CDCl₃) δ: 2.12 (dd, J₁=14.1 Hz, J₂=8.4 Hz, 2H), 1.95 (m, 2H), 1.65 (m, 8H), 0.26 (s, 9H)

### 45.2 1-Aminomethyl-cycloheptanol hydrochloride

A 2M solution of lithium aluminum hydride (6.13 mL) in THF was diluted with anh. Et₂O (6.5 mL) and a solution of 1-trimethylsilanyloxy-cycloheptanecarbonitrile (1.73 g) in anh. Et₂O (3.3 mL) was added dropwise for 15 min at 0° C. The ice bath was removed and the reaction mixture was stirred for 2 h at RT. It was cooled to 0° C. and quenched successively with ice H₂O (1 mL), a 1M solution of NaOH (1 mL) and ice H₂O (3.3 mL). The mixture was stirred for 10 min at RT, diluted with Et₂O and filtered over a pad of celite. The filtrate was concentrated in vacuo and the residue was redissolved in Et₂O (13 mL) and few drops of dioxane. A 4M solution of hydrogen chloride in dioxane (6.6 mL) was added at RT and the precipitate was filtered to give 1.05 g of the 1-aminomethyl-cycloheptanol hydrochloride as a white solid.

¹H NMR (CD₃OD) δ: 2.90 (s, 2H), 1.59 (m, 12H)

¹³C NMR (CD₃OD) δ: 72.1, 47.7, 38.1, 29.6, 21.7

### 45.3 2-Chloro-5-[(2-chloro-pyrimidin-4-yl)-methyl-amino]-N-(1-hydroxy-cycloheptylmethyl)-benzamide

This compound was prepared using a method analogous to that of Example 10 (intermediate 10.3), intermediate 11.2 replacing intermediate 10.2 and intermediate 45.2 replacing 1-aminomethyl-cyclohexanol hydrochloride.

LC-MS (B): t_{R}=0.73 min; [M+H]+: 423.17.

### Example 46

### 2-Chloro-N-(1-hydroxy-cycloheptylmethyl)-5-[methyl-(2-oxo-1,2-dihydro-pyrimidin-4-yl)-amino]-benzamide

This compound was prepared using a method analogous to that of Example 16 (intermediate 16.1), intermediate 45.3 replacing intermediate 10.2 except that the reaction mixture was stirred for 72 h at 80° C. and the crude was purified by preparative LC-MS using method I and then by CC (EtOAc/MeOH 1/0 to 1/1).

LC-MS (B): t_{R}=0.52 min; [M+H]+: 405.27.

### Example 47

### 2-Chloro-5-[(2-chloro-pyrimidin-4-yl)-methyl-amino]-N-(1-hydroxy-cyclooctylmethyl)-benzamide

### 47.1 1-Trimethylsilanyloxy-cyclooctanecarbonitrile

This compound was prepared using a method analogous to that of Example 45 (intermediate 45.1), cyclooctanone replacing cycloheptanone except that the reaction mixture was stirred for 2 h at RT.

¹H NMR (CDCl₃) δ: 2.04 (t, J=5.6 Hz, 4H), 1.63 (m, 10H), 0.26 (m, 9H)

### 47.2 1-Aminomethyl-cyclooctanol hydrochloride

This compound was prepared using a method analogous to that of Example 45 (intermediate 45.2), intermediate 47.1 replacing intermediate 45.1 except that the reaction mixture was stirred for 2 h at RT.

¹H NMR ((CD₃)₂SO) δ: 7.87 (s, 3H), 4.82 (s, 1H), 2.70 (s, 2H), 1.54 (m, 14H)

### 47.3 2-Chloro-5-[(2-chloro-pyrimidin-4-yl)-methyl-amino]-N-(1-hydroxy-cyclooctylmethyl)-benzamide

This compound was prepared using a method analogous to that of Example 10 (intermediate 10.3), intermediate 11.2 replacing intermediate 10.2 and intermediate 47.2 replacing 1-aminomethyl-cyclohexanol hydrochloride.

LC-MS (B): t_{R}=0.77 min; [M+H]+: 437.22.

### Example 48

### 2-Chloro-N-(1-hydroxy-cyclooctylmethyl)-5-[methyl-(2-oxo-2,3-dihydro-pyrimidin-4-yl)-amino]-benzamide

This compound was prepared using a method analogous to that of Example 16 (intermediate 16.1), intermediate 47.3 replacing intermediate 10.2 except that the reaction mixture was stirred for 6 h at 90° C. and the crude was purified by preparative LC-MS using method I.

LC-MS (B): t_{R}=0.57 min; [M+H]+: 419.22.

### Example 49

### 2-Chloro-5-[(2-chloro-pyrimidin-4-yl)-methyl-amino]-N-(1-hydroxy-cyclopentylmethyl)-benzamide

### 49.1 1-Hydroxy-cyclopentanecarbonitrile

This compound was prepared using a method analogous to that of Example 45 (intermediate 45.1), cyclopentanone replacing cycloheptanone except that the reaction mixture was stirred for 2 h at RT and the desilylated product was isolated.

¹H NMR ((CD₃)₂SO) δ: 6.25 (s, 1H), 1.93 (m, 4H), 1.71 (m, 4H)

¹³C NMR ((CD₃)₂SO) δ: 123.8, 72.3, 40.0, 23.1

### 49.2 1-Aminomethyl-cyclopentanol hydrochloride

This compound was prepared using a method analogous to that of Example 45 (intermediate 45.2), intermediate 49.1 replacing intermediate 45.1.

¹H NMR ((CD₃)₂SO) δ: 7.99 (s, 3H), 4.94 (s, 1H), 2.83 (d, J=4.9 Hz, 2H), 1.63 (m, 8H)

¹³C NMR ((CD₃)₂SO) δ: 78.7, 47.9, 37.7, 23.8

### 49.3 2-Chloro-5-[(2-chloro-pyrimidin-4-yl)-methyl-amino]-N-(1-hydroxy-cyclopentylmethyl)-benzamide

This compound was prepared using a method analogous to that of Example 10 (intermediate 10.3), intermediate 11.2 replacing intermediate 10.2 and intermediate 49.2 replacing 1-aminomethyl-cyclohexanol hydrochloride.

LC-MS (B): t_{R}=0.63 min; [M+H]+: 395.04.

### Example 50

### 2-Chloro-N-(1-hydroxy-cyclopentylmethyl)-5-[methyl-(2-oxo-2,3-dihydro-pyrimidin-4-yl)-amino]-benzamide

This compound was prepared using a method analogous to that of Example 16 (intermediate 16.1), intermediate 49.3 replacing intermediate 10.2 except that the reaction mixture was stirred for 18 h at 80° C. and the crude was purified by preparative LC-MS using method IV.

LC-MS (B): t_{R}=0.43 min; [M+H]+: 377.10.

### Example 51

### 2-Chloro-5-[(2-chloro-pyrimidin-4-yl)-methyl-amino]-N-(1-methoxy-cyclohexylmethyl)-benzamide

### 51.1 1-methoxycyclohexanecarbonitrile

To a solution of 1,1-dimethoxycyclohexane (3 g) in DCM (62 mL) was added dropwise titanium tetrachloride (2.28 mL). The reaction mixture was cooled to −70° C. and tert-butyl isocyanide (2.35 mL) was added dropwise. The mixture was stirred for 5 min at −70° C. then allowed to warm up ON to RT. It was quenched with a sat. solution of NaHCO₃ and extracted with DCM. The organic phase was washed with brine, dried over MgSO₄ and concentrated in vacuo. The crude was purified by CC (Hept/EtOAc 1/0 to 9/1) to give 1 g of the titled compound as an orange oil.

¹H NMR ((CD₃)₂SO) δ: 3.08 (s, 3H), 1.58 (m, 10H)

### 51.2 (1-methoxycyclohexyl)methanamine

This compound was prepared using a method analogous to that of Example 45 (intermediate 45.2), intermediate 51.1 replacing intermediate 45.1 except that the compound was isolated as a free amine.

¹H NMR ((CD₃)₂SO) δ: 3.04 (s, 3H), 2.46 (d, J=12.3 Hz, 2H), 1.42 (m, 10H)

### 51.3 2-Chloro-5-[(2-chloro-pyrimidin-4-yl)-methyl-amino]-N-(1-methoxy-cyclohexylmethyl)-benzamide

This compound was prepared using a method analogous to that of Example 10 (intermediate 10.3), intermediate 11.2 replacing intermediate 10.2 and intermediate 51.2 replacing 1-aminomethyl-cyclohexanol hydrochloride.

LC-MS (B): t_{R}=0.81 min; [M+H]+: 423.16.

### Example 52

### 2-Chloro-N-(1-methoxy-cyclohexylmethyl)-5-[methyl-(2-oxo-2,3-dihydro-pyrimidin-4-yl)-amino]-benzamide

This compound was prepared using a method analogous to that of Example 16 (intermediate 16.1), intermediate 51.3 replacing intermediate 10.2 except that the reaction mixture was stirred for 72 h at 80° C. and the crude was purified by preparative LC-MS using method VII.

LC-MS (B): t_{R}=0.57 min; [M+H]+: 405.22.

### Example 53

### N-(1-Carbamoyl-cyclohexylmethyl)-2-chloro-5-[(2-chloro-pyrimidin-4-yl)-methyl-amino]-benzamide

### 53.1 1-({2-Chloro-5-[(2-chloro-pyrimidin-4-yl)-methyl-amino]-benzoylamino}-methyl)-cyclohexanecarboxylic acid methyl ester

This compound was prepared using a method analogous to that of Example 10 (intermediate 10.3), intermediate 11.2 replacing intermediate 10.2 and methyl 1-aminomethyl-cyclohexanecarboxylate replacing 1-aminomethyl-cyclohexanol hydrochloride.

LC-MS (B): t_{R}=0.82 min; [M+H]+: 451.22.

### 53.2 1-({2-Chloro-5-[(2-chloro-pyrimidin-4-yl)-methyl-amino]-benzoylamino}-methyl)-cyclohexanecarboxylic acid

This compound was prepared using a method analogous to that of Example 1 (intermediate 1.2), intermediate 53.1 replacing intermediate 1.1.

LC-MS (B): t_{R}=0.71 min; [M+H]+: 437.19.

### 53.3 N-(1-Carbamoyl-cyclohexylmethyl)-2-chloro-5-[(2-chloro-pyrimidin-4-yl)-methyl-amino]-benzamide

To a solution of intermediate 53.2 (70 mg) in DCM (1 mL) was added 4-methylmorpholine (0.020 mL) and isobutyl chloroformate (0.022 mL) at −70° C. The mixture was stirred for 10 min at −70° C. and a 0.5M solution of ammonia in EtOH (0.320 mL) was added dropwise. The reaction mixture was allowed to warm up ON to RT, quenched with H₂O and extracted with EtOAc. The organic phase was washed with a sat. solution of NaHCO₃, with brine, dried over MgSO₄ and concentrated in vacuo. The crude was purified by CC (Hept/EtOAc 1/0 to 0/1) to give 18 mg of the titled compound as a white solid.

LC-MS (B): t_{R}=0.63 min; [M+H]+: 436.22.

### Example 54

### N-(1-Carbamoyl-cyclohexylmethyl)-2-chloro-5-[methyl-(2-oxo-2,3-dihydro-pyrimidin-4-yl)-amino]-benzamide

This compound was prepared using a method analogous to that of Example 16 (intermediate 16.1), intermediate 53.3 replacing intermediate 10.2 except that the reaction mixture was stirred for 72 h at 80° C. and the crude was purified by preparative LC-MS using method IV.

LC-MS (B): t_{R}=0.46 min; [M+H]+: 417.99.

### Example 55

### 2-Chloro-5-[(2-chloro-pyrimidin-4-yl)-methyl-amino]-N-(1-hydroxymethyl-cyclohexylmethyl)-benzamide

To a solution of intermediate 53.1 (150 mg) in THF (1 mL) was added a 1M solution of diisobutylaluminum hydride (0.830 mL) at 0° C. and the reaction mixture was stirred at RT. Additional amounts of a 1M solution of diisobutylaluminum hydride (3×0.665 mL) were necessary until completion of the reaction. The reaction mixture was quenched with H₂O and extracted with EtOAc. The organic phase was washed with sat. solution of NaHCO₃ and brine, dried over MgSO₄ and concentrated in vacuo. The crude was purified by CC (Hept/EtOAc 1/0 to 3/7) to give 56 mg of the titled compound as a white foam.

LC-MS (B): t_{R}=0.75 min; [M+H]+: 423.21.

### Example 56

### 2-Chloro-N-(1-hydroxymethyl-cyclohexylmethyl)-5-[methyl-(2-oxo-2,3-dihydro-pyrimidin-4-yl)-amino]-benzamide

This compound was prepared using a method analogous to that of Example 16 (intermediate 16.1), example 55 replacing intermediate 10.2 except that the reaction mixture was stirred for 18 h at 90° C. and the crude was purified by preparative LC-MS using method III.

LC-MS (B): t_{R}=0.54 min; [M+H]+: 405.16.

### Example 57

### 2-Chloro-5-[(2-chloro-pyrimidin-4-yl)-methyl-amino]-N-(4,4-difluoro-1-hydroxy-cyclohexylmethyl)-benzamide

### 57.1 4,4-Difluoro-1-trimethylsilanyloxy-cyclohexanecarbonitrile

To a solution of 4,4-difluorocyclohexanone (967 mg) in anh. DCM (18 mL) were added zinc iodide (23 mg) and trimethylsilyl cyanide (1.1 mL) at 0° C. The reaction mixture was stirred for 1 h at RT, quenched with a 10% solution of Na₂CO₃ and extracted with DCM. The organic phases was dried over MgSO₄ and concentrated in vacuo to give 1.5 g of titled compound as a colorless oil.

¹H NMR ((CD₃)₂SO) δ: 2.04 (m, 8H), 0.25 (m, 9H)

### 57.2 1-Aminomethyl-4,4-difluoro-cyclohexanol hydrochloride

This compound was prepared using a method analogous to that of Example 45 (intermediate 45.2), intermediate 57.1 replacing intermediate 45.1.

¹H NMR ((CD₃)₂SO) δ: 8.04 (s, 3H), 5.21 (s, 1H), 2.82 (s, 2H), 1.98 (m, 4H), 1.74 (m, 2H), 1.55 (td, J₁=13.2 Hz, J₂=4.1 Hz, 2H)

### 57.3 2-Chloro-5-[(2-chloro-pyrimidin-4-yl)-methyl-amino-]-N-(4,4-difluoro-1-hydroxy-cyclohexylmethyl)-benzamide

This compound was prepared using a method analogous to that of Example 10 (intermediate 10.3), intermediate 11.2 replacing intermediate 10.2 and intermediate 57.2 replacing 1-aminomethyl-cyclohexanol hydrochloride.

LC-MS (B): t_{R}=0.69 min; [M+H]+: 445.09.

### Example 58

### 2-Chloro-N-(4,4-difluoro-1-hydroxy-cyclohexylmethyl)-5-[methyl-(2-oxo-2,3-dihydro-pyrimidin-4-yl)-amino]-benzamide

This compound was prepared using a method analogous to that of Example 16 (intermediate 16.1), intermediate 57.3 replacing intermediate 10.2 except that the reaction mixture was stirred for 18 h at 90° C. and the crude was purified by preparative LC-MS using method IV.

LC-MS (B): t_{R}=0.49 min; [M+H]+: 427.05.

### Example 59

### 2-Chloro-N—((R)-1-cyclohexyl-ethyl)-5-(2-methylsulfanyl-pyrimidin-4-ylamino)-benzamide

### 59.1 2-Chloro-5-(2-methylsulfanyl-pyrimidin-4-ylamino)-benzoic acid methyl ester

This compound was prepared using a method analogous to that of Example 1 (intermediate 1.1), 4-chloro-2-methylthiopyrimidine replacing iodobenzene.

LC-MS (A): t_{R}=0.77 min; [M+H]+: 309.85

### 59.2 2-Chloro-5-(2-methylsulfanyl-pyrimidin-4-ylamino)-benzoic acid

This compound was prepared using a method analogous to that of Example 1 (intermediate 1.2), intermediate 59.1 replacing intermediate 1.1.

LC-MS (A): t_{R}=0.46 min; [M+H]+: 296.03.

### 59.3 2-Chloro-N—((R)-1-cyclohexyl-ethyl)-5-(2-methylsulfanyl-pyrimidin-4-ylamino)-benzamide

This compound was prepared using a method analogous to that of Example 1 (intermediate 1.3), intermediate 59.2 replacing intermediate 1.2 and (R)-(−)-1-cyclohexylethylamine replacing 1-aminomethyl-cyclohexanol hydrochloride.

LC-MS (A): t_{R}=0.90 min; [M+H]+: 405.64.

### Example 60

### 2-Chloro-N—((S)-1-cyclohexyl-2-hydroxy-ethyl)-5-(2-methylsulfanyl-pyrimidin-4-ylamino)-benzamide

This compound was prepared using a method analogous to that of Example 1 (intermediate 1.3), intermediate 59.2 replacing intermediate 1.2 and L-cyclohexylglycinol replacing 1-aminomethyl-cyclohexanol hydrochloride.

LC-MS (A): t_{R}=0.80 min; [M+H]+: 421.56.

### Example 61

### 2-Chloro-N-(1-hydroxy-cycloheptylmethyl)-5-(2-methylsulfanyl-pyrimidin-4-ylamino)-benzamide

This compound was prepared using a method analogous to that of Example 1 (intermediate 1.3), intermediate 59.2 replacing intermediate 1.2 and intermediate 45.2 replacing 1-aminomethyl-cyclohexanol hydrochloride.

LC-MS (B): t_{R}=0.59 min; [M+H]+: 421.09.

### Example 62

### 2-Chloro-5-[(2,6-dichloro-pyrimidin-4-yl)-methyl-amino]-N-(1-hydroxy-cyclohexylmethyl)-benzamide

### 62.1 2-Chloro-5-(2,6-dichloro-pyrimidin-4-ylamino)-benzoic acid methyl ester

This compound was prepared using a method analogous to that of Example 1 (intermediate 1.1), 2,4,6-trichloropyrimidine replacing iodobenzene.

LC-MS (B): t_{R}=0.86 min; [M+H]+: 333.99

### 62.2 2-Chloro-5-[(2,6-dichloro-pyrimidin-4-yl)-methyl-amino]-benzoic acid methyl ester

This compound was prepared using a method analogous to that of Example 11 (intermediate 11.1), intermediate 62.1 replacing intermediate 10.1.

LC-MS (B): t_{R}=0.89 min; [M+H]+: 345.96

### 62.3 2-Chloro-5-[(2,6-dichloro-pyrimidin-4-yl)-methyl-amino]-benzoic acid

This compound was prepared using a method analogous to that of Example 1 (intermediate 1.2), intermediate 62.2 replacing intermediate 1.1. This compound was contaminated with 2-chloro-5-[(6-chloro-2-methoxy-pyrimidin-4-yl)-methyl-amino]-benzoic acid.

LC-MS (B): t_{R}=0.75 min; [M+H]+: 331.95

### 62.4 2-Chloro-5-[(2,6-dichloro-pyrimidin-4-yl)-methyl-amino]-N-(1-hydroxy-cyclohexylmethyl)-benzamide

This compound was prepared using a method analogous to that of Example 1 (intermediate 1.3), intermediate 62.3 replacing intermediate 1.2.

LC-MS (B): t_{R}=0.79 min; [M+H]+: 443.12

### Example 63

### 2-Chloro-5-[(6-chloro-2-methoxy-pyrimidin-4-yl)-methyl-amino]-N-(1-hydroxy-cyclohexylmethyl)-benzamide

This compound was isolated as a by-product in the preparation of intermediate 62.4.

LC-MS (B): t_{R}=0.76 min; [M+H]+: 439.18

### Example 64

### 2-Chloro-5-[(6-chloro-2-oxo-1,2-dihydro-pyrimidin-4-yl)-methyl-amino]-N-(1-hydroxy-cyclohexylmethyl)-benzamide

This compound was prepared using a method analogous to that of Example 16 (intermediate 16.1), intermediate 62.4 replacing intermediate 10.2 except that it was purified by preparative LC-MS using method V.

LC-MS (B): t_{R}=0.55 min; [M+H]+: 425.11

### Example 65

### 2-Chloro-N-(1-hydroxy-cyclohexylmethyl)-5-[methyl-(6-methylsulfanyl-pyrimidin-4-yl)-amino]-benzamide

### 65.1 Methyl 2-chloro-5-((6-methylsulfanylpyrimidin-4-yl)amino)benzoate

This compound was prepared using a method analogous to that of Example 1 (intermediate 1.1), 2,4,6-trichloropyrimidine replacing iodobenzene.

LC-MS (A): t_{R}=0.87 min; [M+H]+: 309.96

### 65.2 Methyl 2-chloro-5-(methyl(6-methylsulfanylpyrimidin-4-yl)amino)benzoate

This compound was prepared using a method analogous to that of Example 11 (intermediate 11.1), intermediate 65.1 replacing intermediate 10.1

LC-MS (B): t_{R}=0.68 min; [M+H]+: 324.14

### 65.3 2-Chloro-5-(methyl(6-methylsulfanylpyrimidin-4-yl)amino)benzoic acid

This compound was prepared using a method analogous to that of Example 1 (intermediate 1.2), intermediate 65.2 replacing intermediate 1.1.

LC-MS (B): t_{R}=0.51 min; [M+H]+: 310.02

### 65.4 2-Chloro-N-(1-hydroxy-cyclohexylmethyl)-5-[methyl-(6-methylsulfanyl-pyrimidin-4-yl)-amino]-benzamide

This compound was prepared using a method analogous to that of Example 1 (intermediate 1.3), intermediate 65.3 replacing intermediate 1.2.

LC-MS (B): t_{R}=0.60 min; [M+H]+: 421.15

### Example 66

### 2-Chloro-N-(1-hydroxy-cyclohexylmethyl)-5-[(6-methanesulfonyl-pyrimidin-4-yl)-methyl-amino]-benzamide

To a solution of intermediate 65.4 (830 mg) in DCM (10 mL) was added 3-chloroperbenzoic acid (1021 mg) at 0° C. The reaction mixture was stirred for 1 h at 0° C. then allowed to warm up ON to RT. The DCM was evaporated off and the residue taken up in EtOAc. The organic phase was washed with a 10% solution of Na2CO3, dried over MgSO₄ and concentrated in vacuo. The crude was purified by CC (Hept/EtOAc 1/1 to 0/1) to give 231 mg of the titled compound as a white foam.

LC-MS (B): t_{R}=0.62 min; [M+H]+: 453.10

### Example 67

### 2-Chloro-N-(1-hydroxy-cyclohexylmethyl)-5-[(6-methoxy-pyrimidin-4-yl)-methyl-amino]-benzamide

Example 66 (48 mg) was mixed with a 5.4 M solution of NaOMe in MeOH (0.196 mL) and the reaction mixture was stirred ON at RT. It was quenched with H₂O and extracted with EtOAc. The organic phase was washed with a sat. solution of NaHCO₃ and brine, dried over MgSO₄ and concentrated in vacuo. The crude material was purified by CC (Hept/EtOAc 1/1 to 0/1) to give 32 mg of the titled compound as a white foam.

LC-MS (B): t_{R}=0.60 min; [M+H]+: 405.20

### Example 68

### 2-Chloro-N-(1-hydroxy-cyclohexylmethyl)-5-[methyl-(6-oxo-1,6-dihydro-pyrimidin-4-yl)-amino]-benzamide

This compound was prepared using a method analogous to that of Example 16 (intermediate 16.1), example 66 replacing intermediate 10.2 except that it was purified by preparative LC-MS using method V.

LC-MS (B): t_{R}=0.53 min; [M+H]+: 391.18

### Example 69

### 2-Chloro-N-(1-hydroxy-cyclohexylmethyl)-5-(pyrimidin-2-ylamino)-benzamide

### 69.1 2-Chloro-5-(pyrimidin-2-ylamino)-benzoic acid methyl ester

This compound was prepared using a method analogous to that of Example 1 (intermediate 1.1), 2-chloropyrimidine replacing iodobenzene.

LC-MS (A): t_{R}=0.93 min; [M+H]+: 265.82

### 69.2 2-Chloro-5-(pyrimidin-2-ylamino)-benzoic acid

This compound was prepared using a method analogous to that of Example 1 (intermediate 1.2), intermediate 69.1 replacing intermediate 1.1.

LC-MS (A): t_{R}=0.80 min; [M+H]+: 250.54

### 69.3 2-Chloro-N-(1-hydroxy-cyclohexylmethyl)-5-(pyrimidin-2-ylamino)-benzamide

This compound was prepared using a method analogous to that of Example 1 (intermediate 1.3), intermediate 69.2 replacing intermediate 1.2.

LC-MS (A): t_{R}=0.85 min; [M+H]+: 361.39

### Example 70

### 2-Chloro-N-(1-hydroxy-cyclohexyl methyl)-5-(methyl-pyrimidin-2-yl-amino)-benzamide

This compound was prepared using a method analogous to that of Example 11 (intermediate 11.1), intermediate 69.3 replacing intermediate 10.1.

LC-MS (A): t_{R}=0.83 min; [M+H]+: 375.70

### Example 71

### 2-Chloro-N-(1-hydroxy-cyclohexylmethyl)-5-[(2-methoxy-ethyl)-pyrimidin-2-yl-amino]-benzamide

This compound was prepared using a method analogous to that of Example 11 (intermediate 11.1), intermediate 69.3 replacing intermediate 10.1 and 2-bromoethyl methyl ether replacing methyl iodide.

LC-MS (A): t_{R}=0.84 min; [M+H]+: 419.54

### Example 72

### 2-Chloro-N-(1-hydroxy-cyclohexylmethyl)-5-(4-methylsulfanyl-pyrimidin-2-ylamino)-benzamide

### 72.1 2-Chloro-5-(4-methylsulfanyl-pyrimidin-2-ylamino)-benzoic acid methyl ester

This compound was prepared using a method analogous to that of Example 1 (intermediate 1.1), 2-chloro-4-methylsulfanylpyrimidine replacing iodobenzene.

LC-MS (A): t_{R}=1.00 min; [M+H]+: 310.60

### 72.2 2-Chloro-5-(4-methylsulfanyl-pyrimidin-2-ylamino)-benzoic acid

This compound was prepared using a method analogous to that of Example 1 (intermediate 1.2), intermediate 72.1 replacing intermediate 1.1.

LC-MS (A): t_{R}=0.86 min; [M+H]+: 296.36

### 72.3 2-Chloro-N-(1-hydroxy-cyclohexylmethyl)-5-(4-methylsulfanyl-pyrimidin-2-ylamino)-benzamide

This compound was prepared using a method analogous to that of Example 1 (intermediate 1.3), intermediate 72.2 replacing intermediate 1.2.

LC-MS (B): t_{R}=0.68 min; [M+H]+: 407.10

### Example 73

### 2-Chloro-N-(1-hydroxy-cyclohexylmethyl)-5-[methyl-(4-methylsulfanyl-pyrimidin-2-yl)-amino]-benzamide

This compound was prepared using a method analogous to that of Example 11 (intermediate 11.1), intermediate 72.3 replacing intermediate 10.1.

LC-MS (B): t_{R}=0.66 min; [M+H]+: 421.14

### Example 74

### 2-Chloro-N-(1-hydroxy-cyclohexylmethyl)-5-[(4-methoxy-pyrimidin-2-yl)-methyl-amino]-benzamide

### 74.1 2-Chloro-N-(1-hydroxy-cyclohexylmethyl)-5-[methyl-(4-methanesulfonyl-pyrimidin-2-yl)-amino]-benzamide

This compound was prepared using a method analogous to that of Example 66, example 73 replacing intermediate 65.4.

LC-MS (B): t_{R}=0.66 min; [M+H]+: 453.13

### 74.2 2-Chloro-N-(1-hydroxy-cyclohexylmethyl)-5-[(4-methoxy-pyrimidin-2-yl)-methyl-amino]-benzamide

This compound was prepared using a method analogous to that of Example 67, intermediate 74.1 replacing example 66.

LC-MS (B): t_{R}=0.55 min; [M+H]+: 405.18

### Example 75

### 2-Chloro-N-(1-hydroxy-cyclohexylmethyl)-5-[methyl-(6-oxo-1,6-dihydro-pyrimidin-2-yl)-amino]-benzamide

This compound was prepared using a method analogous to that of Example 16 (intermediate 16.1), intermediate 74.1 replacing intermediate 10.2 except that the reaction mixture was stirred for 1 h at 45° C. and that the crude was purified by preparative LC-MS using method V.

LC-MS (B): t_{R}=0.52 min; [M+H]+: 391.20

### Example 76

### 2-Chloro-N-(1-hydroxy-cyclohexylmethyl)-5-(pyrimidin-5-ylamino)-benzamide

### 76.1 2-Chloro-5-(pyrimidin-5-ylamino)-benzoic acid methyl ester

This compound was prepared using a method analogous to that of Example 1 (intermediate 1.1), 5-bromopyrimidine replacing iodobenzene.

LC-MS (A): t_{R}=0.92 min; [M+H]+: 265.17

### 76.2 2-Chloro-5-(pyrimidin-5-ylamino)-benzoic acid

This compound was prepared using a method analogous to that of Example 1 (intermediate 1.2), intermediate 76.1 replacing intermediate 1.1.

LC-MS (A): t_{R}=0.78 min; [M+H]+: 250.76

### 76.3 2-Chloro-N-(1-hydroxy-cyclohexylmethyl)-5-(pyrimidin-5-ylamino)-benzamide

This compound was prepared using a method analogous to that of Example 1 (intermediate 1.3), intermediate 76.2 replacing intermediate 1.2.

LC-MS (A): t_{R}=0.83 min; [M+H]+: 361.45

### Example 77

### 2-Chloro-N-(1-hydroxy-cyclohexylmethyl)-5-[(2-methoxy-ethyl)-pyrimidin-5-yl-amino]-benzamide

This compound was prepared using a method analogous to that of Example 11 (intermediate 11.1), intermediate 76.3 replacing intermediate 10.1 and 2-bromoethyl methyl ether replacing methyl iodide.

LC-MS (A): t_{R}=0.84 min; [M+H]+: 418.84

### Example 78

### 2-Chloro-N-(1-hydroxy-cyclohexylmethyl)-5-(pyrazin-2-ylamino)-benzamide

### 78.1 2-Chloro-5-(pyrazin-2-ylamino)-benzoic acid methyl ester

This compound was prepared using a method analogous to that of Example 1 (intermediate 1.1), 2-chloropyrazine replacing iodobenzene.

LC-MS (A): t_{R}=0.93 min; [M+H]+: 264.47

### 78.2 2-Chloro-5-(pyrazin-2-ylamino)-benzoic acid

This compound was prepared using a method analogous to that of Example 1 (intermediate 1.2), intermediate 78.1 replacing intermediate 1.1.

LC-MS (A): t_{R}=0.81 min; [M+H]+: 250.74

### 78.3 2-Chloro-N-(1-hydroxy-cyclohexylmethyl)-5-(pyrazin-2-ylamino)-benzamide

This compound was prepared using a method analogous to that of Example 1 (intermediate 1.3), intermediate 78.2 replacing intermediate 1.2.

LC-MS (A): t_{R}=0.86 min; [M+H]+: 361.53

### Example 79

### 2-Chloro-N-(1-hydroxy-cyclohexylmethyl)-5-(methyl-pyrazin-2-yl-amino)-benzamide

### 79.1 2-Chloro-5-(methyl-pyrazin-2-yl-amino)-benzoic acid methyl ester

This compound was prepared using a method analogous to that of Example 11 (intermediate 11.1), intermediate 78.1 replacing intermediate 10.1.

LC-MS (A): t_{R}=0.97 min; [M+H]+: 279.13

### 79.2 2-Chloro-5-(methyl-pyrazin-2-yl-amino)-benzoic acid

This compound was prepared using a method analogous to that of Example 1 (intermediate 1.2), intermediate 79.1 replacing intermediate 1.1.

LC-MS (A): t_{R}=0.78 min; [M+H]+: 263.95

### 79.3 2-Chloro-N-(1-hydroxy-cyclohexylmethyl)-5-(methyl-pyrazin-2-yl-amino)-benzamide

This compound was prepared using a method analogous to that of Example 1 (intermediate 1.3), intermediate 79.2 replacing intermediate 1.2.

LC-MS (A): t_{R}=0.82 min; [M+H]+: 375.01

### Example 80

### 2-Chloro-N-(1-hydroxy-cyclohexylmethyl)-5-[(2-methoxy-ethyl)-pyrazin-2-yl-amino]-benzamide

This compound was prepared using a method analogous to that of Example 11 (intermediate 11.1), intermediate 78.3 replacing intermediate 10.1 and 2-bromoethyl methyl ether replacing methyl iodide.

LC-MS (A): t_{R}=0.88 min; [M+H]+: 419.66

### Example 81

### 2-Chloro-5-[(6-chloro-pyrazin-2-yl)-methyl-amino]-N-(1-hydroxy-cyclohexylmethyl)-benzamide

### 81.1 2-Chloro-5-(6-chloro-pyrazin-2-ylamino)-benzoic acid methyl ester

This compound was prepared using a method analogous to that of Example 1 (intermediate 1.1), 2,6-dichloropyrazine replacing iodobenzene.

LC-MS (B): t_{R}=0.82 min; [M+CH₃CN+H]+: 339.09

### 81.2 2-Chloro-5-[(6-chloro-pyrazin-2-yl)-methyl-amino]-benzoic acid methyl ester

This compound was prepared using a method analogous to that of Example 11 (intermediate 11.1), intermediate 81.1 replacing intermediate 10.1.

LC-MS (C): t_{R}=0.92 min; [M+CH₃CN+H]+: 353.08

### 81.3 2-Chloro-5-[(6-chloro-pyrazin-2-yl)-methyl-amino]-benzoic acid

This compound was prepared using a method analogous to that of Example 1 (intermediate 1.2), intermediate 81.2 replacing intermediate 1.1.

LC-MS (B): t_{R}=0.71 min; [M+CH₃CN+H]+: 339.08

### 81.4 2-Chloro-5-[(6-chloro-pyrazin-2-yl)-methyl-amino]-N-(1-hydroxy-cyclohexylmethyl)-benzamide

This compound was prepared using a method analogous to that of Example 1 (intermediate 1.3), intermediate 81.3 replacing intermediate 1.2.

LC-MS (B): t_{R}=0.76 min; [M+H]+: 409.13

### Example 82

### 2-Chloro-N-(1-hydroxy-cyclohexylmethyl)-5-[(6-methoxy-pyrazin-2-yl)-methyl-amino]-benzamide

This compound was prepared using a method analogous to that of Example 12, intermediate 81.4 replacing intermediate 10.3 except that the reaction mixture was stirred for 1 h at 40° C. then for 18 h at RT and that the crude was purified by preparative LC-MS using method VI.

LC-MS (B): t_{R}=0.70 min; [M+H]+: 405.19

### Example 83

### 2-Chloro-N-(1-hydroxy-cyclohexylmethyl)-5-[methyl-(6-methylamino-pyrazin-2-yl)-amino]-benzamide

This compound was prepared using a method analogous to that of Example 14, intermediate 81.4 replacing intermediate 11.3 except that the reaction mixture was stirred for 5 days at 100° C. and additional amounts of the 41% aqueous solution of methylamine are necessary for completion of the reaction.

LC-MS (B): t_{R}=0.57 min; [M+H]+: 404.30

### Example 84

### 2-Chloro-5-[(6-dimethylamino-pyrazin-2-yl)-methyl-amino]-N-(1-hydroxy-cyclohexylmethyl)-benzamide

This compound was prepared using a method analogous to that of Example 15, intermediate 81.4 replacing intermediate 11.3 except that the reaction mixture was stirred for 8 days at 100° C. and additional amounts of the 40% aqueous solution of dimethylamine are necessary for completion of the reaction.

LC-MS (B): t_{R}=0.60 min; [M+H]+: 418.02

### Example 85

### 2-Chloro-N-(1-hydroxy-cyclohexylmethyl)-5-[methyl-(6-oxo-1,6-dihydro-pyrazin-2-yl)-amino]-benzamide

To a solution of intermediate 81.4 (40 mg) in dimethylsufoxide (0.090 mL) was added a 32% aqueous solution of NaOH (0.090 mL) at RT and the reaction mixture was stirred for 1 h at 150° C. It was concentrated to dryness and purified by preparative LC-MS using method IV.

LC-MS (B): t_{R}=0.57 min; [M+H]+: 390.97

### Example 86

### 2-Chloro-5-[(3-chloro-pyrazin-2-yl)-methyl-amino]-N-(1-hydroxy-cyclohexylmethyl)-benzamide

### 86.1 2-Chloro-5-(3-chloro-pyrazin-2-ylamino)-benzoic acid methyl ester

This compound was prepared using a method analogous to that of Example 8 (intermediate 8.1), 2-amino-3-chloropyrazine replacing 4-aminopyrimidine.

LC-MS (B): t_{R}=0.81 min; [M+H]+: 298.22

### 86.2 2-Chloro-5-((3-chloropyrazin-2-yl)-methyl-amino)benzoic acid methyl ester

This compound was prepared using a method analogous to that of Example 11 (intermediate 11.1), intermediate 86.1 replacing intermediate 10.1.

LC-MS (B): t_{R}=0.83 min; [M+H]+: 312.09

### 86.3 2-Chloro-5-((3-chloropyrazin-2-yl)-methyl-amino)benzoic acid

This compound was prepared using a method analogous to that of Example 1 (intermediate 1.2), intermediate 86.2 replacing intermediate 1.1.

LC-MS (B): t_{R}=0.67 min; [M+H]+: 298.22

### 86.4 2-Chloro-5-[(3-chloro-pyrazin-2-yl)-methyl-amino]-N-(1-hydroxy-cyclohexylmethyl)-benzamide

This compound was prepared using a method analogous to that of Example 10 (intermediate 10.3), intermediate 86.3 replacing intermediate 10.2

LC-MS (B): t_{R}=0.72 min; [M+H]+: 409.11

### Example 87

### 2-Chloro-5-[(5-chloro-pyrazin-2-yl)-methyl-amino]-N-(1-hydroxy-cyclohexylmethyl)-benzamide

### 87.1 2-Chloro-5-(5-chloro-pyrazin-2-ylamino)-benzoic acid methyl ester

This compound was prepared using a method analogous to that of Example 1 (intermediate 1.1), 2,5-dichloropyrazine replacing iodobenzene.

LC-MS (B): t_{R}=0.84 min; [M+H]+: 298.06

### 87.2 2-Chloro-5-[(5-chloro-pyrazin-2-yl)-methyl-amino]-benzoic acid methyl ester

This compound was prepared using a method analogous to that of Example 11 (intermediate 11.1), intermediate 87.1 replacing intermediate 10.1.

LC-MS (B): t_{R}=0.87 min; [M+H]+: 312.10

### 87.3 2-Chloro-5-[(5-chloro-pyrazin-2-yl)-methyl-amino]-benzoic acid

This compound was prepared using a method analogous to that of Example 1 (intermediate 1.2), intermediate 87.2 replacing intermediate 1.1.

LC-MS (B): t_{R}=0.72 min; [M+H]+: 298.07.

### 87.4 2-Chloro-5-[(5-chloro-pyrazin-2-yl)-methyl-amino]-N-(1-hydroxy-cyclohexylmethyl)-benzamide

This compound was prepared using a method analogous to that of Example 10 (intermediate 10.3), intermediate 87.3 replacing intermediate 10.2

LC-MS (B): t_{R}=0.76 min; [M+H]+: 409.27

### Example 88

### 2-Chloro-N-(1-hydroxy-cyclohexylmethyl)-5-[(5-methoxy-pyrazin-2-yl)-methyl-amino]-benzamide

This compound was prepared using a method analogous to that of Example 12, intermediate 87.4 replacing intermediate 10.3 except that the reaction mixture was stirred for 18 h at 40° C. and that the crude was purified by preparative LC-MS using method VI.

LC-MS (B): t_{R}=0.73 min; [M+H]+: 405.16

### Example 89

### 2-Chloro-5-[(6-chloro-pyridazin-3-yl)-methyl-amino]-N-(1-hydroxy-cyclohexylmethyl)-benzamide

### 89.1 2-Chloro-5-(6-chloro-pyridazin-3-ylamino)-benzoic acid methyl ester

A solution of 3,6-dichloropyridazine (240 mg) and methyl-5-amino-2-chlorobenzoate (299 mg) in EtOH (9 mL) was heated in the microwave for 30 min at 150° C. The reaction was concentrated in vacuo and the crude was purified by CC (Hept/EtOAc 1/0 to 4/6) to give 203 mg of the titled compound as a light yellow solid.

LC-MS (B): t_{R}=0.72 min; [M+H]+: 298.06

### 89.2 2-Chloro-5-[(6-chloro-pyridazin-3-yl)-methyl-amino]-benzoic acid methyl ester

This compound was prepared using a method analogous to that of Example 11 (intermediate 11.1), intermediate 89.1 replacing intermediate 10.1.

LC-MS (B): t_{R}=0.74 min; [M+H]+: 312.09

### 89.3 2-Chloro-5-[(6-chloro-pyridazin-3-yl)-methyl-amino]-benzoic acid

This compound was prepared using a method analogous to that of Example 1 (intermediate 1.2), intermediate 89.2 replacing intermediate 1.1

LC-MS (B): t_{R}=0.59 min; [M+H]+: 298.06.

### 89.4 2-Chloro-5-[(6-chloro-pyridazin-3-yl)-methyl-amino]-N-(1-hydroxy-cyclohexylmethyl)-benzamide

This compound was prepared using a method analogous to that of Example 1 (intermediate 1.3), intermediate 89.3 replacing intermediate 1.2

LC-MS (B): t_{R}=0.66 min; [M+H]+: 409.10

### Example 90

### 2-Chloro-N-(1-hydroxy-cyclohexylmethyl)-5-[methyl-(6-oxo-1,6-dihydro-pyridazin-3-yl)-amino]-benzamide

This compound was prepared using a method analogous to that of Example 16 (intermediate 16.1), intermediate 89.4 replacing intermediate 10.2 except that the reaction mixture was stirred for 5 days at 110° C. and that the crude was purified by preparative LC-MS using method V.

LC-MS (B): t_{R}=0.56 min; [M+H]+: 391.09

### Example 91

### 2-Chloro-N-(1-hydroxy-cyclohexylmethyl)-5-[(6-methoxy-pyridazin-3-yl)-methyl-amino]-benzamide

This compound was prepared using a method analogous to that of Example 12, intermediate 89.4 replacing intermediate 10.3.

LC-MS (B): t_{R}=0.52 min; [M+H]+: 405.16

### Example 92

### 2-Chloro-N-(1-hydroxy-cyclohexylmethyl)-5-(pyridin-2-ylamino)-benzamide

### 92.1 2-Chloro-5-(pyridin-2-ylamino)-benzoic acid methyl ester

This compound was prepared using a method analogous to that of Example 1 (intermediate 1.1), 2-bromopyridine replacing iodobenzene.

LC-MS (A): t_{R}=0.71 min; [M+H]+: 263.05

### 92.2 2-Chloro-5-(pyridin-2-ylamino)-benzoic acid hydrochloride salt

This compound was prepared using a method analogous to that of Example 1 (intermediate 1.2), intermediate 92.1 replacing intermediate 1.1 except that the product precipitated from the aqueous phase after acidification and was isolated as a hydrochloride salt.

LC-MS (A): t_{R}=0.60 min; [M+H]+: 249.27

### 92.3 2-Chloro-N-(1-hydroxy-cyclohexylmethyl)-5-(pyridin-2-ylamino)-benzamide

This compound was prepared using a method analogous to that of Example 1 (intermediate 1.3), intermediate 92.2 replacing intermediate 1.2.

LC-MS (A): t_{R}=0.70 min; [M+H]+: 360.78

### Example 93

### 2-Chloro-N-(1-hydroxy-cyclohexylmethyl)-5-(methyl-pyridin-2-yl-amino)-benzamide

### 93.1 2-Chloro-5-(methyl-pyridin-2-yl-amino)-benzoic acid methyl ester

This compound was prepared using a method analogous to that of Example 11 (intermediate 11.1), intermediate 92.2 replacing intermediate 10.1 except that additional amounts of methyl iodide (2×1 eq) were used for completion of the reaction and the reaction was stirred for 24 h at 40° C.

LC-MS (A): t_{R}=0.67 min; [M+H]+: 276.98

### 93.2 2-Chloro-5-(methyl-pyridin-2-yl-amino)-benzoic acid

This compound was prepared using a method analogous to that of Example 1 (intermediate 1.2), intermediate 93.1 replacing intermediate 1.1 except that a 1M aqueous solution of NaOH was used instead of lithium hydroxide in H₂O.

LC-MS (A): t_{R}=0.57 min; [M+H]+: 262.89

### 93.3 2-Chloro-N-(1-hydroxy-cyclohexylmethyl)-5-(methyl-pyridin-2-yl-amino)-benzamide

This compound was prepared using a method analogous to that of Example 10 (intermediate 10.3), intermediate 93.2 replacing intermediate 10.2

LC-MS (C): t_{R}=0.52 min; [M+H]+: 374.12

### Example 94

### 2-Chloro-N-(1-hydroxy-cyclohexylmethyl)-5-(6-oxo-1,6-dihydro-pyridin-2-ylamino)-benzamide

### 94.1 2-Chloro-6-(2-trimethylsilanyl-ethoxymethoxy)-pyridine

To a suspension of 6-chloro-2-pyridinol (2.00 g) in acetone (77 mL) was added potassium carbonate (7.47 g) and (2-chloromethoxyethyl)-trimethylsilane (3.28 mL) and the mixture was stirred for 1 h at RT. It was filtered off and the filtrate was concentrated in vacuo. The crude was purified by CC (Hept/EtOAc 1/0 to 1/1) to give 2.5 g of the titled compound as a yellowish oil in addition to 1.35 g of the N-protected product as a colorless oil (see intermediate 94.2).

LC-MS (A): t_{R}=1.13 min; [M+H]+: 260.76

### 94.2 6-Chloro-1-(2-trimethylsilanyl-ethoxymethyl)-1H-pyridin-2-one

This compound was isolated as by-product in the preparation of intermediate 94.1

LC-MS (A): t_{R}=1.01 min; [M+H]+: 260.74

### 94.3 2-Chloro-5-[6-(2-trimethylsilanyl-ethoxymethoxy)-pyridin-2-ylamino]-benzoic acid methyl ester

This compound was prepared using a method analogous to that of Example 1 (intermediate 1.1), intermediate 94.1 replacing iodobenzene.

LC-MS (A): t_{R}=1.17 min; [M+H]+: 409.71

### 94.4 2-Chloro-N-(1-hydroxy-cyclohexylmethyl)-5-(6-oxo-1,6-dihydro-pyridin-2-ylamino)-benzamide

This compound was prepared using a method analogous to that of Example 1 (intermediate 1.2), intermediate 94.3 replacing intermediate 1.1 except that a 1M aqueous solution of NaOH was used instead of lithium hydroxide in H₂O.

LC-MS (A): t_{R}=1.08 min; [M+H]+: 395.31

### 94.5 2-Chloro-N-(1-hydroxy-cyclohexylmethyl)-5-[6-(2-trimethylsilanyl-ethoxymethoxy)-pyridin-2-ylamino]-benzamide

This compound was prepared using a method analogous to that of Example 1 (intermediate 1.3), intermediate 94.4 replacing intermediate 1.2.

LC-MS (B): t_{R}=1.00 min; [M+H]+: 506.13

### 94.6 2-Chloro-N-(1-hydroxy-cyclohexylmethyl)-5-(6-oxo-1,6-dihydro-pyridin-2-ylamino)-benzamide

This compound was isolated as by-product in the previous step 94.5 where the cleavage of the silylated protecting group partially occurred.

LC-MS (B): t_{R}=0.56 min; [M+H]+: 376.12

### Example 95

### 2-Chloro-N-(1-hydroxy-cyclohexylmethyl)-5-[methyl-(6-oxo-1,6-dihydro-pyridin-2-yl)-amino]-benzamide

### 95.1 2-Chloro-5-{methyl-[6-(2-trimethylsilanyl-ethoxymethoxy)-pyridin-2-yl]-amino}-benzoic acid methyl ester

This compound was prepared using a method analogous to that of Example 11 (intermediate 11.1), intermediate 94.2 replacing intermediate 10.1 except that the reaction was stirred for 24 h at RT.

LC-MS (B): t_{R}=1.13 min; [M+H]+: 423.12

### 95.2 2-Chloro-5-{methyl-[6-(2-trimethylsilanyl-ethoxymethoxy)-pyridin-2-yl]-amino}-benzoic acid

This compound was prepared using a method analogous to that of Example 1 (intermediate 1.2), intermediate 95.1 replacing intermediate 1.1 except that a 1M aqueous solution of NaOH was used instead of lithium hydroxide in H₂O.

LC-MS (B): t_{R}=1.02 min; [M+H]+: 409.15

### 95.3 2-Chloro-N-(1-hydroxy-cyclohexylmethyl)-5-[methyl-(6-oxo-1,6-dihydro-pyridin-2-yl)-amino]-benzamide

To a solution of intermediate 95.2 (130 mg) and DIPEA (0.218 mL) in DCM (2 mL) was added HOBT (52 mg) and EDC.HCl (73 mg) at RT. The solution was stirred for 5 min at RT and 1-aminomethyl-cyclohexanol hydrochloride (63 mg) was added. The reaction mixture was further stirred for 18 h at 35° C. and diluted with DCM. The organic phase was washed with a sat. solution of NaHCO₃ and brine, dried over MgSO₄ and concentrated in vacuo. The crude material was dissolved in DCM (0.5 mL) and treated with TFA (0.5 mL). The solution was stirred for 30 min at 0° C., quenched with a sat. solution of NaHCO₃ at 0° C. and extracted with DCM. The organic phase was washed with brine, dried over MgSO₄ and concentrated in vacuo. The crude was purified by CC (EtOAc/MeOH 1/0 to 7/3) to give the titled compound as a white solid

LC-MS (B): t_{R}=0.56 min; [M+H]+: 390.16

### Example 96

### 2-Chloro-N-(1-hydroxy-cyclohexylmethyl)-5-(1-methyl-6-oxo-1,6-dihydro-pyridin-2-ylamino)-benzamide

### 96.1 6-Chloro-1-methyl-1H-pyridin-2-one

To a suspension of 6-chloro-2-pyridinol (3 g) in acetone (116 mL) was added potassium carbonate (11.2 g) and methyl iodide (4.92 mL) and the mixture was stirred for 5 h at RT. It was filtered off and the filtrate was concentrated in vacuo. The crude was purified by CC (Hept/EtOAc 8/2 to 0/1) to give 1.96 g of the titled compound as a white solid.

LC-MS (B): t_{R}=0.39 min; [M+H]+: 144.14

### 96.2 2-Chloro-5-(1-methyl-6-oxo-1,6-dihydro-pyridin-2-ylamino)-benzoic acid methyl ester

This compound was prepared using a method analogous to that of Example 1 (intermediate 1.1), intermediate 96.1 replacing iodobenzene.

LC-MS (A): t_{R}=0.61 min; [M+H]+: 293.23

### 96.3 2-Chloro-5-(1-methyl-6-oxo-1,6-dihydro-pyridin-2-ylamino)-benzoic acid

This compound was prepared using a method analogous to that of Example 1 (intermediate 1.2), intermediate 96.2 replacing intermediate 1.1 except that a 1M aqueous solution of NaOH was used instead of lithium hydroxide in H₂O.

LC-MS (B): t_{R}=0.49 min; [M+H]+: 279.20

### 96.4 2-Chloro-N-(1-hydroxy-cyclohexylmethyl)-5-(1-methyl-6-oxo-1,6-dihydro-pyridin-2-ylamino)-benzamide

This compound was prepared using a method analogous to that of Example 1 (intermediate 1.3), intermediate 96.3 replacing intermediate 1.2 except that the compound was further purified by preparative LC-MS using method IV.

LC-MS (B): t_{R}=0.58 min; [M+H]+: 389.93

### Example 97

### 2-Chloro-N-(1-hydroxy-cyclohexylmethyl)-5-[1-(2-methoxy-ethyl)-6-oxo-1,6-dihydro-pyridin-2-ylamino]-benzamide

### 97.1 6-Chloro-1-(2-methoxy-ethyl)-1H-pyridin-2-one

This compound was prepared using a method analogous to that of Example 96 (intermediate 96.1), 2-bromoethyl methyl ether replacing methyl iodide except that the reaction mixture was stirred for 17 h at 70° C.

LC-MS (A): t_{R}=0.89 min; [M+H]+: 188.52

### 97.2 2-Chloro-5-[1-(2-methoxy-ethyl)-6-oxo-1,6-dihydro-pyridin-2-ylamino]-benzoic acid methyl ester

This compound was prepared using a method analogous to that of Example 1 (intermediate 1.1), intermediate 97.1 replacing iodobenzene.

LC-MS (A): t_{R}=1.04 min; [M+H]+: 337.30

### 97.3 2-Chloro-5-[1-(2-methoxy-ethyl)-6-oxo-1,6-dihydro-pyridin-2-ylamino]-benzoic acid

This compound was prepared using a method analogous to that of Example 1 (intermediate 1.2), intermediate 97.2 replacing intermediate 1.1 except that a 1M aqueous solution of NaOH was used instead of lithium hydroxide in H₂O.

LC-MS (A): t_{R}=0.92 min; [M+H]+: 323.32

### 97.4 2-Chloro-N-(1-hydroxy-cyclohexylmethyl)-5-[1-(2-methoxy-ethyl)-6-oxo-1,6-dihydro-pyridin-2-ylamino]-benzamide

This compound was prepared using a method analogous to that of Example 1 (intermediate 1.3), intermediate 97.3 replacing intermediate 1.2.

LC-MS (A): t_{R}=0.96 min; [M+H]+: 433.78

### Example 98

### 2-Chloro-N-(1-hydroxy-cyclohexyl methyl)-5-{[1-(2-methoxy-ethyl)-6-oxo-1,6-dihydro-pyridin-2-yl]-methyl-amino}-benzamide

This compound was prepared using a method analogous to that of Example 11 (intermediate 11.1), intermediate 97.3 replacing intermediate 10.1 except that the reaction was stirred for 17 h at RT.

LC-MS (C): t_{R}=1.11 min; [M+H]+: 447.83

### Example 99

### 2-Chloro-N-(1-hydroxy-cyclohexylmethyl)-5-(1-methyl-2-oxo-1,2-dihydro-pyridin-3-ylamino)-benzamide

### 99.1 3-Bromo-1-methyl-1H-pyridin-2-one

This compound was prepared using a method analogous to that of Example 96 (intermediate 96.1), 3-bromo-2-hydroxypyridine replacing 6-chloropyridinol except that the reaction mixture was stirred for 18 h at RT.

LC-MS (B): t_{R}=0.34 min; [M+H]+: 188.10

### 99.2 2-Chloro-5-(1-methyl-2-oxo-1,2-dihydro-pyridin-3-ylamino)-benzoic acid methyl ester

This compound was prepared using a method analogous to that of Example 1 (intermediate 1.1), intermediate 99.1 replacing iodobenzene except that the reaction mixture was stirred for 6 days at 80° C.

LC-MS (B): t_{R}=0.68 min; [M+H]+: 293.12

### 99.3 2-Chloro-5-(1-methyl-2-oxo-1,2-dihydro-pyridin-3-ylamino)-benzoic acid

This compound was prepared using a method analogous to that of Example 1 (intermediate 1.2), intermediate 99.2 replacing intermediate 1.1 except that it precipitated out the aqueous phase after acidification and was filtered.

LC-MS (B): t_{R}=0.55 min; [M+H]+: 279.09

### 99.4 2-Chloro-N-(1-hydroxy-cyclohexylmethyl)-5-(1-methyl-2-oxo-1,2-dihydro-pyridin-3-ylamino)-benzamide

This compound was prepared using a method analogous to that of Example 1 (intermediate 1.3), intermediate 99.3 replacing intermediate 1.2.

LC-MS (B): t_{R}=0.62 min; [M+H]+: 390.11

### Example 100

### 2-Chloro-N-(1-hydroxy-cyclohexyl methyl)-5-(2-methyl-2H-pyrazol-3-ylamino)-benzamide

### 100.1 2-Chloro-5-(2-methyl-2H-pyrazol-3-ylamino)-benzoic acid methyl ester

This compound was prepared using a method analogous to that of Example 8 (intermediate 8.1), 2-methyl-2H-pyrazol-3-ylamine replacing 4-aminopyrimidine except that the reaction mixture was stirred for 2 days at 80° C.

LC-MS (B): t_{R}=0.64 min; [M+H]+: 266.02

(ELN163-0329)

### 100.2 2-Chloro-5-(2-methyl-2H-pyrazol-3-ylamino)-benzoic acid

This compound was prepared using a method analogous to that of Example 1 (intermediate 1.2), intermediate 100.1 replacing intermediate 1.1.

LC-MS (B): t_{R}=0.50 min; [M+H]+: 251.96

### 100.3 2-Chloro-N-(1-hydroxy-cyclohexylmethyl)-5-(2-methyl-2H-pyrazol-3-ylamino)-benzamide

This compound was prepared using a method analogous to that of Example 1 (intermediate 1.3), intermediate 100.2 replacing intermediate 1.2.

LC-MS (B): t_{R}=0.59 min; [M+H]+: 363.10

### Example 101

### 2-Chloro-N-(1-hydroxy-cyclohexylmethyl)-5-[methyl-(2-methyl-2H-pyrazol-3-yl)-amino]-benzamide

This compound was prepared using a method analogous to that of Example 11 (intermediate 11.1), intermediate 100.3 replacing intermediate 10.1 except that the reaction was stirred for 18 h at RT.

LC-MS (B): t_{R}=0.66 min; [M+H]+: 377.11

### Example 102

### 2-Chloro-5-(6-fluoro-pyridin-2-yloxy)-N-(1-hydroxy-cyclohexylmethyl)-benzamide

### 102.1 2-chloro-5-((6-fluoropyridin-2-yl)oxy)benzoic acid

To a solution of 2-chloro-5-hydroxybenzoic acid (282 mg) in anh. DMSO (3.3 mL) was added Cs₂CO₃ (1600 mg) and the suspension was stirred for 20 min at RT. 2,6-difluoropyridine (0.150 mL) was added and the reaction mixture was stirred for 18 h at 80° C. It was quenched with H₂O and extracted with EtOAc. The aqueous phase was acidified with a 25% aqueous solution of hydrochloric acid and extracted with EtOAc. The organic phase was dried over MgSO₄ and concentrated in vacuo to give 438 mg of the titled crude compound as a beige solid.

LC-MS (B): t_{R}=0.70 min; [M+H]+: 267.94

### 102.2 2-Chloro-5-(6-fluoro-pyridin-2-yloxy)-N-(1-hydroxy-cyclohexylmethyl)-benzamide

This compound was prepared using a method analogous to that of Example 10 (intermediate 10.3), intermediate 102.1 replacing intermediate 10.2

LC-MS (B): t_{R}=0.75 min; [M+H]+: 379.06

### Example 103

### 2-Chloro-5-(4-fluoro-pyridin-2-yloxy)-N-(1-hydroxy-cyclohexylmethyl)-benzamide

### 103.1 2-chloro-5-((4-fluoropyridin-2-yl)oxy)benzoic acid

This compound was prepared using a method analogous to that of Example 102 (intermediate 102.1), 2,4-difluoropyridine replacing 2,6-difluoropyridine.

LC-MS (B): t_{R}=0.65 min; [M+H]+: 268.01

### 103.2 2-Chloro-5-(4-fluoro-pyridin-2-yloxy)-N-(1-hydroxy-cyclohexylmethyl)-benzamide

This compound was prepared using a method analogous to that of Example 10 (intermediate 10.3), intermediate 103.1 replacing intermediate 10.2

LC-MS (B): t_{R}=0.71 min; [M+H]+: 379.08

### Example 104

### 2-Chloro-5-(2-chloro-pyridin-4-yloxy)-N-(1-hydroxy-cyclohexylmethyl)-benzamide

### 104.1 2-chloro-5-((2-chloropyridin-4-yl)oxy)benzoic acid

This compound was prepared using a method analogous to that of Example 102 (intermediate 102.1), 2-chloro-4-fluoropyridine replacing 2,6-difluoropyridine.

LC-MS (B): t_{R}=0.68 min; [M+H]+: 284.02

### 104.2 2-Chloro-5-(2-chloro-pyridin-4-yloxy)-N-(1-hydroxy-cyclohexylmethyl)-benzamide

This compound was prepared using a method analogous to that of Example 10 (intermediate 10.3), intermediate 104.1 replacing intermediate 10.2

LC-MS (C): t_{R}=0.78 min; [M+H]+: 395.02

### Example 105

### 2-Chloro-N-(1-hydroxy-cyclohexylmethyl)-5-(pyridin-2-yloxy)-benzamide

### 105.1 2-chloro-5-(pyridin-2-yloxy)benzoic acid

This compound was prepared using a method analogous to that of Example 102 (intermediate 102.1), 2-chloropyridine replacing 2,6-difluoropyridine except that the reaction mixture was stirred for 4 days at 120° C.

LC-MS (B): t_{R}=0.64 min; [M+H]+: 249.93

### 105.2 2-Chloro-N-(1-hydroxy-cyclohexylmethyl)-5-(pyridin-2-yloxy)-benzamide

This compound was prepared using a method analogous to that of Example 1 (intermediate 1.3), intermediate 105.1 replacing intermediate 1.2

LC-MS (B): t_{R}=0.69 min; [M+H]+: 361.06

### Example 106

### 2-Chloro-N-(1-hydroxy-cyclohexylmethyl)-5-(pyridin-4-yloxy)-benzamide

### 106.1 Sodium 2-chloro-5-(pyridin-4-yloxy)benzoate

A microwave vial was charged with copper (I) bromide (23 mg), Cs₂CO₃ (2055 mg), 4-hydroxypyridine (300 mg) and methyl-2-chloro-5-iodobenzoate (1122 mg) and flushed with argon. DMSO (4.7 mL) was added followed by 2-pyridyl acetone (0.043 mL) and the reaction mixture was heated to 100° C. for 3 h in the microwave. It was diluted with EtOAc, filtered and the filtrate was washed with H₂O. The aqueous phase was basified with a 1M solution of NaOH and extracted with EtOAc. The crude was purified by CC(RP C18, H₂O/CH₃CN 1/0 to 8/2) to give 1.2 g of the titled compound as a white powder.

LC-MS (B): t_{R}=0.34 min; [M+H]+: 249.98

### 106.2 2-Chloro-N-(1-hydroxy-cyclohexylmethyl)-5-(pyridin-4-yloxy)-benzamide

This compound was prepared using a method analogous to that of Example 10 (intermediate 10.3), intermediate 106.1 replacing intermediate 10.2

LC-MS (B): t_{R}=0.49 min; [M+H]+: 361.26

### Example 107

### 2-Chloro-N-(1-hydroxy-cyclohexylmethyl)-5-(pyridin-3-yloxy)-benzamide

### 107.1 2-chloro-5-(pyridin-3-yloxy)benzoic acid methyl ester

A microwave vial was charged with copper (I) bromide (7.7 mg), Cs₂CO₃ (685 mg), 3-hydroxypyridine (100 mg) and methyl-2-chloro-5-iodobenzoate (374 mg) and flushed with argon. DMSO (1.6 mL) was added followed by 2-pyridyl acetone (0.014 mL) and the reaction mixture was heated to 100° C. for 3 h in the microwave. It was diluted with EtOAc, filtered and the filtrate was washed with H₂O. The organic phase was dried over MgSO₄ and concentrated in vacuo. The crude was purified by CC (Hept/EtOAc 1/0 to 1/1) to give 58 mg of the titled compound as a yellowish waxy solid.

LC-MS (B): t_{R}=0.59 min; [M+H]+: 264.26

### 107.2 2-Chloro-5-(pyridin-3-yloxy)-benzoic acid

This compound was prepared using a method analogous to that of Example 1 (intermediate 1.2), intermediate 107.1 replacing intermediate 1.1 except that a 2M solution of NaOH was used instead of LiOH in H₂O.

LC-MS (B): t_{R}=0.43 min; [M+H]+: 249.94

### 107.3 2-Chloro-N-(1-hydroxy-cyclohexylmethyl)-5-(pyridin-3-yloxy)-benzamide

This compound was prepared using a method analogous to that of Example 10 (intermediate 10.3), intermediate 107.2 replacing intermediate 10.2

LC-MS (B): t_{R}=0.53 min; [M+H]+: 361.14

### Example 108

### 2-Chloro-5-(6-chloro-pyridin-2-yloxy)-N-(1-hydroxy-cyclohexylmethyl)-benzamide

### 108.1 2-Chloro-5-(6-chloro-pyridin-2-yloxy)-benzoic acid

This compound was prepared using a method analogous to that of Example 102 (intermediate 102.1), 2,6-dichloropyridine replacing 2,6-difluoropyridine.

LC-MS (B): t_{R}=0.74 min; [M+H]+: 284.02

### 108.2 2-Chloro-5-(6-chloro-pyridin-2-yloxy)-N-(1-hydroxy-cyclohexylmethyl)-benzamide

This compound was prepared using a method analogous to that of Example 1 (intermediate 1.3), intermediate 108.1 replacing intermediate 1.2.

LC-MS (B): t_{R}=0.78 min; [M+H]+: 394.97

### Example 109

### 2-Chloro-N-(1-hydroxy-cyclohexylmethyl)-5-(6-methylamino-pyridin-2-yloxy)-benzamide

This compound was prepared using a method analogous to that of Example 14, intermediate 108.2 replacing intermediate 11.3 except that the reaction was stirred for 5 days at 100° C. and additional amounts of the 41% solution of methylamine in H₂O (10 eq) were necessary for the completion of the reaction.

LC-MS (B): t_{R}=0.68 min; [M+H]+: 390.11

### Example 110

### 2-Chloro-5-(6-dimethylamino-pyridin-2-yloxy)-N-(1-hydroxy-cyclohexylmethyl)-benzamide

This compound was prepared using a method analogous to that of Example 15, intermediate 108.2 replacing intermediate 11.3 except that the reaction was stirred for 5 days at 100° C. and additional amounts of the 40% solution of dimethylamine in H₂O (5 eq) were necessary for the completion of the reaction.

LC-MS (B): t_{R}=0.82 min; [M+H]+: 404.13

### Example 111

### 2-Chloro-N-(1-hydroxy-cyclohexylmethyl)-5-(6-methoxy-pyridin-2-yloxy)-benzamide

### 111.1 2-Chloro-5-hydroxy-N-(1-hydroxy-cyclohexylmethyl)-benzamide

This compound was prepared using a method analogous to that of Example 1 (intermediate 1.3), 2-chloro-5-hydroxybenzoic acid replacing intermediate 1.2.

LC-MS (B): t_{R}=0.56 min; [M+H]+: 284.18

### 111.2 2-Chloro-N-(1-hydroxy-cyclohexylmethyl)-5-(6-methoxy-pyridin-2-yloxy)-benzamide

This compound was prepared using a method analogous to that of Example 102 (intermediate 102.1), 2-chloro-6-methoxypyridine replacing 2,6-difluoropyridine and intermediate 111.1 replacing 2-chloro-5-hydroxybenzoic acid except that the reaction mixture was stirred for 7 days at 100° C.

LC-MS (B): t_{R}=0.79 min; [M+H]+: 391.13

### Example 112

### 2-Chloro-N-(1-hydroxy-cyclohexylmethyl)-5-(6-oxo-1,6-dihydro-pyridin-2-yloxy)-benzamide

### 112.1 2-chloro-5-((6-oxo-1-((2-(trimethylsilyl)ethoxy)methyl)-1,6-dihydropyridin-2-yl)oxy)benzoic acid

This compound was prepared using a method analogous to that of Example 102 (intermediate 102.1), intermediate 94.2 replacing 2,6-difluoropyridine except that the reaction mixture was stirred for 4 days at 80° C.

LC-MS (B): t_{R}=0.80 min; [M+H]+: 396.22

### 112.2 2-Chloro-N-(1-hydroxy-cyclohexylmethyl)-5-[6-oxo-1-(2-trimethylsilanyl-ethoxymethyl)-1,6-dihydro-pyridin-2-yloxy]-benzamide

This compound was prepared using a method analogous to that of Example 1 (intermediate 1.3), intermediate 112.1 replacing intermediate 1.2.

LC-MS (B): t_{R}=0.85 min; [M+H]+: 507.29

### 112.3 2-Chloro-N-(1-hydroxy-cyclohexylmethyl)-5-(6-oxo-1,6-dihydro-pyridin-2-yloxy)-benzamide

To a solution of intermediate 112.2 (68 mg) in THF (1.18 mL) was added a 1M solution of tetrabutyl ammonium fluoride (0.496 mL) and the reaction mixture was stirred for 2 days at 60° C. Two additional amounts of a 1M solution of tetrabutyl ammonium fluoride (2×0.5 mL) were necessary for the completion of the reaction. The reaction mixture was diluted with DCM and washed with a sat. solution of NaHCO₃. The organic phase was dried over MgSO₄ and concentrated in vacuo. The crude was purified by CC (Hept/EtOAc 1/1 to 0/1) then by preparative LC-MS using method IV to give the titled compound as a white powder.

LC-MS (B): t_{R}=0.61 min; [M+H]+: 377.30

### Example 113

### 2-Chloro-N-(1-hydroxy-cyclohexylmethyl)-5-(1-methyl-6-oxo-1,6-dihydro-pyridin-2-yloxy)-benzamide

### 113.1 2-Chloro-5-(1-methyl-6-oxo-1,6-dihydro-pyridin-2-yloxy)-benzoic acid

This compound was prepared using a method analogous to that of Example 102 (intermediate 102.1), intermediate 96.1 replacing 2,6-difluoropyridine except that the reaction mixture was stirred for 2 days at 85° C.

LC-MS (B): t_{R}=0.52 min; [M+H]+: 280.30

### 113.2 2-Chloro-N-(1-hydroxy-cyclohexylmethyl)-5-(1-methyl-6-oxo-1,6-dihydro-pyridin-2-yloxy)-benzamide

This compound was prepared using a method analogous to that of Example 1 (intermediate 1.3), intermediate 113.1 replacing intermediate 1.2.

LC-MS (B): t_{R}=0.59 min; [M+H]+: 391.25

### Example 114

### 2-Chloro-N-(1-hydroxy-cyclohexylmethyl)-5-(pyrimidin-4-yloxy)-benzamide

### 114.1 2-Chloro-5-(pyrimidin-4-yloxy)-benzoic acid

This compound was prepared using a method analogous to that of Example 102 (intermediate 102.1), 4-chloropyrimidine dihydrochloride replacing 2,6-difluoropyridine except that the reaction mixture was stirred for 3 days at 80° C.

LC-MS (B): t_{R}=0.51 min; [M+H]+: 251.24

### 114.2 2-Chloro-N-(1-hydroxy-cyclohexylmethyl)-5-(pyrimidin-4-yloxy)-benzamide

This compound was prepared using a method analogous to that of Example 1 (intermediate 1.3), intermediate 114.1 replacing intermediate 1.2.

LC-MS (B): t_{R}=0.58 min; [M+H]+: 362.24

### Example 115

### 2-Chloro-5-(2-chloro-pyrimidin-4-yloxy)-N-(1-hydroxy-cyclohexylmethyl)-benzamide

### 115.1 2-chloro-5-((2-chloropyrimidin-4-yl)oxy)benzoic acid

This compound was prepared using a method analogous to that of Example 102 (intermediate 102.1), 2,4-dichloropyrimidine replacing 2,6-difluoropyridine except that the product precipitated out off the aqueous phase after acidification and was isolated by filtration.

LC-MS (B): t_{R}=0.63 min; [M+H]+: 285.16

### 115.2 2-Chloro-5-(2-chloro-pyrimidin-4-yloxy)-N-(1-hydroxy-cyclohexylmethyl)-benzamide

This compound was prepared using a method analogous to that of Example 10 (intermediate 10.3), intermediate 115.1 replacing intermediate 10.2

LC-MS (B): t_{R}=0.69 min; [M+H]+: 396.18

### Example 116

### 2-Chloro-N-(1-hydroxy-cyclohexylmethyl)-5-(2-methylamino-pyrimidin-4-yloxy)-benzamide

This compound was prepared using a method analogous to that of Example 14, intermediate 115.2 replacing intermediate 11.3 except that a 2M solution of methylamine in THF (2 eq) was used instead of a 41% solution in H₂O and that the reaction was stirred for 18 h at RT.

LC-MS (B): t_{R}=0.53 min; [M+H]+: 391.24

### Example 117

### 2-Chloro-5-(2-dimethylamino-pyrimidin-4-yloxy)-N-(1-hydroxy-cyclohexylmethyl)-benzamide

This compound was prepared using a method analogous to that of Example 15, intermediate 115.2 replacing intermediate 11.3 except that a 2M solution of dimethylamine in THF (2 eq) was used instead of a 40% solution in H₂O and that the reaction was stirred for 2 h at RT.

LC-MS (B): t_{R}=0.55 min; [M+H]+: 405.29

### Example 118

### 2-Chloro-N-(4,4-difluoro-1-hydroxy-cyclohexylmethyl)-5-(2-oxo-1,2-dihydro-pyrimidin-4-yloxy)-benzamide

### 118.1 4-Chloro-2-(2-trimethylsilanyl-ethoxy)-pyrimidine

To a solution of 2-(trimethylsilyl)-ethanol (0.962 mL) in THF (10 mL) was added dropwise a 2.5 M solution of n-butyllithium in hexanes at −70° C. The reaction mixture was allowed to warm up to −30° C. and cannulated in a solution of 2,4-dichloropyrimidine (1 g) in THF (10 mL) at −70° C. The reaction mixture was allowed to warm up to RT and stirred for 1 h at RT. It was quenched with ice H₂O and diluted with Et₂O. The organic phase was washed with a sat. solution of NaHCO₃, dried over MgSO₄ and concentrated in vacuo. The crude was purified by CC (Hept/EtOAc 1/0 to 9/1) to give 1.13 g of the titled compound as a light yellow oil.

¹H NMR ((CD₃)₂SO) δ: 8.58 (d, J=5.2 Hz, 1H), 7.29 (d, J=5.2 Hz, 1H), 4.43 (m, 2H), 1.12 (m, 2H), 0.06 (s, 9H)

### 118.2 2-Chloro-5-[2-(2-trimethylsilanyl-ethoxy)-pyrimidin-4-yloxy]-benzoic acid

This compound was prepared using a method analogous to that of Example 102 (intermediate 102.1), intermediate 118.1 replacing 2,6-difluoropyridine except that the reaction mixture was stirred for 1 h at 80° C.

LC-MS (B): t_{R}=0.87 min; [M-2Me+H]+: 339.03

### 118.3 2-Chloro-N-(4,4-difluoro-1-hydroxy-cyclohexylmethyl)-5-[2-(2-trimethylsilanyl-ethoxy)-pyrimidin-4-yloxy]-benzamide

This compound was prepared using a method analogous to that of Example 1 (intermediate 1.3), intermediate 118.2 replacing intermediate 1.2 and intermediate 57.2 replacing 1-aminomethyl-cyclohexanol hydrochloride.

LC-MS (B): t_{R}=0.91 min; [M-2Me+H]+: 486.14

### 118.4 2-Chloro-N-(4,4-difluoro-1-hydroxy-cyclohexylmethyl)-5-(2-oxo-1,2-dihydro-pyrimidin-4-yloxy)-benzamide

To a solution of intermediate 118.3 (110 mg) in DCM (4.8 mL) was added TFA (1.9 mL) and the reaction mixture was stirred for 30 min at RT. It was neutralized with a sat. solution of NaHCO₃ and concentrated in vacuo. The crude was purified by preparative LC-MS using method I to give 18 mg of the titled compound as a white powder.

LC-MS (B): t_{R}=0.54 min; [M+H]+: 413.98

### Example 119

### 2-Chloro-N-(1-hydroxy-cyclohexylmethyl)-5-(2-oxo-2,3-dihydro-pyrimidin-4-yloxy)-benzamide

This compound was prepared using a method analogous to that of Example 1 (intermediate 1.3), intermediate 118.2 replacing intermediate 1.2. The product was directly engaged in the next deprotection step using a method analogous to that of Example 118 (intermediate 118.4).

LC-MS (B): t_{R}=0.51 min; [M+H]+: 378.15

### Example 120

### 2-Chloro-5-(2-chloro-pyrimidin-4-yloxy)-N-(1-hydroxy-cyclohexylmethyl)-benzamide

### 120.1 2-Chloro-5-(2-methoxy-pyrimidin-4-yloxy)-benzoic acid

This compound was prepared using a method analogous to that of Example 102 (intermediate 102.1), 4-chloro-2-methoxypyrimidine replacing 2,6-difluoropyridine except that the reaction mixture was stirred for 1 h at 80° C. then for 18 h at RT and that the product precipitated out off the aqueous phase after acidification and was isolated by filtration.

LC-MS (B): t_{R}=0.60 min; [M+H]+: 281.07

### 120.2 2-Chloro-N-(1-hydroxy-cyclohexylmethyl)-5-(2-methoxy-pyrimidin-4-yloxy)-benzamide

This compound was prepared using a method analogous to that of Example 1 (intermediate 1.3), intermediate 120.1 replacing intermediate 1.2

LC-MS (B): t_{R}=0.66 min; [M+H]+: 392.06

### Example 121

### 2-Chloro-N-(1-hydroxy-cyclohexylmethyl)-5-(pyrazin-2-yloxy)-benzamide

### 121.1 2-Chloro-5-(pyrazin-2-yloxy)-benzoic acid

This compound was prepared using a method analogous to that of Example 102 (intermediate 102.1), 2-chloropyrazine replacing 2,6-difluoropyridine.

LC-MS (B): t_{R}=0.56 min; [M+CH₃CN+H]+: 292.26

### 121.2 2-Chloro-N-(1-hydroxy-cyclohexylmethyl)-5-(pyrazin-2-yloxy)-benzamide

This compound was prepared using a method analogous to that of Example 1 (intermediate 1.3), intermediate 121.1 replacing intermediate 1.2

LC-MS (B): t_{R}=0.63 min; [M+H]+: 362.10

### Example 122

### 2-Chloro-5-(6-chloro-pyrazin-2-yloxy)-N-(1-hydroxy-cyclohexyl methyl)-benzamide

### 122.1 2-Chloro-5-(6-chloro-pyrazin-2-yloxy)-benzoic acid

This compound was prepared using a method analogous to that of Example 102 (intermediate 102.1), 2,6-dichloropyrazine replacing 2,6-difluoropyridine.

LC-MS (B): t_{R}=0.68 min; [M+CH₃CN+H]+: 325.97

### 122.2 2-Chloro-5-(6-chloro-pyrazin-2-yloxy)-N-(1-hydroxy-cyclohexylmethyl)-benzamide

This compound was prepared using a method analogous to that of Example 1 (intermediate 1.3), intermediate 122.1 replacing intermediate 1.2

LC-MS (B): t_{R}=0.74 min; [M+H]+: 396.01

### Example 123

### 2-Chloro-N-(1-hydroxy-cyclohexylmethyl)-5-(6-methoxy-pyrazin-2-yloxy)-benzamide

This compound was prepared using a method analogous to that of Example 102 (intermediate 102.1), 2-chloro-6-methoxypyrazine replacing 2,6-difluoropyridine and intermediate 111.1 replacing 2-chloro-5-hydroxybenzoic acid.

LC-MS (B): t_{R}=0.71 min; [M+H]+: 392.08

### Example 124

### 2-Chloro-N-(1-hydroxy-cyclohexyl methyl)-5-(6-methylamino-pyrazin-2-yloxy)-benzamide

This compound was prepared using a method analogous to that of Example 14, intermediate 122.2 replacing intermediate 11.3 except that the reaction mixture was stirred for 18 h at 70° C.

LC-MS (B): t_{R}=0.64 min; [M+H]+: 391.07

### Example 125

### 2-Chloro-5-(6-dimethylamino-pyrazin-2-yloxy)-N-(1-hydroxy-cyclohexylmethyl)-benzamide

This compound was prepared using a method analogous to that of Example 15, intermediate 122.2 replacing intermediate 11.3 except that a 2M solution of dimethylamine in THF (10 eq) was used instead of a 40% solution in H₂O and that the reaction mixture was stirred for 18 h at 70° C.

LC-MS (B): t_{R}=0.70 min; [M+H]+: 405.12

### Example 126

### 2-Chloro-N-(1-hydroxy-cyclohexylmethyl)-5-(6-oxo-1,6-dihydro-pyrazin-2-yloxy)-benzamide

This compound was prepared using a method analogous to that of Example 16 (intermediate 16.1), intermediate 122.2 replacing intermediate 10.2 except that the reaction mixture was stirred for 15 min at 120° C. in a sealed vial and that the crude was purified by preparative LC-MS using method I.

LC-MS (B): t_{R}=0.61 min; [M+H]+: 378.08

### Example 127

### 2-Chloro-5-(6-chloro-pyridazin-3-yloxy)-N-(1-hydroxy-cyclohexylmethyl)-benzamide

This compound was prepared using a method analogous to that of Example 102 (intermediate 102.1), 3,6-dichloropyridazine replacing 2,6-difluoropyridine and intermediate 111.1 replacing 2-chloro-5-hydroxybenzoic acid.

LC-MS (B): t_{R}=0.67 min; [M+H]+: 396.03

### Example 128

### 2-Chloro-N-(1-hydroxy-cyclohexyl methyl)-5-(6-methoxy-pyridazin-3-yloxy)-benzamide

### 128.1 2-chloro-5-((6-methoxypyridazin-3-yl))oxy)benzoic acid

This compound was prepared using a method analogous to that of Example 102 (intermediate 102.1), 3-chloro-6-methoxypyridazine replacing 2,6-difluoropyridine except that the reaction mixture was stirred for 18 h at 120° C.

LC-MS (B): t_{R}=0.60 min; [M+H]+: 281.06

### 128.2 2-Chloro-N-(1-hydroxy-cyclohexylmethyl)-5-(6-methoxy-pyridazin-3-yloxy)-benzamide

This compound was prepared using a method analogous to that of Example 1 (intermediate 1.3), intermediate 128.1 replacing intermediate 1.2

LC-MS (B): t_{R}=0.66 min; [M+H]+: 392.11

### Example 129

### 2-Chloro-N-(1-hydroxy-cyclohexylmethyl)-5-(6-oxo-1,6-dihydro-pyridazin-3-yloxy)-benzamide

### 129.1 3-Chloro-6-(2-trimethylsilanyl-ethoxy)-pyridazine

To a solution of 2-(trimethylsilyl)-ethanol (0.505 mL) in THF (5 mL) was added portionwise a 60% dispersion of sodium hydride in mineral oil (148 mg) at 0° C. The suspension was stirred for 15 min at 0° C. then added to a solution of 3,6-dichloropyridazine (500 mg) in THF (5 mL) at 0° C. The reaction mixture was stirred for 30 min at 0° C. then for 18 h at RT. It was quenched with H₂O and a sat. solution of ammonium chloride and extracted with EtOAc. The organic phase was washed with brine, dried over MgSO₄ and concentrated. The crude was purified by CC (Hept/EtOAc 1/0 to 8/2) to give 497 mg of titled compound as a white solid.

¹H NMR ((CD₃)₂SO) δ: 7.77 (d, J=9.2 Hz, 1H), 7.29 (d, J=9.2 Hz, 1H), 4.52 (m, 2H), 1.15 (m, 2H), 0.07 (s, 9H).

### 129.2 2-Chloro-N-(1-hydroxy-cyclohexylmethyl)-5-[6-(2-trimethylsilanyl-ethoxy)-pyridazin-3-yloxy]-benzamide

This compound was prepared using a method analogous to that of Example 102 (intermediate 102.1), intermediate 129.1 replacing 2,6-difluoropyridine and intermediate 111.1 replacing 2-chloro-5-hydroxybenzoic acid except that the reaction mixture was stirred for 8 days at 80° C.

LC-MS (B): t_{R}=0.95 min; [M+H]+: 478.28

### 129.3 2-Chloro-N-(1-hydroxy-cyclohexylmethyl)-5-(6-oxo-1,6-dihydro-pyridazin-3-yloxy)-benzamide

To a solution of intermediate 129.2 (20 mg) in THF (0.2 mL) was added a 1M solution of tetrabutyl ammonium fluoride (0.126 mL) and the reaction mixture was stirred for 1 h at RT. It was concentrated in vacuo and purified by preparative LC-MS using method IV to give 2 mg of titled compound as a white powder.

LC-MS (B): t_{R}=0.55 min; [M+H]+: 378.07

### Example 130

### 2-Chloro-N-(1-hydroxy-cyclohexylmethyl)-5-(pyridazin-3-yloxy)-benzamide

### 130.1 3-Chloro-pyridazine

A suspension of 3(2H)-pyridazinone (1 g) in phosphorus oxychloride (0.970 mL) was heated to 80° C. for 18 h. The reaction mixture was evaporated off and the residue was treated with ice 2M solution of NaOH and extracted with EtOAc. The organic phase was washed with brine, dried over MgSO₄ and concentrated in vacuo to give 857 mg of the crude titled compound as a purple solid.

¹H NMR ((CD₃)₂SO) δ: 9.27 (d, J=4.7 Hz, 1H), 7.95 (d, J=8.7 Hz, 1H), 7.82 (dd, J₁=8.7 Hz, J₂=4.7 Hz, 1H)

### 130.2 2-Chloro-N-(1-hydroxy-cyclohexylmethyl)-5-(pyridazin-3-yloxy)-benzamide

This compound was prepared using a method analogous to that of Example 102 (intermediate 102.1), intermediate 130.1 replacing 2,6-difluoropyridine and intermediate 111.1 replacing 2-chloro-5-hydroxybenzoic acid.

LC-MS (B): t_{R}=0.58 min; [M+H]+: 362.14

### Example 131

### 2-Chloro-N-(1-hydroxy-cyclohexyl methyl)-5-(5-methoxy-pyridazin-3-yloxy)-benzamide

### 131.1 3-chloro-5-methoxypyridazine

To a solution of 3,5-dichloropyridazine (300 mg) in MeOH (2 mL) was added a 5.4 M solution of sodium methoxide in MeOH (0.410 mL) and the reaction mixture was stirred for 1 h at 90° C. It was quenched with H₂O and extracted with EtOAc. The organic phase was washed with a 5% solution of KHSO₄, a sat. solution of NaHCO₃ and brine, dried over MgSO₄ and concentrated in vacuo to give the crude titled compound as an orange solid.

¹H NMR ((CD₃)₂SO) δ: 9.01 (d, J=2.4 Hz, 1H), 7.55 (d, J=2.4 Hz, 1H), 3.96 (s, 3H)

### 131.2 2-Chloro-N-(1-hydroxy-cyclohexylmethyl)-5-(5-methoxy-pyridazin-3-yloxy)-benzamide

This compound was prepared using a method analogous to that of Example 102 (intermediate 102.1), intermediate 131.1 replacing 2,6-difluoropyridine and intermediate 111.1 replacing 2-chloro-5-hydroxybenzoic acid.

LC-MS (B): t_{R}=0.61 min; [M+H]+: 392.24

### Example 132

### 2-Chloro-N-(1-hydroxy-cyclohexylmethyl)-5-(pyridin-2-ylsulfanyl)-benzamide

### 132.1 2-Chloro-5-hydroxy-benzoic acid methyl ester

To a solution of 2-chloro-5-hydroxy-benzoic acid (3 g) in anh. MeOH (22.6 mL) was added a concentrated solution of sulfuric acid (0.870 mL) at RT and the reaction mixture was stirred for 18 h at 75° C. The solvent was evaporated off and the residue was diluted with H₂O and extracted with EtOAc. The organic phase was washed with a sat. solution of NaHCO₃, dried over MgSO₄ and concentrated in vacuo to give 3.15 g of the titled compound as a white solid.

¹H NMR (CDCl₃) δ: 7.36 (d, J=3.0 Hz, 1H), 7.29 (d, J=8.7 Hz, 1H), 6.96 (dd, J₁=8.7 Hz, J₂=3.0 Hz, 1H), 6.55 (s, 1H), 3.95 (s, 3H)

### 132.2 2-Chloro-5-dimethylthiocarbamoyloxy-benzoic acid methyl ester

To a solution of intermediate 132.1 (10 g) in 1-methyl-2-pyrrolidinone (8 mL) was added 1,4-diazabicyclo[2.2.2]octane (7.51 g) and the reaction mixture was heated to 50° C. A solution of dimethylthiocarbamoyl chloride (6.96 g) in 1-methyl-2-pyrrolidinone (2 mL) was added dropwise and the reaction mixture was stirred for 3 h at 50° C. It was quenched with H₂O (85 mL) over 10 min, heated to 50° C. and cooled to RT. It was extracted with EtOAc, the organic phase was dried over MgSO₄ and concentrated in vacuo. A light yellow solid precipitated out the oily residue and was filtered to give 10 g of the crude titled compound.

LC-MS (B): t_{R}=0.79 min; [M+H]+: 273.90

### 132.3 2-Chloro-5-dimethylcarbamoylsulfanyl-benzoic acid methyl ester

Intermediate 132.2 was heated to 220° C. and the melted solid was stirred for 24 h at 220° C. The cooled reaction mixture was purified by CC (Hept/EtOAc 1/0 to 7/3) to give 5.6 g of the titled compound as a yellow oil.

LC-MS (B): t_{R}=0.77 min; [M+H]+: 273.87

(ELN163-0487)

### 132.4 2-Chloro-5-mercapto-benzoic acid

To a solution of KOH (4.5 g) in THF (24 mL) was added a solution of intermediate 132.3 (5.5 g) in THF (2 mL) and the reaction mixture was stirred for 20 h at 70° C. The solvent was evaporated off and the residue was suspended in H₂O. The aqueous phase was acidified with a 2M solution of hydrochloric acid and extracted with DCM. The organic phase was dried over MgSO₄ and concentrated in vacuo to give 3.3 g of the crude titled compound as a light yellow oil.

¹H NMR (CDCl₃) δ: 7.95 (s, 1H), 7.39 (s, 2H), 3.59 (s, 1H)

### 132.5 2-chloro-5-(pyridin-2-ylsulfanyl)benzoic acid

This compound was prepared using a method analogous to that of Example 102 (intermediate 102.1), 2-chloropyridine replacing 2,6-difluoropyridine and intermediate 132.4 replacing 2-chloro-5-hydroxybenzoic acid except that the reaction mixture was stirred for 6 days at 80° C.

LC-MS (B): t_{R}=0.63 min; [M+H]+: 265.98

### 132.6 2-Chloro-N-(1-hydroxy-cyclohexylmethyl)-5-(pyridin-2-ylsulfanyl)-benzamide

This compound was prepared using a method analogous to that of Example 1 (intermediate 1.3), intermediate 132.5 replacing intermediate 1.2

LC-MS (B): t_{R}=0.69 min; [M+H]+: 377.07

### Example 133 rac-2-Chloro-N-(1-hydroxy-cyclohexylmethyl)-5-(pyridine-2-sulfinyl)-benzamide

To a solution of intermediate 132.6 (80 mg) in DCM (13 mL) was added dropwise a solution of 3-chloroperbenzoic acid (44 mg) in DCM (5 mL) at 0° C. for 20 min. The reaction mixture was stirred for 15 min at 0° C. and quenched with a sat. solution of KHSO₄. The organic phase was washed with a sat. solution of sodium carbonate and brine, dried over MgSO₄ and concentrated in vacuo. The crude was purified by CC (Hept/EtOAc 7/3 to 0/1) to give 59 mg of the titled compound as a white foam.

LC-MS (B): t_{R}=0.59 min; [M+H]+: 393.06

### Example 134

### 2-Chloro-N-(1-hydroxy-cyclohexylmethyl)-5-(pyridine-2-sulfonyl)-benzamide

To a solution of intermediate 132.6 (80 mg) in DCM (5 mL) was added 3-chloroperbenzoic acid (110 mg) and the reaction mixture was stirred for 1 h at RT. It was quenched with a sat. solution of NaHSO₄. The organic phase was washed with a sat. solution of sodium carbonate and brine, dried over MgSO₄ and concentrated in vacuo. The crude was purified by CC (Hept/EtOAc 0/1 to 2/8) to give 68 mg of the titled compound as a white foam.

LC-MS (B): t_{R}=0.64 min; [M+H]+: 409.06

### Example 135

### 2-Chloro-5-(2-chloro-pyrimidin-4-ylsulfanyl)-N-(1-hydroxy-cyclohexylmethyl)-benzamide

### 135.1 2-chloro-5-(2-chloropyrimidin-4-ylsulfanyl)benzoic acid

This compound was prepared using a method analogous to that of Example 102 (intermediate 102.1), 2,4-dichloropyrimidine replacing 2,6-difluoropyridine and intermediate 132.4 replacing 2-chloro-5-hydroxybenzoic acid except that the reaction mixture was stirred for 1 h at RT.

LC-MS (B): t_{R}=0.69 min; [M+H]+: 301.00

### 135.2 2-Chloro-5-(2-chloro-pyrimidin-4-ylsulfanyl)-N-(1-hydroxy-cyclohexylmethyl)-benzamide

This compound was prepared using a method analogous to that of Example 10 (intermediate 10.3), intermediate 135.1 replacing intermediate 10.2

LC-MS (B): t_{R}=0.74 min; [M+H]+: 411.91

### Example 136

### rac-2-Chloro-5-(2-chloro-pyrimidine-4-sulfinyl)-N-(1-hydroxy-cyclohexylmethyl)-benzamide

This compound was prepared using a method analogous to that of Example 133, intermediate 135.2 replacing intermediate 132.6

LC-MS (B): t_{R}=0.64 min; [M+H]+: 428.22

### Example 137

### 2-Chloro-N-(1-hydroxy-cyclohexylmethyl)-5-(2-oxo-1,2-dihydro-pyrimidin-4-ylsulfanyl)-benzamide

This compound was prepared using a method analogous to that of Example 16 (intermediate 16.1), intermediate 135.2 replacing intermediate 10.2 except that the reaction mixture was stirred for 10 min at 90° C. and that the crude was purified by preparative LC-MS using method III.

LC-MS (B): t_{R}=0.55 min; [M+H]+: 394.01

### Example 138

### 2-Chloro-N-(4,4-difluoro-1-hydroxy-cyclohexylmethyl)-5-(2-oxo-1,2-dihydro-pyrimidin-4-ylsulfanyl)-benzamide

### 138.1 2-Chloro-5-[2-(2-trimethylsilanyl-ethoxy)-pyrimidin-4-ylsulfanyl]-benzoic acid

This compound was prepared using a method analogous to that of Example 102 (intermediate 102.1), intermediate 118.1 replacing 2,6-difluoropyridine and intermediate 132.4 replacing 2-chloro-5-hydroxybenzoic acid except that the reaction mixture was stirred for 30 min at RT.

LC-MS (B): t_{R}=0.93 min; [M-2Me+H]+: 354.96

### 138.2 2-Chloro-N-(4,4-difluoro-1-hydroxy-cyclohexylmethyl)-5-[2-(2-trimethylsilanyl-ethoxy)-pyrimidin-4-ylsulfanyl]-benzamide

This compound was prepared using a method analogous to that of Example 1 (intermediate 1.3), intermediate 138.1 replacing intermediate 1.2 and intermediate 57.2 replacing 1-aminomethyl-cyclohexanol hydrochloride.

LC-MS (B): t_{R}=0.95 min; [M-2Me+H]+: 502.12

### 138.3 2-Chloro-N-(4,4-difluoro-1-hydroxy-cyclohexylmethyl)-5-(2-oxo-1,2-dihydro-pyrimidin-4-ylsulfanyn-benzamide

This compound was prepared using a method analogous to that of Example 118 (intermediate 118.4), intermediate 138.2 replacing intermediate 118.3.

LC-MS (B): t_{R}=0.55 min; [M+H]+: 429.94

### Example 139

### 2-Chloro-N-(1-hydroxy-cyclohexyl methyl)-5-(2-methoxy-pyrimidin-4-ylsulfanyl)-benzamide

### 139.1

### 2-Chloro-5-(2-methoxy-pyrimidin-4-ylsulfanyl)-benzoic acid

This compound was prepared using a method analogous to that of Example 102 (intermediate 102.1), 4-chloro-2-methoxypyrimidine replacing 2,6-difluoropyridine and intermediate 132.4 replacing 2-chloro-5-hydroxybenzoic acid except that the reaction mixture was stirred for 30 min at 80° C.

LC-MS (B): t_{R}=0.65 min; [M+H]+: 297.04

### 139.2 2-Chloro-N-(1-hydroxy-cyclohexylmethyl)-5-(2-methoxy-pyrimidin-4-ylsulfanyl)-benzamide

This compound was prepared using a method analogous to that of Example 1 (intermediate 1.3), intermediate 139.1 replacing intermediate 1.2

LC-MS (B): t_{R}=0.71 min; [M+H]+: 407.98

### Example 140

### rac-2-Chloro-N-(1-hydroxy-cyclohexylmethyl)-5-(2-methoxy-pyrimidine-4-sulfinyl)-benzamide

This compound was prepared using a method analogous to that of Example 133, intermediate 139.2 replacing intermediate 132.6 except that the reaction mixture was stirred for 3 h at 0° C. and an additional amount of 3-chloroperbenzoic acid (0.3 eq) was necessary for the completion of the reaction.

LC-MS (B): t_{R}=0.61 min; [M+H]+: 424.07

### Example 141

### 2-Chloro-N-(1-hydroxy-cyclohexyl methyl)-5-(2-methoxy-pyrimidine-4-sulfonyl)-benzamide

This compound was prepared using a method analogous to that of Example 134, intermediate 139.2 replacing intermediate 132.6 except that the reaction mixture was stirred for 18 h at RT and an additional amount of 3-chloroperbenzoic acid (2 eq) was necessary for the completion of the reaction.

LC-MS (B): t_{R}=0.67 min; [M+H]+: 440.07

### Example 142

### 2-Chloro-N-(1-hydroxy-cyclohexyl methyl)-5-(2-oxo-2H-pyridin-1-ylmethyl)-benzamide

### 142.1 5-Bromomethyl-2-chloro-benzoic acid

A suspension of 2-chloro-5-methylbenzoic acid (10 g) in chlorobenzene (200 mL) was heated to 50° C. and N-bromosuccinimide (10.95 g) was added. The reaction mixture was flushed with argon and 2,2′-azobis(2-methylpropionitrile) (98 mg) was added. The reaction mixture was refluxed for 4 h and 2,2′-azobis(2-methylpropionitrile) (98 mg) was added. The reaction mixture was refluxed for 1 h and stirred for 18 h at RT. The solvent was evaporated off and the residue taken up in Et₂O and filtered. The filtrate was washed with a 2M solution of hydrochloric acid and brine, dried over MgSO₄ and concentrated in vacuo. The crude was recrystallised from Et₂O/Hept to give 8 g of the titled compound as a beige solid.

¹H NMR ((CD₃)₂SO) δ: 13.51 (bs, 1H), 7.88 (d, J=2.2 Hz, 1H), 7.61 (dd, J₁=8.3 Hz, J₂=2.2 Hz, 1H), 7.55 (d, J=8.3 Hz, 1H), 4.76 (s, 2H)

### 142.2 5-(Benzotriazol-1-yloxymethyl)-2-chloro-N-(1-hydroxy-cyclohexylmethyl)-benzamide

This compound was prepared using a method analogous to that of Example 1 (intermediate 1.3), intermediate 142.1 replacing intermediate 1.2

LC-MS (B): t_{R}=0.72 min; [M+H]+: 415.28

### 142.3 2-Chloro-N-(1-hydroxy-cyclohexylmethyl)-5-(2-oxo-2H-pyridin-1-ylmethyl)-benzamide

To a solution of 2-hydroxypyridine (28 mg) in anh. 1,2-dimethoxyethane (1.6 mL) was added potassium carbonate (85 mg) and the suspension was refluxed for 1 h. Intermediate 142.2 (100 mg) was added in one portion and the reaction mixture was stirred for 20 h at 90° C. It was quenched with H₂O and extracted with EtOAc. The organic phase was washed with a sat. solution of NaHCO₃, a 5% solution of KHSO₄ and brine, dried over MgSO₄ and concentrated in vacuo. The crude was purified by CC (EtOAc/MeOH 1/0 to 8/2) to give 42 mg of titled compound as a white solid.

LC-MS (B): t_{R}=0.57 min; [M+H]+: 375.11

### Example 143

### 2-Chloro-N-(1-hydroxy-cyclohexylmethyl)-5-(2-oxo-2H-pyrimidin-1-ylmethyl)-benzamide

To a solution of 2-hydroxypyrimidine hydrochloride (58 mg) in anh. DMF (2 mL) was added potassium carbonate (127 mg) and sodium iodide (60 mg) and the suspension was refluxed for 1 h. Intermediate 142.2 (150 mg) was added in one portion and the reaction mixture was stirred for 20 h at 90° C. It was quenched with H₂O and extracted with DCM. The organic phase was washed with a sat. solution of NaHCO₃ dried over MgSO₄ and concentrated in vacuo. The crude was purified by CC (EtOAc/MeOH 1/0 to 8/2) to give 82 mg of titled compound as a white solid.

LC-MS (B): t_{R}=0.50 min; [M+H]+: 376.07

### Example 144

### 2-Chloro-5-(2,4-dioxo-3,4-dihydro-2H-pyrimidin-1-ylmethyl)-N-(1-hydroxy-cyclohexylmethyl)-benzamide

This compound was prepared using a method analogous to that of Example 143, uracil replacing 2-hydroxypyrimidine hydrochloride except that the reaction mixture was stirred for 4 days at 90° C. and an additional purification by preparative LC-MS using method IV was necessary.

LC-MS (B): t_{R}=0.57 min; [M+H]+: 392.26

### Example 145

### 2-Chloro-N-(1-hydroxy-cyclohexylmethyl)-5-pyridin-2-ylmethyl-benzamide

### 145.1 5-Bromomethyl-2-chloro-benzoic acid methyl ester

This compound was prepared using a method analogous to that of Example 132.1, intermediate 142.1 replacing 2-chloro-5-hydroxybenzoic acid except that the crude was purified by CC (Hept/EtOAc 1/0 to 85/15).

¹H NMR ((CD₃)₂SO) δ: 7.91 (d, J=2.2 Hz, 1H), 7.66 (dd, J₁=8.3 Hz, J₂=2.2 Hz, 1H), 7.59 (d, J=8.3 Hz, 1H), 4.77 (s, 2H), 3.88 (s, 3H)

### 145.2 2-chloro-5-(pyridin-2-ylmethyl)benzoic acid methyl ester

Preactivated zinc dust (500 mg) was suspended in THF (1 mL) and 3 drops of trimethylsilylchloride were added at RT followed by a solution of intermediate 145.1 (1 g) in THF (2 mL). The mixture was stirred for 10 min and added to a solution of 2-bromopyridine (0.453 mL) and tetrakis(triphenylphosphine)palladium(0) (27 mg) in THF (4 ml). The reaction mixture was stirred for 1 h at RT and the solvent was evaporated off. The residue was triturated in acetone and filtered. The filtrate was concentrated in vacuo and the crude was purified by CC (Hept/EtOAc 75/25 to 0/1) to give 197 mg of the titled compound as a yellowish oil.

LC-MS (B): t_{R}=0.49 min; [M+H]+: 262.05

### 145.3 2-chloro-5-(pyridin-2-ylmethyl)benzoic acid

This compound was prepared using a method analogous to that of Example 1 (intermediate 1.2), intermediate 145.2 replacing intermediate 1.1.

LC-MS (B): t_{R}=0.37 min; [M+H]+: 247.96

### 145.4 2-Chloro-N-(1-hydroxy-cyclohexylmethyl)-5-pyridin-2-ylmethyl-benzamide

This compound was prepared using a method analogous to that of Example 10 (intermediate 10.3), intermediate 145.3 replacing intermediate 10.2

LC-MS (B): t_{R}=0.49 min; [M+H]+: 359.12

### Example 146

### 2-Chloro-N-(1-hydroxy-cyclohexylmethyl)-5-(2-methylsulfanyl-pyrimidin-4-ylmethyl)-benzamide

### 146.1 2-chloro-5((2-methylsulfanylpyrimidin-4-yl)methyl)benzoic acid methyl ester

Dry lithium chloride (256 mg) and zinc dust (395 mg) were suspended in THF (0.8 mL) and 1,2-dibromoethane (0.013 mL) was added at RT. The reaction mixture was heated until ebullition and cooled to RT. Trimethylsilylchloride (0.004 mL) was added and the reaction mixture was heated until ebullition and cooled to 0° C. A solution of intermediate 145.1 (1.12 g) in THF (0.8 mL) was added and the reaction mixture was stirred for 30 min at RT. It was added dropwise to a solution of 4-iodo-2-methylsulfanylpyrimidine (747 mg), bis(dibenzylideneacetone)palladium (0) (43 mg) and tri-2-furylphosphine (34 mg) in THF (3 mL). The reaction mixture was stirred for 30 min at RT, quenched with a sat. solution of sodium carbonate and extracted with EtOAc. The organic phase was dried over MgSO₄ and concentrated in vacuo. The crude was purified by CC (Hept/EtOAc 9/1 to 6/4) to give 409 mg of the titled compound as a yellow oil.

LC-MS (B): t_{R}=0.83 min; [M+H]+: 309.10

### 146.2 2-chloro-5-((2-methylsulfanylpyrimidin-4-yl)methyl)benzoic acid

This compound was prepared using a method analogous to that of Example 1 (intermediate 1.2), intermediate 146.1 replacing intermediate 1.1 except that a 2M aqueous solution of NaOH was used instead of lithium hydroxide in H₂O.

LC-MS (B): t_{R}=0.68 min; [M+H]+: 295.09

### 146.3 2-Chloro-N-(1-hydroxy-cyclohexylmethyl)-5-(2-methylsulfanyl-pyrimidin-4-ylmethyl)-benzamide

This compound was prepared using a method analogous to that of Example 1 (intermediate 1.3), intermediate 146.2 replacing intermediate 1.2

LC-MS (B): t_{R}=0.73 min; [M+H]+: 406.30

### Example 147

### 2-Chloro-N-(1-hydroxy-cyclohexylmethyl)-5-(2-oxo-1,2-dihydro-pyrimidin-4-ylmethyl)-benzamide

### 147.1 2-chloro-N-(1-hydroxy-cyclohexylmethyl)-5-(2-methylsulfonylpyrimidin-4-yl-methyl)benzamide

To a solution of 3-chloroperbenzoic acid (395 mg) in THF (4 mL) was added dropwise at 0° C. a solution of intermediate 146.3 (310 mg) in DCM (0.8 mL). The reaction mixture was allowed to warm up to RT and stirred for 1 h at RT. The solvent was evaporated off and the residue was taken up in EtOAc. The organic phase was washed with a 10% solution of sodium carbonate, dried over MgSO₄ and concentrated in vacuo to give 475 mg of crude titled compound as a yellow waxy solid.

LC-MS (B): t_{R}=0.61 min; [M+H]+: 438.29

### 147.2 2-Chloro-N-(1-hydroxy-cyclohexylmethyl)-5-(2-oxo-1,2-dihydro-pyrimidin-4-ylmethyl)-benzamide

This compound was prepared using a method analogous to that of Example 16 (intermediate 16.1), intermediate 147.1 replacing intermediate 10.2 except that the reaction mixture was stirred for 1 h at RT and that the crude was purified first by CC (EtOAc/MeOH 1/0 to 8/2) and by preparative LC-MS using method V.

LC-MS (B): t_{R}=0.50 min; [M+H]+: 376.12

### Example 148

### 2-Chloro-N-(1-hydroxy-cyclohexyl methyl)-5-(2-methoxy-pyrimidin-4-ylmethyl)-benzamide

This compound was prepared using a method analogous to that of Example 12, intermediate 147.1 replacing intermediate 10.3 except that the reaction mixture was stirred for 6 h at RT and that the crude was purified by preparative LC-MS using method VI.

LC-MS (B): t_{R}=0.65 min; [M+H]+: 390.10

### Example 149

### 2-Chloro-N-(4,4-difluoro-1-hydroxy-cyclohexylmethyl)-5-(2-oxo-1,2-dihydro-pyrimidin-4-ylmethyl)-benzamide

### 149.1 2-Chloro-5-[2-(2-trimethylsilanyl-ethoxy)-pyrimidin-4-ylmethyl]-benzoic acid methyl ester

This compound was prepared using a method analogous to that of Example 146 (intermediate 146.1), intermediate 118.1 replacing 4-iodo-2-methylsulfanylpyrimidine except that the reaction mixture was stirred for 20 h at RT.

LC-MS (B): t_{R}=1.02 min; [M-2Me+H]+: 351.06

### 149.2 2-Chloro-5-[2-(2-trimethylsilanyl-ethoxy)-pyrimidin-4-ylmethyl]-benzoic acid

This compound was prepared using a method analogous to that of Example 1 (intermediate 1.2), intermediate 149.1 replacing intermediate 1.1.

LC-MS (B): t_{R}=0.89 min; [M-2Me+H]+: 337.07

### 149.3 2-Chloro-N-(4,4-difluoro-1-hydroxy-cyclohexylmethyl)-5-[2-(2-trimethylsilanyl-ethoxy)-pyrimidin-4-ylmethyl]-benzamide

This compound was prepared using a method analogous to that of Example 1 (intermediate 1.3), intermediate 149.2 replacing intermediate 1.2 and intermediate 57.2 replacing 1-aminomethyl-cyclohexanol hydrochloride.

LC-MS (B): t_{R}=0.92 min; [M-2Me+H]+: 484.14

### 149.4 2-Chloro-N-(4,4-difluoro-1-hydroxy-cyclohexylmethyl)-5-(2-oxo-1,2-dihydro-pyrimidin-4-ylmethyl)-benzamide

This compound was prepared using a method analogous to that of Example 118 (intermediate 118.4), intermediate 149.3 replacing intermediate 118.3 except that the reaction mixture was neutralised with Et₃N instead of a sat. solution of NaHCO₃.

LC-MS (B): t_{R}=0.50 min; [M+H]+: 412.26

### Example 150

### 2-Chloro-N-(1-hydroxy-cyclohexylmethyl)-5-(2-oxo-1,2-dihydro-pyrimidine-4-carbonyl)-benzamide

### 150.1 2-Chloro-5-cyanomethyl-benzoic acid methyl ester

To a suspension of intermediate 145.1 (2.5 g) and potassium carbonate (1.49 g) in CH₃CN (17 mL) was added trimethylsilylcyanide (1.72 mL) and the reaction mixture was stirred for 7 h at 60° C. and for 18 h at RT. Additional amount of trimethylsilylcyanide (0.86 mL) was added and the reaction mixture was stirred for 7 h at 60° C. and for 18 h at RT. It was quenched with a 1M solution of NaOH and extracted with toluene. The organic phase was washed with a 1M solution of NaOH and brine, dried over MgSO₄ and concentrated in vacuo. The crude was purified by CC (Hept/EtOAc 1/0 to 8/2) to give 1.59 g of the titled compound as a white solid.

¹H NMR ((CD₃)₂SO) δ: 7.82 (d, J=2.1 Hz, 1H), 7.64 (d, J=8.3 Hz, 1H), 7.57 (dd, J₁=8.3 Hz, J₂=2.1 Hz, 1H), 4.14 (s, 2H), 3.89 (s, 3H)

### 150.2 2-chloro-5-(2-(2-(trimethylsilyl)ethoxy)pyrimidine-4-carbonyl)benzoic acid sodium salt

To a solution of intermediate 150.1 (621 mg) and intermediate 118.1 (751 mg) in DMF (7.4 mL) was added at 0° C. under argon a 60% dispersion of sodium hydride in mineral oil (178 mg). The reaction mixture was allowed to warm up to RT and stirred for 20 min at RT. Additional amount of a 60% dispersion of sodium hydride in mineral oil (59 mg) was added at 0° C. under argon and the reaction mixture was stirred for 15 min at RT. It was diluted with DMF (5 mL) and additional amount of a 60% dispersion of sodium hydride in mineral oil (178 mg) was added. Compressed air was bubbled into the reaction mixture for 30 min and the reaction mixture was stirred for 5 days under air atmosphere. Additional amount of a 60% dispersion of sodium hydride in mineral oil (60 mg) was added at 0° C. and the reaction mixture was stirred for 18 h at RT under air atmosphere. It was quenched with a 20% aqueous solution of acetic acid, diluted with H₂O and extracted with EtOAc. The organic phase was washed with brine, dried over MgSO₄ and concentrated in vacuo. The crude was diluted with H₂O and a 2M solution of NaOH was added at RT. An ochre solid precipitated out of the aqueous phase and was filtered to give 792 mg of titled compound.

LC-MS (B): t_{R}=0.89 min; [M-2Me+H]+: 350.90

### 150.3 2-Chloro-N-(1-hydroxy-cyclohexylmethyl)-5-[2-(2-trimethylsilanyl-ethoxy)-pyrimidine-4-carbonyl]-benzamide

This compound was prepared using a method analogous to that of Example 1 (intermediate 1.3), intermediate 150.2 replacing intermediate 1.2.

LC-MS (B): t_{R}=0.94 min; [M+H]+: 489.92

### 150.4 2-Chloro-N-(1-hydroxy-cyclohexylmethyl)-5-(2-oxo-1,2-dihydro-pyrimidine-4-carbonyl)-benzamide

This compound was prepared using a method analogous to that of Example 118 (intermediate 118.4), intermediate 150.3 replacing intermediate 118.3 except that the reaction mixture was neutralised with Et₃N at 0° C. instead of a sat. solution of NaHCO₃.

LC-MS (B): t_{R}=0.52 min; [M+H]+: 389.91

### Example 151

### 2-Chloro-5-[difluoro-(2-oxo-1,2-dihydro-pyrimidin-4-yl)-methyl]-N-(1-hydroxy-cyclohexylmethyl)-benzamide

### 151.1 2-chloro-5-(2-(2-(trimethylsilyl)ethoxy)pyrimidine-4-carbonyl)benzoic acid methyl ester

This compound was prepared using a method analogous to that of Example 1 (intermediate 1.3), intermediate 150.2 replacing intermediate 1.2 and MeOH replacing 1-aminomethyl-cyclohexanol hydrochloride and DCM except that a catalytic amount of 4-dimethylaminopyridine (0.2 eq) was used.

LC-MS (B): t_{R}=1.04 min; [M-2Me+H]+: 364.93

### 151.2 2-Chloro-5-{difluoro-[2-(2-trimethylsilanyl-ethoxy)-pyrimidin-4-yl]-methyl}-benzoic acid methyl ester

A solution of intermediate 151.1 (63 mg) in bis(2-methoxyethyl)aminosulfur trifluoride (0.174 mL) was stirred for 20 h at 90° C. in a sealed vial. The reaction mixture was diluted with DCM and washed with H₂O, a sat. solution of NaHCO₃ and brine. The organic phase was dried over MgSO₄ and concentrated in vacuo. The crude was purified by CC (Hept/EtOAc 1/0 to 8/2) to give 16 mg of titled compound as a colorless oil.

LC-MS (B): t_{R}=1.07 min; [M-2Me+H]+: 386.93

### 151.3 2-Chloro-5-{difluoro-[2-(2-trimethylsilanyl-ethoxy)-pyrimidin-4-yl]-methyl}-benzoic acid

This compound was prepared using a method analogous to that of Example 1 (intermediate 1.2), intermediate 151.2 replacing intermediate 1.1.

LC-MS (B): t_{R}=0.95 min; [M-2Me+H]+: 372.99

### 151.4 2-Chloro-5-{difluoro-[2-(2-trimethylsilanyl-ethoxy)-pyrimidin-4-yl]-methyl}-N-(1-hydroxy-cyclohexylmethyl)-benzamide

This compound was prepared using a method analogous to that of Example 1 (intermediate 1.3), intermediate 151.3 replacing intermediate 1.2.

LC-MS (B): t_{R}=0.99 min; [M-2Me+H]+: 484.14

### 151.5 2-Chloro-5-[difluoro-(2-oxo-1,2-dihydro-pyrimidin-4-yl)-methyl]-N-(1-hydroxy-cyclohexylmethyl)-benzamide

This compound was prepared using a method analogous to that of Example 118 (intermediate 118.4), intermediate 151.4 replacing intermediate 118.3 except that the reaction mixture was neutralised with Et₃N at 0° C. instead of a sat. solution of NaHCO₃.

LC-MS (B): t_{R}=0.57 min; [M+H]+: 411.93

### Example 152 rac-2-Chloro-N-(1-hydroxy-cyclohexyl methyl)-5-[1-hydroxy-1-(2-oxo-1,2-dihydro-pyrimidin-4-yl)-ethyl]-benzamide

### 152.1 rac-2-Chloro-5-{hydroxy-[2-(2-trimethylsilanyl-ethoxy)-pyrimidin-4-yl]-methyl}-benzoic acid methyl ester

To a solution of intermediate 151.1 (196 mg) in MeOH (5 mL) was added sodium borohydride (23 mg) and the reaction mixture was stirred for 30 min at RT. It was quenched with H₂O and extracted with EtOAc. The organic phase was washed with brine, dried over MgSO₄ and concentrated in vacuo to give 192 mg of the crude titled compound as a colorless oil.

LC-MS (B): t_{R}=0.93 min; [M-2Me+H]+: 366.79

### 152.2 rac-2-Chloro-5-{fluoro-[2-(2-trimethylsilanyl-ethoxy)-pyrimidin-4-yl]-methyl}-benzoic acid methyl ester

To a solution of intermediate 152.1 (184 mg) in DCM (1 mL) was added at 0° C. a solution of bis(2-methoxyethyl)aminosulfur trifluoride (0.152 mL) in DCM (0.5 mL). The reaction mixture was stirred for 2 h at RT, quenched with a 5% solution of NaHCO₃, diluted with H₂O and extracted with DCM. The organic phase was dried over MgSO₄ and concentrated in vacuo. The crude was purified by CC (Hept/EtOAc 1/0 to 9/1) to give 48 mg of the titled compound as a colorless oil.

LC-MS (B): t_{R}=1.04 min; [M-2Me+H]+: 368.92

### 152.3 rac-2-Chloro-5-{fluoro-[2-(2-trimethylsilanyl-ethoxy)-pyrimidin-4-yl]-methyl}-benzoic acid

This compound was prepared using a method analogous to that of Example 1 (intermediate 1.2), intermediate 152.2 replacing intermediate 1.1.

LC-MS (B): t_{R}=0.91 min; [M-2Me+H]+: 354.91

### 152.4 rac-2-Chloro-5-{fluoro-[2-(2-trimethylsilanyl-ethoxy)-pyrimidin-4-yl]-methyl}-N-(1-hydroxy-cyclohexylmethyl)-benzamide

This compound was prepared using a method analogous to that of Example 1 (intermediate 1.3), intermediate 152.3 replacing intermediate 1.2.

LC-MS (B): t_{R}=0.95 min; [M-2Me+H]+: 465.72

### 152.5 rac-2-Chloro-5-[fluoro-(2-oxo-1,2-dihydro-pyrimidin-4-yl)-methyl]-N-(1-hydroxy-cyclohexylmethyl)-benzamide

This compound was prepared using a method analogous to that of Example 118 (intermediate 118.4), intermediate 152.4 replacing intermediate 118.3 except that the reaction mixture was neutralised with Et₃N at 0° C. instead of a sat. solution of NaHCO₃.

LC-MS (B): t_{R}=0.53 min; [M+H]+: 393.76

### Example 153

### 5-[(2-Chloro-pyrimidin-4-yl)-methyl-amino]-N-(1-hydroxy-cyclohexylmethyl)-2-methyl-benzamide

### 153.1 5-Amino-2-methyl-benzoic acid methyl ester

To a solution of 2-methyl-5-nitrobenzoic acid methyl ester (1015 mg) in EtOH (33 mL) was added 10% palladium on charcoal (175 mg) and the reaction mixture was stirred under an atmospheric pressure of hydrogen for 3 h at RT. It was filtered over a pad of celite and the filtrate was concentrated in vacuo to give 860 mg of the titled compound as an orange oil.

¹H NMR (CDCl₃) δ: 7.27 (d, J=2.6 Hz, 1H), 7.05 (d, J=8.1 Hz, 1H), 6.77 (dd, J₁=8.1 Hz, J₂=2.6 Hz, 1H), 3.89 (s, 3H), 3.64 (s, 2H), 2.49 (s, 3H)

### 153.2 5-((2-chloropyrimidin-4-yl)amino)-2-methylbenzoic acid methyl ester

This compound was prepared using a method analogous to that of Example 1, intermediate 1.1, 2,4-dichloropyrimidine replacing iodobenzene and intermediate 153.1 replacing methyl-5-amino-2-chlorobenzoate.

LC-MS (B): t_{R}=0.72 min; [M+H]+: 278.16

### 153.3 5-[(2-Chloro-pyrimidin-4-yl)-methyl-amino]-2-methyl-benzoic acid methyl ester

This compound was prepared using a method analogous to that of Example 11 (intermediate 11.1), intermediate 153.2 replacing intermediate 10.1.

LC-MS (B): t_{R}=0.78 min; [M+H]+: 292.16.

### 153.4 5-[(2-Chloro-pyrimidin-4-yl)-methyl-amino]-2-methyl-benzoic acid

This compound was prepared using a method analogous to that of Example 1 (intermediate 1.2), intermediate 153.3 replacing intermediate 1.1 except that a 2M aqueous solution of NaOH was used instead of lithium hydroxide in H₂O.

LC-MS (B): t_{R}=0.64 min; [M+H]+: 278.12

### 153.5 5-[(2-Chloro-pyrimidin-4-yl)-methyl-amino]-N-(1-hydroxy-cyclohexylmethyl)-2-methyl-benzamide

This compound was prepared using a method analogous to that of Example 10 (intermediate 10.3), intermediate 153.4 replacing intermediate 10.2.

LC-MS (B): t_{R}=0.67 min; [M+H]+: 389.33.

### Example 154

### N-(1-Hydroxy-cyclohexyl methyl)-5-[(2-methoxy-pyrimidin-4-yl)-methyl-amino]-2-methyl-benzamide

This compound was prepared using a method analogous to that of Example 12, intermediate 153.5 replacing intermediate 10.3 except that the reaction mixture was stirred for 5 h at 40° C.

LC-MS (B): t_{R}=0.51 min; [M+H]+: 385.22.

### Example 155

### N-(1-Hydroxy-cyclohexylmethyl)-2-methyl-5-[methyl-(2-oxo-1,2-dihydro-pyrimidin-4-yl)-amino]-benzamide

This compound was prepared using a method analogous to that of Example 16 (intermediate 16.1), intermediate 153.5 replacing intermediate 10.2 except that it was purified by CC (EtOAc/MeOH 1/0 to 8/2).

LC-MS (B): t_{R}=0.45 min; [M+H]+: 371.37.

### Example 156

### rac-2-Chloro-N-(1-hydroxy-cyclohexyl methyl)-5-[1-hydroxy-1-(2-oxo-1,2-dihydro-pyrimidin-4-yl)-ethyl]-benzamide

### 156.1 rac-2-Chloro-5-{1-hydroxy-1-[2-(2-trimethylsilanyl-ethoxy)-pyrimidin-4-yl]-ethyl}-benzoic acid methyl ester

To a solution of intermediate 151.1 (100 mg) in anh. THF (5 mL) was added dropwise at −10° C. a 3M solution of MeMgBr in Et₂O (0.17 mL). The reaction mixture was allowed to warm to RT and stirred for 30 min. It was cooled to 0° C., quenched with a sat. solution of NH₄Cl and extracted with EtOAc. The organic phase was washed with brine, dried over MgSO₄ and concentrated in vacuo to give 106 mg of the titled compound as a light yellow oil.

LC-MS (B): t_{R}=0.97 min; [M-2Me+H]+: 380.85

### 156.2 rac-2-Chloro-5-{1-hydroxy-1-[2-(2-trimethylsilanyl-ethoxy)-pyrimidin-4-yl]-ethyl}-benzoic acid

This compound was prepared using a method analogous to that of Example 1 (intermediate 1.2), intermediate 156.1 replacing intermediate 1.1.

LC-MS (B): t_{R}=0.85 min; [M-2Me+H]+: 366.96

### 156.3 rac-2-Chloro-N-(1-hydroxy-cyclohexylmethyl)-5-{1-hydroxy-1-[2-(2-trimethylsilanyl-ethoxy)-pyrimidin-4-yl]-ethyl}-benzamide

This compound was prepared using a method analogous to that of Example 1 (intermediate 1.3), intermediate 156.2 replacing intermediate 1.2.

LC-MS (B): t_{R}=0.90 min; [M-2Me+H]+: 477.94

### 156.4 rac-2-Chloro-N-(1-hydroxy-cyclohexylmethyl)-5-[1-hydroxy-1-(2-oxo-1,2-dihydro-pyrimidin-4-yl)-ethyl]-benzamide

This compound was prepared using a method analogous to that of Example 118 (intermediate 118.4), intermediate 156.3 replacing intermediate 118.3 except that the neutralization was performed using Et₃N.

LC-MS (B): t_{R}=0.50 min; [M+H]+: 406.29

### Example 157

### 2-Chloro-N-(1-hydroxy-cyclohexylmethyl)-5-[1-hydroxy-1-(2-oxo-1,2-dihydro-pyrimidin-4-yl)-ethyl]-benzamide (enantiomer A) and

### Example 158

### 2-Chloro-N-(1-hydroxy-cyclohexylmethyl)-5-[(1-hydroxy-1-(2-oxo-1,2-dihydro-pyrimidin-4-yl)-ethyl]-benzamide (enantiomer B)

Intermediate 156.4 was separated into the respective enantiomers using preparative chiral HPLC (Daicel, ChiralPak AD-H, 5 μm, 30×250 mm; Hept/EtOH 70/30, flow 34 mL/min), detection: UV 210 nm).

Both enantiomers were characterized by analytical chiral HPLC (Daicel, ChiralPak AD-H, 5 μm, 4.6×250 mm, Hept/EtOH 70/30, flow 0.8 mL/min), detection: UV 210 to 280 nm:

Enantiomer A: t_{R}=6.76 min (example 157)

Enantiomer B: t_{R}=9.50 min (example 158)

### Example 159

### rac-2-Chloro-N-(1-hydroxy-cyclohexylmethyl)-5-[1-hydroxy-1-(2-methoxy-pyrimidin-4-yl)-ethyl]-benzamide

### 159.1 2-Chloro-5-formyl-benzoic acid methyl ester

A mixture of intermediate 145.1 (250 mg) and methylmorpholine-4-oxide (315 mg) was suspended in dioxane (3 mL) and heated to reflux for 2 h. After cooling to RT, the reaction mixture was diluted with EtOAc and washed with an aqueous NH₄Cl solution, water and brine. The organic phase was dried over MgSO₄ and concentrated in vacuo to give 178 mg of the titled compound as an orange solid.

¹H NMR ((CD₃)₂SO) δ: 10.06 (s, 1H), 8.34 (d, J=2.0 Hz, 1H), 8.08 (dd, J₁=8.3 Hz, J₂=2.0 Hz, 1H), 7.85 (d, J=8.3 Hz, 1H), 3.92 (s, 3H)

### 159.2 2-Chloro-5-(2-methoxypyrimidine-4-carbonyl)benzoic acid methyl ester

A 60% suspension of NaH in mineral oil (51 mg) was added to a solution of 4-chloro-2-methoxypyrimidine (123 mg), intermediate 159.1 (170 mg) and dimethylimidazolium iodide (96 mg) in dioxane (35 mL). The reaction mixture was heated to reflux for 4 h30 and ON at RT. It was diluted with water and extracted with EtOAc. The organic phase was dried over MgSO₄ and concentrated in vacuo. The crude was purified by CC (Hept/EtOAc 1/0 to 7/3) to give 49 mg of the titled compound as a light yellow solid.

LC-MS (B): t_{R}=0.77 min; [M+H]+: 307.19

### 159.3 2-Chloro-5-(1-hydroxy-1-(2-methoxypyrimidin-4-yl)ethyl)benzoic acid methyl ester

This compound was prepared using a method analogous to that of Example 156 (intermediate 156.1), intermediate 159.2 replacing intermediate 151.1.

LC-MS (B): t_{R}=0.69 min; [M+H]+: 323.10

### 159.4 2-Chloro-5-(1-hydroxy-1-(2-methoxypyrimidin-4-yl)ethyl)benzoic acid

This compound was prepared using a method analogous to that of Example 1 (intermediate 1.2), intermediate 159.3 replacing intermediate 1.1.

LC-MS (B): t_{R}=0.55 min; [M+H]+: 309.09

### 159.5 rac-2-Chloro-N-(1-hydroxy-cyclohexylmethyl)-5-[1-hydroxy-1-(2-methoxy-pyrimidin-4-yl)-ethyl]-benzamide

This compound was prepared using a method analogous to that of Example 1 (intermediate 1.3), intermediate 159.4 replacing intermediate 1.2.

LC-MS (B): t_{R}=0.62 min; [M+H]+: 420.10

II. Biological assays

### In Vitro Assay

The P2X₇ receptor antagonistic activity of the compounds of formula (I) is determined in accordance with the following experimental method.

### Experimental Method:

### Cell Line Generation and YO-PRO Assay

Cell line generation was performed in general according to established molecular cloning protocols. Specifically, RNA was extracted from human whole blood using the Qiagen RNeasy kit (Qiagen, CH) according to the manufacturer's instructions. Subsequently cDNA was made (Superscript II, Invitrogen AG, CH) and the human P2X7 gene (genbank ref. BC011913) was amplified with the following primers: ATCGCGGCCGCTCAGTAAGGACTCTTGAAGCCACT and CGCCGCTAGCACCACCATGCCGGCCTGCTGCAGCTGCA. The amplified sequence was subsequently ligated into a pcDNA3.1 (+) NotI, NheI digested plasmid. Human embryonic kidney (HEK) cells (ATCC CRL—1573, Manassas, Va., USA) were transfected with the pcDNA3.1 (+).hP2X7 plasmid using lipofectamine 2000 (Invitrogen AG, CH) according to the manufacturer's instructions. Following a 24 h exposure to DNA, cells were trypsinized and re-seeded at low density in the presence of 250 μg Geneticin. Geneticin resistant cells were then selected during two consecutive rounds of cloning by serial limiting dilution with visual inspection. Individual clones were screened for P2X7 expression by applying ATP and recording the resultant uptake of YO-PRO1. Specific cell clones were chosen based on RNA and protein expression. HEK cells stably expressing P2X7 were used to screen drugs using the YO-PRO1 assay. Cells were grown to confluency in adherent culture at 37° C. in a humidified 5% CO₂ incubator (split 1/5 every 3-4 days with DMEM, 10% FCS, 1% Penicillin/Streptomycin, 250 μg/ml Geneticin).

Adherent cells were detached by incubation with Trypsine (1 ml per 165 cm² dish) for 2 minutes, then washed off with 10 ml PBS (without Mg²⁺ and Ca²⁺), and resuspended in DMEM, 10% FCS, 1% Penicillin/Streptomycin, no Geneticin. 10′000 cells per well (48 hours before the assay) or 25′000 cells per well (Vi-cell XR (Beckman Coulter) (24 hours before the assay) in 50 μl full medium were seeded on 384-well black-wall, clear bottom plates, that were coated before with 10 μl per well Poly-L-Lysine, incubated for 30-60 minutes at 37° C. and washed once with PBS. Medium was removed from cells and 50 μl of assay buffer containing 0.5 μM YO-PRO-1 was added into the wells. Solutions of antagonist compounds were prepared by serial dilutions of a 10 mM DMSO solution of the antagonist into PBS using a BioMek (Beckman Coulter). Each concentration was performed in duplicate. For IC₅₀ measurements 10 concentration points were measured (10 μM being the highest concentration followed by 9 serial dilution steps 1/3). The cells were incubated with the antagonists of the present invention together with ATP at a final concentration of 250 μM for 90 minutes. During this time period, four time points were taken. Each time point comprised the average of several measurements made within a few seconds. Fluorescence was measured in the FLIPR tetra (Molecular Devices) using the filters appropriate for YO-PRO-1 fluorescence (excitation 485/20, emission 530/25). The FLIPR tetra was equipped with Molecular Devices Screen Works system control software to define and run experimental protocols. For antagonist activity measurements, the maximal intensity was expressed as a percentage of that induced by the EC₅₀ value for agonist activation (0.25 mM ATP for HEK-293 cells expressing human recombinant P2X7 receptor). For IC50 measurements the maximum intensity is plotted against the concentration of compound to determine IC50 values.

Antagonistic activities with respect to the P2X₇ receptor (IC₅₀ values) of exemplified compounds are displayed in Table 1.

TABLE 1

| | IC₅₀ |
|---|---|
| Compound | [nM] |
| Example 1 | 111 |
| Example 2 | 315 |
| Example 3 | 538 |
| Example 4 | 103 |
| Example 5 | 531 |
| Example 6 | 154 |
| Example 7 | 876 |
| Example 8 | 459 |
| Example 9 | 481 |
| Example 10 | 890 |
| Example 11 | 184 |
| Example 12 | 733 |
| Example 13 | 200 |
| Example 14 | 127 |
| Example 15 | 177 |
| Example 16 | 73 |
| Example 17 | 35 |
| Example 18 | 106 |
| Example 19 | 420 |
| Example 20 | 228 |
| Example 21 | 17 |
| Example 22 | 267 |
| Example 23 | 18 |
| Example 24 | 236 |
| Example 25 | 109 |
| Example 26 | 317 |
| Example 27 | 30 |
| Example 28 | 353 |
| Example 29 | 456 |
| Example 30 | 181 |
| Example 31 | 566 |
| Example 32 | 531 |
| Example 33 | 183 |
| Example 34 | 860 |
| Example 35 | 367 |
| Example 36 | 185 |
| Example 37 | 464 |
| Example 38 | 764 |
| Example 39 | 378 |
| Example 40 | 588 |
| Example 41 | 384 |
| Example 42 | 38 |
| Example 43 | 136 |
| Example 44 | 33 |
| Example 45 | 89 |
| Example 46 | 21 |
| Example 47 | 116 |
| Example 48 | 15 |
| Example 49 | 1117 |
| Example 50 | 258 |
| Example 51 | 460 |
| Example 52 | 170 |
| Example 53 | 576 |
| Example 54 | 231 |
| Example 55 | 482 |
| Example 56 | 74 |
| Example 57 | 214 |
| Example 58 | 51 |
| Example 59 | 1810 |
| Example 60 | 1027 |
| Example 61 | 681 |
| Example 62 | 416 |
| Example 63 | 307 |
| Example 64 | 902 |
| Example 65 | 205 |
| Example 66 | 158 |
| Example 67 | 337 |
| Example 68 | 288 |
| Example 69 | 326 |
| Example 70 | 373 |
| Example 71 | 1997 |
| Example 72 | 1460 |
| Example 73 | 322 |
| Example 74 | 280 |
| Example 75 | 1023 |
| Example 76 | 418 |
| Example 77 | 1463 |
| Example 78 | 439 |
| Example 79 | 352 |
| Example 80 | 1433 |
| Example 81 | 245 |
| Example 82 | 76 |
| Example 83 | 57 |
| Example 84 | 65 |
| Example 85 | 177 |
| Example 86 | 61 |
| Example 87 | 190 |
| Example 88 | 302 |
| Example 89 | 271 |
| Example 90 | 905 |
| Example 91 | 354 |
| Example 92 | 82 |
| Example 93 | 111 |
| Example 94 | 160 |
| Example 95 | 351 |
| Example 96 | 123 |
| Example 97 | 317 |
| Example 98 | 332 |
| Example 99 | 148 |
| Example 100 | 217 |
| Example 101 | 319 |
| Example 102 | 37 |
| Example 103 | 46 |
| Example 104 | 100 |
| Example 105 | 47 |
| Example 106 | 1715 |
| Example 107 | 70 |
| Example 108 | 64 |
| Example 109 | 53 |
| Example 110 | 122 |
| Example 111 | 21 |
| Example 112 | 43 |
| Example 113 | 96 |
| Example 114 | 100 |
| Example 115 | 191 |
| Example 116 | 172 |
| Example 117 | 427 |
| Example 118 | 14 |
| Example 119 | 796 |
| Example 120 | 111 |
| Example 121 | 93 |
| Example 122 | 101 |
| Example 123 | 123 |
| Example 124 | 163 |
| Example 125 | 504 |
| Example 126 | 461 |
| Example 127 | 58 |
| Example 128 | 30 |
| Example 129 | 80 |
| Example 130 | 67 |
| Example 131 | 206 |
| Example 132 | 46 |
| Example 133 | 265 |
| Example 134 | 105 |
| Example 135 | 266 |
| Example 136 | 640 |
| Example 137 | 8.1 |
| Example 138 | 10 |
| Example 139 | 74 |
| Example 140 | 914 |
| Example 141 | 3895 |
| Example 142 | 80 |
| Example 143 | 377 |
| Example 144 | 1390 |
| Example 145 | 21 |
| Example 146 | 400 |
| Example 147 | 123 |
| Example 148 | 343 |
| Example 149 | 33 |
| Example 150 | 77 |
| Example 151 | 31 |
| Example 152 | 25 |
| Example 153 | 702 |
| Example 154 | 629 |
| Example 155 | 199 |
| Example 156 | 14 |
| Example 157 | 9.0 |
| Example 158 | 66 |
| Example 159 | 346 |

1. A compound of the formula (I), 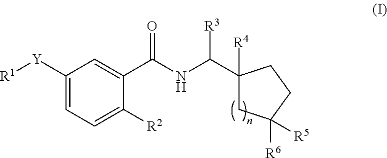
wherein
n represents 1, 2, 3 or 4 (and preferably 2, 3 or 4);
Y represents —C(R⁷R⁸)—, —N(R⁹)—, —O—, —S—, —S(O)—, or —S(O)₂—;
R¹ represents
a 5-membered heteroaryl group which is unsubstituted or mono- or di-substituted with (C₁-C₄)alkyl;
a 6-membered heteroaryl group which is unsubstituted or mono- or di-substituted, wherein the substituents are independently selected from the group consisting of halogen, hydroxy, (C₁-C₄)alkyl, (C₁-C₄)alkoxy, (C₁-C₄)alkylthio, (C₁-C₄)alkyl-sulfonyl, (C₁-C₄)alkyl-amino and di-[(C₁-C₄)alkyl]-amino;
a phenyl group which is unsubstituted or mono- or di-substituted with halogen; or
a heterocyclyl group which is unsubstituted or mono- or di-substituted with (C₁-C₄)alkyl or (C₁-C₂)alkoxy-(C₁-C₄)alkyl;
R² represents chloro or methyl (and preferably chloro);
R³ represents hydrogen and R⁴ represents hydroxy, hydroxy-(C₁-C₄)alkyl, —CONH₂ or (C₁-C₄)alkoxy (and preferably hydroxy, hydroxymethyl or methoxy); or
R³ represents (C₁-C₄)alkyl or hydroxy-(C₁-C₄)alkyl (and preferably methyl or hydroxymethyl) and R⁴ represents hydrogen;
R⁵ represents hydrogen or fluoro;
R⁶ represents hydrogen or fluoro;
R⁷ and R⁸ represent independently from each other hydrogen, fluoro, hydroxy or (C₁-C₄)alkyl, with the proviso that R⁸ is different from fluoro or hydroxy if R⁷ represents hydroxy;
or R⁷ and R⁸ together represent an oxo-group; and
R⁹ represents hydrogen, (C₁-C₄)alkyl, (C₁-C₂)alkoxy-(C₁-C₄)alkyl, (C₃-C₆)cycloalkyl-(C₁-C₄)alkyl, phenyl-(C₁-C₄)alkyl, or phenyloxy-(C₁-C₄)alkyl;
or a salt of such a compound.

2. A compound of formula (I) according to claim 1, wherein
n represents 1, 2, 3 or 4;
Y represents —C(R⁷R⁸)—, —N(R⁹)—, —O—, —S—, —S(O)—, or —S(O)₂—;
R¹ represents
a 5-membered heteroaryl group which is unsubstituted or mono- or di-substituted with (C₁-C₄)alkyl;
a 6-membered heteroaryl group which is unsubstituted or mono- or di-substituted, wherein the substituents are independently selected from the group consisting of halogen, hydroxy, (C₁-C₄)alkyl, (C₁-C₄)alkoxy, (C₁-C₄)alkylthio, (C₁-C₄)alkyl-sulfonyl, (C₁-C₄)alkyl-amino and di-[(C₁-C₄)alkyl]-amino;
a phenyl group which is unsubstituted or mono- or di-substituted with halogen; or
a heterocyclyl group which is unsubstituted or mono- or di-substituted with (C₁-C₄)alkyl or (C₁-C₂)alkoxy-(C₁-C₄)alkyl;
R² represents chloro or methyl;
R³ represents hydrogen and R⁴ represents hydroxy, hydroxy-(C₁-C₄)alkyl, —CONH₂ or (C₁-C₄)alkoxy;
R⁵ represents hydrogen or fluoro;
R⁶ represents hydrogen or fluoro;
R⁷ and R⁸ represent independently from each other hydrogen or fluoro; or R⁷ and R⁸ together represent an oxo-group; and
R⁹ represents hydrogen, (C₁-C₄)alkyl, (C₁-C₂)alkoxy-(C₁-C₄)alkyl, (C₃-C₆)cycloalkyl-(C₁-C₄)alkyl, phenyl-(C₁-C₄)alkyl, or phenyloxy-(C₁-C₄)alkyl;
or a salt of such a compound.

3. A compound of formula (I) according to claim 1, wherein
n represents 1, 2, 3 or 4;
Y represents —C(R⁷R⁸)—, —N(R⁹)—, —O—, or —S—;
R¹ represents a 6-membered heteroaryl group which is unsubstituted or mono- or di-substituted, wherein the substituents are independently selected from the group consisting of halogen, hydroxy, (C₁-C₄)alkyl, (C₁-C₄)alkoxy, (C₁-C₄)alkylthio, (C₁-C₄)alkyl-sulfonyl, (C₁-C₄)alkyl-amino and di-[(C₁-C₄)alkyl]-amino;
R² represents chloro;
R³ represents (C₁-C₄)alkyl or hydroxy-(C₁-C₄)alkyl and R⁴ represents hydrogen;
R⁵ represents hydrogen or fluoro;
R⁶ represents hydrogen or fluoro;
R⁷ and R⁸ represent independently from each other hydrogen or fluoro; or R⁷ and R⁸ together represent an oxo-group; and
R⁹ represents hydrogen, (C₁-C₄)alkyl, (C₁-C₂)alkoxy-(C₁-C₄)alkyl, (C₃-C₆)cycloalkyl-(C₁-C₄)alkyl, phenyl-(C₁-C₄)alkyl, or phenyloxy-(C₁-C₄)alkyl;
or a salt of such a compound.

4. A compound of formula (I) according to claim 1, wherein
n represents 2, 3 or 4;
or a salt of such a compound.

5. A compound of formula (I) according to claim 1, wherein
Y represents —C(R⁷R⁸)—, —O—, or —S—;
or a salt of such a compound.

6. A compound of formula (I) according to claim 1, wherein
Y represents —N(R⁹)—;
or a salt of such a compound.

7. A compound of formula (I) according to claim 1, wherein
R¹ represents a 6-membered heteroaryl group which is unsubstituted or mono- or di-substituted, wherein the substituents are independently selected from the group consisting of halogen, hydroxy, (C₁-C₄)alkyl, (C₁-C₄)alkoxy, (C₁-C₄)alkylthio, (C₁-C₄)alkyl-sulfonyl, (C₁-C₄)alkyl-amino and di-[(C₁-C₄)alkyl]-amino;
or a salt of such a compound.

8. A compound of formula (I) according to claim 1, wherein
R² represents chloro;
or a salt of such a compound.

9. A compound of formula (I) according to claim 1, wherein
R³ represents hydrogen and R⁴ represents hydroxy or hydroxy-(C₁-C₄)alkyl;
or a salt of such a compound.

10. A compound of formula (I) according to claim 1, wherein
R³ represents (C₁-C₄)alkyl or hydroxy-(C₁-C₄)alkyl and R⁴ represents hydrogen;
or a salt of such a compound.

11. A compound of formula (I) according to claim 1, selected from the group consisting of:
2-Chloro-N-(1-hydroxy-cyclohexylmethyl)-5-phenylamino-benzamide;
2-Chloro-N-((1-hydroxycyclohexyl)methyl)-5-(methyl(phenyl)amino)benzamide;
2-Chloro-N-(1-hydroxy-cyclohexylmethyl)-5-[(2-methoxy-ethyl)-phenyl-amino]-benzamide;
2-Chloro-5-(2-fluoro-phenylamino)-N-(1-hydroxy-cyclohexylmethyl)-benzamide;
2-Chloro-5-[(2-fluoro-phenyl)-methyl-amino]-N-(1-hydroxy-cyclohexylmethyl)-benzamide;
2-Chloro-5-(2,4-difluoro-phenylamino)-N-(1-hydroxy-cyclohexylmethyl)-benzamide;
2-Chloro-5-[(2,4-difluoro-phenyl)-methyl-amino]-N-(1-hydroxy-cyclohexylmethyl)-benzamide;
2-Chloro-N-(1-hydroxy-cyclohexylmethyl)-5-(pyrimidin-4-ylamino)-benzamide;
2-Chloro-N-(1-hydroxy-cyclohexylmethyl)-5-(methyl-pyrimidin-4-yl-amino)-benzamide;
2-Chloro-5-(2-chloro-pyrimidin-4-ylamino)-N-(1-hydroxy-cyclohexylmethyl)-benzamide;
2-Chloro-5-[(2-chloro-pyrimidin-4-yl)-methyl-amino]-N-(1-hydroxy-cyclohexylmethyl)-benzamide;
2-Chloro-N-(1-hydroxy-cyclohexylmethyl)-5-(2-methoxy-pyrimidin-4-ylamino)-benzamide;
2-Chloro-N-(1-hydroxy-cyclohexylmethyl)-5-[(2-methoxy-pyrimidin-4-yl)-methyl-amino]-benzamide;
2-Chloro-N-(1-hydroxy-cyclohexylmethyl)-5-[methyl-(2-methylamino-pyrimidin-4-yl)-amino]-benzamide;
2-Chloro-5-[(2-dimethylamino-pyrimidin-4-yl)-methyl-amino]-N-(1-hydroxy-cyclohexylmethyl)-benzamide;
2-Chloro-N-(1-hydroxy-cyclohexylmethyl)-5-(2-hydroxypyrimidin-4-ylamino)-benzamide;
2-Chloro-N-(1-hydroxy-cyclohexylmethyl)-5-[methyl-(2-oxo-1,2-dihydro-pyrimidin-4-yl)-amino]-benzamide;
2-Chloro-N-(1-hydroxy-cyclohexylmethyl)-5-[methyl-(3-methyl-2-oxo-2,3-dihydro-pyrimidin-4-yl)-amino]-benzamide;
2-Chloro-N-(1-hydroxy-cyclohexylmethyl)-5-[methyl-(1-methyl-2-oxo-1,2-dihydro-pyrimidin-4-yl)-amino]-benzamide;
2-Chloro-5-[(2-chloro-pyrimidin-4-yl)-ethyl-amino]-N-(1-hydroxy-cyclohexylmethyl)-benzamide;
2-Chloro-5-[ethyl-(2-oxo-1,2-dihydro-pyrimidin-4-yl)-amino]-N-(1-hydroxy-cyclohexylmethyl)-benzamide;
2-Chloro-5-[(2-chloro-pyrimidin-4-yl)-propyl-amino]-N-(1-hydroxy-cyclohexylmethyl)-benzamide;
2-Chloro-N-(1-hydroxy-cyclohexylmethyl)-5-[(2-oxo-1,2-dihydro-pyrimidin-4-yl)-propyl-amino]-benzamide;
2-Chloro-5-[(2-chloro-pyrimidin-4-yl)-isopropyl-amino]-N-(1-hydroxy-cyclohexylmethyl)-benzamide;
2-Chloro-N-(1-hydroxy-cyclohexylmethyl)-5-[isopropyl-(2-oxo-1,2-dihydro-pyrimidin-4-yl)-amino]-benzamide;
2-Chloro-5-[(2-chloro-pyrimidin-4-yl)-isobutyl-amino]-N-(1-hydroxy-cyclohexylmethyl)-benzamide;
2-Chloro-N-(1-hydroxy-cyclohexylmethyl)-5-[isobutyl-(2-oxo-1,2-dihydro-pyrimidin-4-yl)-amino]-benzamide;
2-Chloro-N-(1-hydroxy-cyclohexylmethyl)-5-[isobutyl-(2-methoxy-pyrimidin-4-yl)-amino]-benzamide;
5-[Benzyl-(2-chloro-pyrimidin-4-yl)-amino]-2-chloro-N-(1-hydroxy-cyclohexylmethyl)-benzamide;
5-[Benzyl-(2-oxo-1,2-dihydro-pyrimidin-4-yl)-amino]-2-chloro-N-(1-hydroxy-cyclohexylmethyl)-benzamide;
5-[Benzyl-(2-methoxy-pyrimidin-4-yl)-amino]-2-chloro-N-(1-hydroxy-cyclohexylmethyl)-benzamide;
2-Chloro-5-[(2-chloro-pyrimidin-4-yl)-(2-methoxy-ethyl)-amino]-N-(1-hydroxy-cyclohexylmethyl)-benzamide;
2-Chloro-N-(1-hydroxy-cyclohexylmethyl)-5-[(2-methoxy-ethyl)-(2-oxo-1,2-dihydro-pyrimidin-4-yl)-amino]-benzamide;
2-Chloro-N-(1-hydroxy-cyclohexylmethyl)-5-[(2-methoxy-ethyl)-(2-methoxy-pyrimidin-4-yl)-amino]-benzamide;
2-Chloro-5-[(2-chloro-pyrimidin-4-yl)-cyclopentylmethyl-amino]-N-(1-hydroxy-cyclohexylmethyl)-benzamide;
2-Chloro-5-[cyclopentylmethyl-(2-oxo-1,2-dihydro-pyrimidin-4-yl)-amino]-N-(1-hydroxy-cyclohexylmethyl)-benzamide;
2-Chloro-5-[cyclopentylmethyl-(2-methoxy-pyrimidin-4-yl)-amino]-N-(1-hydroxy-cyclohexylmethyl)-benzamide;
2-Chloro-5-[(2-chloro-pyrimidin-4-yl)-(2-phenoxy-ethyl)-amino]-N-(1-hydroxy-cyclohexylmethyl)-benzamide;
2-Chloro-N-(1-hydroxy-cyclohexylmethyl)-5-[(2-oxo-1,2-dihydro-pyrimidin-4-yl)-(2-phenoxy-ethyl)-amino]-benzamide;
2-Chloro-N-(1-hydroxy-cyclohexylmethyl)-5-[(2-methoxy-pyrimidin-4-yl)-(2-phenoxy-ethyl)-amino]-benzamide;
2-Chloro-5-[(2-chloro-pyrimidin-4-yl)-methyl-amino]-N—((R)-1-cyclohexyl-ethyl)-benzamide;
2-Chloro-N—((R)-1-cyclohexyl-ethyl)-5-[methyl-(2-oxo-1,2-dihydro-pyrimidin-4-yl)-amino]-benzamide;
2-Chloro-5-[(2-chloro-pyrimidin-4-yl)-methyl-amino]-N—((S)-1-cyclohexyl-2-hydroxy-ethyl)-benzamide;
2-Chloro-N—((S)-1-cyclohexyl-2-hydroxy-ethyl)-5-[methyl-(2-oxo-1,2-dihydro-pyrimidin-4-yl)-amino]-benzamide;
2-Chloro-5-[(2-chloro-pyrimidin-4-yl)-methyl-amino]-N-(1-hydroxy-cycloheptylmethyl)-benzamide;
2-Chloro-N-(1-hydroxy-cycloheptylmethyl)-5-[methyl-(2-oxo-1,2-dihydro-pyrimidin-4-yl)-amino]-benzamide;
2-Chloro-5-[(2-chloro-pyrimidin-4-yl)-methyl-amino]-N-(1-hydroxy-cyclooctylmethyl)-benzamide;
2-Chloro-N-(1-hydroxy-cyclooctylmethyl)-5-[methyl-(2-oxo-2,3-dihydro-pyrimidin-4-yl)-amino]-benzamide;
2-Chloro-5-[(2-chloro-pyrimidin-4-yl)-methyl-amino]-N-(1-hydroxy-cyclopentylmethyl)-benzamide;
2-Chloro-N-(1-hydroxy-cyclopentylmethyl)-5-[methyl-(2-oxo-2,3-dihydro-pyrimidin-4-yl)-amino]-benzamide;
2-Chloro-5-[(2-chloro-pyrimidin-4-yl)-methyl-amino]-N-(1-methoxy-cyclohexylmethyl)-benzamide;
2-Chloro-N-(1-methoxy-cyclohexylmethyl)-5-[methyl-(2-oxo-2,3-dihydro-pyrimidin-4-yl)-amino]-benzamide;
N-(1-Carbamoyl-cyclohexylmethyl)-2-chloro-5-[(2-chloro-pyrimidin-4-yl)-methyl-amino]-benzamide;
N-(1-Carbamoyl-cyclohexylmethyl)-2-chloro-5-[methyl-(2-oxo-2,3-dihydro-pyrimidin-4-yl)-amino]-benzamide;
2-Chloro-5-[(2-chloro-pyrimidin-4-yl)-methyl-amino]-N-(1-hydroxymethyl-cyclohexylmethyl)-benzamide;
2-Chloro-N-(1-hydroxymethyl-cyclohexylmethyl)-5-[methyl-(2-oxo-2,3-dihydro-pyrimidin-4-yl)-amino]-benzamide;
2-Chloro-5-[(2-chloro-pyrimidin-4-yl)-methyl-amino]-N-(4,4-difluoro-1-hydroxy-cyclohexylmethyl)-benzamide;
2-Chloro-N-(4,4-difluoro-1-hydroxy-cyclohexylmethyl)-5-[methyl-(2-oxo-2,3-dihydro-pyrimidin-4-yl)-amino]-benzamide;
2-Chloro-N—((R)-1-cyclohexyl-ethyl)-5-(2-methylsulfanyl-pyrimidin-4-ylamino)-benzamide;
2-Chloro-N—((S)-1-cyclohexyl-2-hydroxy-ethyl)-5-(2-methylsulfanyl-pyrimidin-4-ylamino)-benzamide;
2-Chloro-N-(1-hydroxy-cycloheptylmethyl)-5-(2-methylsulfanyl-pyrimidin-4-ylamino)-benzamide;
2-Chloro-5-[(2,6-dichloro-pyrimidin-4-yl)-methyl-amino]-N-(1-hydroxy-cyclohexylmethyl)-benzamide;
2-Chloro-5-[(6-chloro-2-methoxy-pyrimidin-4-yl)-methyl-amino]-N-(1-hydroxy-cyclohexylmethyl)-benzamide;
2-Chloro-5-[(6-chloro-2-oxo-1,2-dihydro-pyrimidin-4-yl)-methyl-amino]-N-(1-hydroxy-cyclohexylmethyl)-benzamide;
2-Chloro-N-(1-hydroxy-cyclohexylmethyl)-5-[methyl-(6-methylsulfanyl-pyrimidin-4-yl)-amino]-benzamide;
2-Chloro-N-(1-hydroxy-cyclohexylmethyl)-5-[(6-methanesulfonyl-pyrimidin-4-yl)-methyl-amino]-benzamide;
2-Chloro-N-(1-hydroxy-cyclohexylmethyl)-5-[(6-methoxy-pyrimidin-4-yl)-methyl-amino]-benzamide;
2-Chloro-N-(1-hydroxy-cyclohexylmethyl)-5-[methyl-(6-oxo-1,6-dihydro-pyrimidin-4-yl)-amino]-benzamide;
2-Chloro-N-(1-hydroxy-cyclohexylmethyl)-5-(pyrimidin-2-ylamino)-benzamide;
2-Chloro-N-(1-hydroxy-cyclohexylmethyl)-5-(methyl-pyrimidin-2-yl-amino)-benzamide;
2-Chloro-N-(1-hydroxy-cyclohexylmethyl)-5-[(2-methoxy-ethyl)-pyrimidin-2-yl-amino]-benzamide;
2-Chloro-N-(1-hydroxy-cyclohexylmethyl)-5-(4-methylsulfanyl-pyrimidin-2-ylamino)-benzamide;
2-Chloro-N-(1-hydroxy-cyclohexylmethyl)-5-[methyl-(4-methylsulfanyl-pyrimidin-2-yl)-amino]-benzamide;
2-Chloro-N-(1-hydroxy-cyclohexylmethyl)-5-[(4-methoxy-pyrimidin-2-yl)-methyl-amino]-benzamide;
2-Chloro-N-(1-hydroxy-cyclohexylmethyl)-5-[methyl-(6-oxo-1,6-dihydro-pyrimidin-2-yl)-amino]-benzamide;
2-Chloro-N-(1-hydroxy-cyclohexylmethyl)-5-(pyrimidin-5-ylamino)-benzamide;
2-Chloro-N-(1-hydroxy-cyclohexylmethyl)-5-[(2-methoxy-ethyl)-pyrimidin-5-yl-amino]-benzamide;
2-Chloro-N-(1-hydroxy-cyclohexylmethyl)-5-(pyrazin-2-ylamino)-benzamide;
2-Chloro-N-(1-hydroxy-cyclohexylmethyl)-5-(methyl-pyrazin-2-yl-amino)-benzamide;
2-Chloro-N-(1-hydroxy-cyclohexylmethyl)-5-[(2-methoxy-ethyl)-pyrazin-2-yl-amino]-benzamide;
2-Chloro-5-[(6-chloro-pyrazin-2-yl)-methyl-amino]-N-(1-hydroxy-cyclohexylmethyl)-benzamide;
2-Chloro-N-(1-hydroxy-cyclohexylmethyl)-5-[(6-methoxy-pyrazin-2-yl)-methyl-amino]-benzamide;
2-Chloro-N-(1-hydroxy-cyclohexylmethyl)-5-[methyl-(6-methylamino-pyrazin-2-yl)-amino]-benzamide;
2-Chloro-5-[(6-dimethylamino-pyrazin-2-yl)-methyl-amino]-N-(1-hydroxy-cyclohexylmethyl)-benzamide;
2-Chloro-N-(1-hydroxy-cyclohexylmethyl)-5-[methyl-(6-oxo-1,6-dihydro-pyrazin-2-yl)-amino]-benzamide;
2-Chloro-5-[(3-chloro-pyrazin-2-yl)-methyl-amino]-N-(1-hydroxy-cyclohexylmethyl)-benzamide;
2-Chloro-5-[(5-chloro-pyrazin-2-yl)-methyl-amino]-N-(1-hydroxy-cyclohexylmethyl)-benzamide;
2-Chloro-N-(1-hydroxy-cyclohexylmethyl)-5-[(5-methoxy-pyrazin-2-yl)-methyl-amino]-benzamide;
2-Chloro-5-[(6-chloro-pyridazin-3-yl)-methyl-amino]-N-(1-hydroxy-cyclohexylmethyl)-benzamide;
2-Chloro-N-(1-hydroxy-cyclohexylmethyl)-5-[methyl-(6-oxo-1,6-dihydro-pyridazin-3-yl)-amino]-benzamide;
2-Chloro-N-(1-hydroxy-cyclohexylmethyl)-5-[(6-methoxy-pyridazin-3-yl)-methyl-amino]-benzamide;
2-Chloro-N-(1-hydroxy-cyclohexylmethyl)-5-(pyridin-2-ylamino)-benzamide;
2-Chloro-N-(1-hydroxy-cyclohexylmethyl)-5-(methyl-pyridin-2-yl-amino)-benzamide;
2-Chloro-N-(1-hydroxy-cyclohexylmethyl)-5-(6-oxo-1,6-dihydro-pyridin-2-ylamino)-benzamide;
2-Chloro-N-(1-hydroxy-cyclohexylmethyl)-5-[methyl-(6-oxo-1,6-dihydro-pyridin-2-yl)-amino]-benzamide;
2-Chloro-N-(1-hydroxy-cyclohexylmethyl)-5-(1-methyl-6-oxo-1,6-dihydro-pyridin-2-ylamino)-benzamide;
2-Chloro-N-(1-hydroxy-cyclohexylmethyl)-5-[1-(2-methoxy-ethyl)-6-oxo-1,6-dihydro-pyridin-2-ylamino]-benzamide;
2-Chloro-N-(1-hydroxy-cyclohexylmethyl)-5-{[1-(2-methoxy-ethyl)-6-oxo-1,6-dihydro-pyridin-2-yl]-methyl-amino}-benzamide;
2-Chloro-N-(1-hydroxy-cyclohexylmethyl)-5-(1-methyl-2-oxo-1,2-dihydro-pyridin-3-ylamino)-benzamide;
2-Chloro-N-(1-hydroxy-cyclohexylmethyl)-5-(2-methyl-2H-pyrazol-3-ylamino)-benzamide;
2-Chloro-N-(1-hydroxy-cyclohexylmethyl)-5-[methyl-(2-methyl-2H-pyrazol-3-yl)-amino]-benzamide;
2-Chloro-5-(6-fluoro-pyridin-2-yloxy)-N-(1-hydroxy-cyclohexylmethyl)-benzamide;
2-Chloro-5-(4-fluoro-pyridin-2-yloxy)-N-(1-hydroxy-cyclohexylmethyl)-benzamide;
2-Chloro-5-(2-chloro-pyridin-4-yloxy)-N-(1-hydroxy-cyclohexylmethyl)-benzamide;
2-Chloro-N-(1-hydroxy-cyclohexylmethyl)-5-(pyridin-2-yloxy)-benzamide;
2-Chloro-N-(1-hydroxy-cyclohexylmethyl)-5-(pyridin-4-yloxy)-benzamide;
2-Chloro-N-(1-hydroxy-cyclohexylmethyl)-5-(pyridin-3-yloxy)-benzamide;
2-Chloro-5-(6-chloro-pyridin-2-yloxy)-N-(1-hydroxy-cyclohexylmethyl)-benzamide;
2-Chloro-N-(1-hydroxy-cyclohexylmethyl)-5-(6-methylamino-pyridin-2-yloxy)-benzamide;
2-Chloro-5-(6-dimethylamino-pyridin-2-yloxy)-N-(1-hydroxy-cyclohexylmethyl)-benzamide;
2-Chloro-N-(1-hydroxy-cyclohexylmethyl)-5-(6-methoxy-pyridin-2-yloxy)-benzamide;
2-Chloro-N-(1-hydroxy-cyclohexylmethyl)-5-(6-oxo-1,6-dihydro-pyridin-2-yloxy)-benzamide;
2-Chloro-N-(1-hydroxy-cyclohexylmethyl)-5-(1-methyl-6-oxo-1,6-dihydro-pyridin-2-yloxy)-benzamide;
2-Chloro-N-(1-hydroxy-cyclohexylmethyl)-5-(pyrimidin-4-yloxy)-benzamide;
2-Chloro-5-(2-chloro-pyrimidin-4-yloxy)-N-(1-hydroxy-cyclohexylmethyl)-benzamide;
2-Chloro-N-(1-hydroxy-cyclohexylmethyl)-5-(2-methylamino-pyrimidin-4-yloxy)-benzamide;
2-Chloro-5-(2-dimethylamino-pyrimidin-4-yloxy)-N-(1-hydroxy-cyclohexylmethyl)-benzamide;
2-Chloro-N-(4,4-difluoro-1-hydroxy-cyclohexylmethyl)-5-(2-oxo-1,2-dihydro-pyrimidin-4-yloxy)-benzamide;
2-Chloro-N-(1-hydroxy-cyclohexylmethyl)-5-(2-oxo-2,3-dihydro-pyrimidin-4-yloxy)-benzamide;
2-Chloro-N-(1-hydroxy-cyclohexylmethyl)-5-(2-methoxy-pyrimidin-4-yloxy)-benzamide;
2-Chloro-N-(1-hydroxy-cyclohexylmethyl)-5-(pyrazin-2-yloxy)-benzamide;
2-Chloro-5-(6-chloro-pyrazin-2-yloxy)-N-(1-hydroxy-cyclohexylmethyl)-benzamide;
2-Chloro-N-(1-hydroxy-cyclohexylmethyl)-5-(6-methoxy-pyrazin-2-yloxy)-benzamide;
2-Chloro-N-(1-hydroxy-cyclohexylmethyl)-5-(6-methylamino-pyrazin-2-yloxy)-benzamide;
2-Chloro-5-(6-dimethylamino-pyrazin-2-yloxy)-N-(1-hydroxy-cyclohexylmethyl)-benzamide;
2-Chloro-N-(1-hydroxy-cyclohexylmethyl)-5-(6-oxo-1,6-dihydro-pyrazin-2-yloxy)-benzamide;
2-Chloro-5-(6-chloro-pyridazin-3-yloxy)-N-(1-hydroxy-cyclohexylmethyl)-benzamide;
2-Chloro-N-(1-hydroxy-cyclohexylmethyl)-5-(6-methoxy-pyridazin-3-yloxy)-benzamide;
2-Chloro-N-(1-hydroxy-cyclohexylmethyl)-5-(6-oxo-1,6-dihydro-pyridazin-3-yloxy)-benzamide;
2-Chloro-N-(1-hydroxy-cyclohexylmethyl)-5-(pyridazin-3-yloxy)-benzamide;
2-Chloro-N-(1-hydroxy-cyclohexylmethyl)-5-(5-methoxy-pyridazin-3-yloxy)-benzamide;
2-Chloro-N-(1-hydroxy-cyclohexylmethyl)-5-(pyridin-2-ylsulfanyl)-benzamide;
2-Chloro-N-(1-hydroxy-cyclohexylmethyl)-5-(pyridine-2-sulfinyl)-benzamide;
2-Chloro-N-(1-hydroxy-cyclohexylmethyl)-5-(pyridine-2-sulfonyl)-benzamide;
2-Chloro-5-(2-chloro-pyrimidin-4-ylsulfanyl)-N-(1-hydroxy-cyclohexylmethyl)-benzamide;
2-Chloro-5-(2-chloro-pyrimidine-4-sulfinyl)-N-(1-hydroxy-cyclohexylmethyl)-benzamide;
2-Chloro-N-(1-hydroxy-cyclohexylmethyl)-5-(2-oxo-1,2-dihydro-pyrimidin-4-ylsulfanyl)-benzamide;
2-Chloro-N-(4,4-difluoro-1-hydroxy-cyclohexylmethyl)-5-(2-oxo-1,2-dihydro-pyrimidin-4-ylsulfanyl)-benzamide;
2-Chloro-N-(1-hydroxy-cyclohexylmethyl)-5-(2-methoxy-pyrimidin-4-ylsulfanyl)-benzamide;
2-Chloro-N-(1-hydroxy-cyclohexylmethyl)-5-(2-methoxy-pyrimidine-4-sulfinyl)-benzamide;
2-Chloro-N-(1-hydroxy-cyclohexylmethyl)-5-(2-methoxy-pyrimidine-4-sulfonyl)-benzamide;
2-Chloro-N-(1-hydroxy-cyclohexylmethyl)-5-(2-oxo-2H-pyridin-1-ylmethyl)-benzamide;
2-Chloro-N-(1-hydroxy-cyclohexylmethyl)-5-(2-oxo-2H-pyrimidin-1-ylmethyl)-benzamide;
2-Chloro-5-(2,4-dioxo-3,4-dihydro-2H-pyrimidin-1-ylmethyl)-N-(1-hydroxy-cyclohexylmethyl)-benzamide;
2-Chloro-N-(1-hydroxy-cyclohexylmethyl)-5-pyridin-2-ylmethyl-benzamide;
2-Chloro-N-(1-hydroxy-cyclohexylmethyl)-5-(2-methylsulfanyl-pyrimidin-4-ylmethyl)-benzamide;
2-Chloro-N-(1-hydroxy-cyclohexylmethyl)-5-(2-oxo-1,2-dihydro-pyrimidin-4-ylmethyl)-benzamide;
2-Chloro-N-(1-hydroxy-cyclohexylmethyl)-5-(2-methoxy-pyrimidin-4-ylmethyl)-benzamide;
2-Chloro-N-(4,4-difluoro-1-hydroxy-cyclohexylmethyl)-5-(2-oxo-1,2-dihydro-pyrimidin-4-ylmethyl)-benzamide;
2-Chloro-N-(1-hydroxy-cyclohexylmethyl)-5-(2-oxo-1,2-dihydro-pyrimidine-4-carbonyl)-benzamide;
2-Chloro-5-[difluoro-(2-oxo-1,2-dihydro-pyrimidin-4-yl)-methyl]-N-(1-hydroxy-cyclohexylmethyl)-benzamide;
2-Chloro-5-[fluoro-(2-oxo-1,2-dihydro-pyrimidin-4-yl)-methyl]-N-(1-hydroxy-cyclohexylmethyl)-benzamide;
5-[(2-Chloro-pyrimidin-4-yl)-methyl-amino]-N-(1-hydroxy-cyclohexylmethyl)-2-methyl-benzamide;
N-(1-Hydroxy-cyclohexylmethyl)-5-[(2-methoxy-pyrimidin-4-yl)-methyl-amino]-2-methyl-benzamide;
N-(1-Hydroxy-cyclohexylmethyl)-2-methyl-5-[methyl-(2-oxo-1,2-dihydro-pyrimidin-4-yl)-amino]-benzamide;
2-Chloro-N-(1-hydroxy-cyclohexylmethyl)-5-[1-hydroxy-1-(2-oxo-1,2-dihydro-pyrimidin-4-yl)-ethyl]-benzamide;
2-Chloro-N-(1-hydroxy-cyclohexylmethyl)-5-[(1S)-1-hydroxy-1-(2-oxo-1,2-dihydro-pyrimidin-4-yl)-ethyl]-benzamide;
2-Chloro-N-(1-hydroxy-cyclohexylmethyl)-5-[(1R)-1-hydroxy-1-(2-oxo-1,2-dihydro-pyrimidin-4-yl)-ethyl]-benzamide; and
2-Chloro-N-(1-hydroxy-cyclohexylmethyl)-5-[1-hydroxy-1-(2-methoxy-pyrimidin-4-yl)-ethyl]-benzamide;
or a salt of such a compound.

12. (canceled)

13. A pharmaceutical composition containing, as active principle, a compound of formula (I) according to claim 1 or a pharmaceutically acceptable salt thereof, and at least one therapeutically inert excipient.

14. A method for the prevention or treatment of a disease selected from pain; neurodegenerative and neuroinflammatory diseases; bone and joint diseases; obstructive diseases of the airways; cardiovascular diseases; eye diseases; skin diseases; abdominal and gastrointestinal tract diseases; genitourinary diseases; cancer; other auto-immune and allergic disorders; and other disorders with an inflammatory or immunological component comprising administering to a patient in need thereof an effective amount of a compound of formula (I) according to claim 1, or of a pharmaceutically acceptable salt thereof.

15. (canceled)


SEQUENCE LISTING (2 sequences)

SEQ ID NO: 1 - DNA, 35 bp, Homo sapiens
  source 1..35: /mol_type=“DNA” /organism=“Homo sapiens”
atcgcggccgctcagtaaggactcttgaagccact

SEQ ID NO: 2 - DNA, 38 bp, Homo sapiens
  source 1..38: /mol_type=“DNA” /organism=“Homo sapiens”
cgccgctagcaccaccatgccggcctgctgcagctgca